(19) United States
(12) Patent Application Publication
(10) Pub. No.: US 2024/0342308 A1
(43) Pub. Date: Oct. 17, 2024

(54) Methods and Compositions for Treating Conditions Involving the Eye

(71) Applicant: Krystal Biotech, Inc., Pittsburgh, PA (US)

(72) Inventors: Suma Krishnan, Pittsburgh, PA (US); Haley Nicole Cartwright, Pittsburgh, PA (US)

(21) Appl. No.: 18/634,382

(22) Filed: Apr. 12, 2024

Related U.S. Application Data
(60) Provisional application No. 63/459,102, filed on Apr. 13, 2023.
(60) Provisional application No. 63/460,948, filed on Apr. 21, 2023.
(60) Provisional application No. 63/593,085, filed on Oct. 25, 2023.

(51) Int. Cl. A61K 48/00; A61K 38/18; A61P 27/02; C12N 15/86

(52) U.S. Cl. CPC A61K 48/0033; A61K 38/185; A61K 48/0075; A61P 27/02; C12N 15/86; C12N 2710/16622; C12N 2710/16643; C12N 2710/16652; C12N 2710/16662

(57) ABSTRACT

The present disclosure provides recombinant nucleic acids comprising one or more polynucleotides encoding a polypeptide; viruses comprising the recombinant nucleic acids; compositions and formulations comprising the recombinant nucleic acids and/or viruses; methods of their use (e.g., for the treatment of one or more conditions or diseases of the eye); and articles of manufacture or kits thereof.

### CROSS REFERENCE TO RELATED APPLICATIONS

This application claims the priority benefit of U.S. Provisional Application Ser. No. 63/459,102, filed Apr. 13, 2023, U.S. Provisional Application Ser. No. 63/460,948, filed Apr. 21, 2023, and U.S. Provisional Application Ser. No. 63/593,085, filed Oct. 25, 2023, the contents of which are incorporated herein by reference in their entirety.

### SEQUENCE LISTING INFORMATION

The Sequence Listing associated with this application is filed in electronic format via Patent Center and is hereby incorporated by reference into the specification in its entirety. The name of the file containing the Sequence Listing is 2401989.xml. The size of the file is 1,738,822 bytes, and the file was created on Apr. 12, 2024.

### FIELD OF THE INVENTION

The present disclosure relates, in part, to recombinant nucleic acids comprising one or more polynucleotides encoding a polypeptide, viruses comprising the same, pharmaceutical compositions and formulations thereof, and methods of their use (e.g., for treating one or more conditions or diseases of the eye).

### BACKGROUND

Conditions of the eye, vision loss, eye disease, and/or vision disorders carry a significant economic burden with an annual estimated cost of about $139 billion in the U.S. Despite significant advances in clinical care and treatment methods, more effective therapeutic options are still needed.

All references cited herein, including patent applications, patent publications, non-patent literature, and NCBI/UniProtKB/Swiss-Prot Accession numbers are herein incorporated by reference in their entirety, as if each individual reference were specifically and individually indicated to be incorporated by reference.

### BRIEF SUMMARY

In order to meet these and other needs, provided herein are recombinant nucleic acids (e.g., recombinant herpes virus genomes) encoding one or more polypeptides, viruses (e.g., herpes viruses) comprising the recombinant nucleic acids (e.g., recombinant herpes virus genomes), pharmaceutical compositions and formulations, medicaments, and/or methods of administration (e.g., useful for treating an eye condition or disease) in a subject in need thereof.

Accordingly, certain aspects of the present disclosure relate to a recombinant herpes virus genome comprising one or more polynucleotides encoding a polypeptide. In some embodiments, the recombinant herpes virus genome comprises two or more polynucleotides encoding a polypeptide. In some embodiments, the recombinant herpes virus genome is replication competent. In some embodiments, the recombinant herpes virus genome is replication defective. In some embodiments that may be combined with any of the preceding embodiments, the recombinant herpes virus genome comprises the one or more polynucleotides encoding the polypeptide within one or more viral gene loci. In some embodiments that may be combined with any of the preceding embodiments, the recombinant herpes virus genome is selected from a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, an Epstein-Barr virus genome, a recombinant Kaposi's sarcoma-associated herpesvirus genome, and any combinations or derivatives thereof.

In some embodiments that may be combined with any of the preceding embodiments, the recombinant herpes virus genome is a recombinant herpes simplex virus genome. In some embodiments, the recombinant herpes simplex virus genome is a recombinant type 1 herpes simplex virus (HSV-1) genome, a recombinant type 2 herpes simplex virus (HSV-2) genome, or any combinations or derivatives thereof. In some embodiments, the recombinant herpes simplex virus genome is a recombinant HSV-1 genome.

In some embodiments that may be combined with any of the preceding embodiments, the recombinant herpes simplex virus genome has been engineered to reduce or eliminate expression of one or more herpes simplex virus genes (e.g., one or more toxic herpes simplex virus genes). In some embodiments that may be combined with any of the preceding embodiments, the recombinant herpes simplex virus genome comprises an inactivating mutation. In some embodiments, the inactivating mutation is in a herpes simplex virus gene. In some embodiments, the inactivating mutation is a deletion of at least a portion of the coding sequence of the herpes simplex virus gene. In some embodiments, the inactivating mutation is a deletion of the coding sequence of the herpes simplex virus gene. In some embodiments, the herpes simplex virus gene is selected from Infected Cell Protein (ICP) 0 (one or both copies), ICP4 (one or both copies), ICP22, ICP27, ICP47, thymidine kinase (tk), Long Unique Region (UL) 41, and UL55. In some embodiments that may be combined with any of the preceding embodiments, the recombinant herpes simplex virus genome comprises an inactivating mutation in one or both copies of the ICP4 gene. In some embodiments that may be combined with any of the preceding embodiments, the recombinant herpes simplex virus genome comprises an inactivating mutation in the ICP22 gene. In some embodiments that may be combined with any of the preceding embodiments, the recombinant herpes simplex virus genome comprises an inactivating mutation in the UL41 gene. In some embodiments that may be combined with any of the preceding embodiments, the recombinant herpes simplex virus genome comprises an inactivating mutation in one or both copies of the ICP0 gene. In some embodiments that may be combined with any of the preceding embodiments, the recombinant herpes simplex virus genome comprises an inactivating mutation in the ICP27 gene. In some embodiments that may be combined with any of the preceding embodiments, the recombinant herpes simplex virus genome comprises an inactivating mutation in the UL55 gene. In some embodiments that may be combined with any of the preceding embodiments, the recombinant herpes simplex virus genome does not comprise an inactivating mutation in the ICP47 gene. In some embodiments, the recombinant herpes simplex virus genome does not comprise an inactivating mutation in one or both copies of the ICP34.5 gene. In some embodiments that may be combined with any of the preceding embodiments, the recombinant herpes simplex virus genome does not comprise an inactivating mutation in the UL36 gene.

In some embodiments that may be combined with any of the preceding embodiments, the recombinant herpes simplex virus genome comprises the one or more polynucleotides encoding the polypeptide within one or more viral gene loci. In some embodiments that may be combined with any of the preceding embodiments, the recombinant herpes simplex virus genome comprises the one or more polynucleotides encoding the polypeptide within one or both of the ICP4 viral gene loci. In some embodiments that may be combined with any of the preceding embodiments, the recombinant herpes simplex virus genome comprises the one or more polynucleotides encoding the polypeptide within the ICP22 viral gene locus. In some embodiments that may be combined with any of the preceding embodiments, the recombinant herpes simplex virus genome comprises the one or more polynucleotides encoding the polypeptide within the UL41 viral gene locus. In some embodiments that may be combined with any of the preceding embodiments, the recombinant herpes simplex virus genome comprises the one or more polynucleotides encoding the polypeptide within one or both of the ICP0 viral gene loci. In some embodiments that may be combined with any of the preceding embodiments, the recombinant herpes simplex virus genome comprises the one or more polynucleotides encoding the polypeptide within the ICP27 viral gene locus. In some embodiments that may be combined with any of the preceding embodiments, the recombinant herpes simplex virus genome comprises the one or more polynucleotides encoding the polypeptide within the UL55 viral gene locus.

In some embodiments that may be combined with any of the preceding embodiments, the polypeptide is a human polypeptide. In some embodiments that may be combined with any of the preceding embodiments, the polypeptide is selected from the group consisting of sterile alpha motif domain-containing protein 11, nephrocystin-4, espin, nicotinamide/nicotinic acid mononucleotide adenylyltransferase 1, mitofusin-2, ER membrane protein complex subunit 1, phospholipase A2 group V, dehydrodolichyl diphosphate synthase complex subunit, palmitoyl-protein thioesterase 1, elongation of very long chain fatty acids protein 1, Protein O-linked-mannose beta-1,2-N-acetylglucosaminyltransferase 1, Retinoid isomerohydrolase, Retinal-specific phospholipid-transporting ATPase, Collagen alpha-1 (XI), Guanine nucleotide-binding protein G(t) subunit alpha-2, Chloride channel CLIC-like protein 1, DNA damage-regulated autophagy modulator protein 2, U4/U6 small nuclear ribonucleoprotein Prp3, Alpha-endosulfine, Semaphorin-4A, Cyclic AMP-dependent transcription factor ATF-6 alpha, Hemicentin-1, Complement factor H, Protein crumbs homolog 1, Adiponectin receptor protein 1, Protein RD3, Serine/threonine-protein kinase Nek2, Feline leukemia virus subgroup C receptor-related protein 1, Usherin, Serologically defined colon cancer antigen 8, Olfactory receptor 2W3, NBAS subunit of NRZ tethering complex, Cytosolic carboxypeptidase-like protein 5, Zinc finger protein 513, Intraflagellar transport protein 172 homolog, Photoreceptor cilium actin regulator, EGF-containing fibulin-like extracellular matrix protein 1/TLE family member 5, Protein FAM161A, WD repeat-containing and planar cell polarity effector protein fritz homolog, Centrosome-associated protein ALMS1, U5 small nuclear ribonucleoprotein 200 kDa helicase, Metal transporter CNNM4, Cyclic nucleotide-gated cation channel alpha-3, Nephrocystin-1, Tyrosine-protein kinase Mer, Bardet-Biedl syndrome 5 protein, Ceramide kinase-like protein, Neurogenic differentiation factor 1, Transmembrane protein 237, Inward rectifier potassium channel 13, S-arrestin, Secreted phosphoprotein 24, CCA tRNA nucleotidyltransferase 1, mitochondrial, Sodium bicarbonate cotransporter 3, Leucine zipper transcription factor-like protein 1, Guanine nucleotide-binding protein G(t) subunit alpha-1, Three-prime repair exonuclease 1, MAP kinase-activated protein kinase 3, Ataxin-7, Vitamin K-dependent protein S, ADP-ribosylation factor-like protein 6, Interphotoreceptor matrix proteoglycan 2, IQ calmodulin-binding motif-containing protein 1, Rhodopsin, Nephrocystin-3, Clarin-1, Probable cationic amino acid transporter/Solute carrier family 7 member 14, Choline-phosphate cytidylyltransferase A, Centrosomal protein of 19 kDa, Rod cGMP-specific 3′,5′-cyclic phosphodiesterase subunit beta, Wolframin, Homeobox protein HMX1, Ras-related protein Rab-28, Coiled-coil and C2 domain-containing protein 2A, Prominin-1, Adhesion G protein-coupled receptor A3, Death domain-containing protein 1, WD repeat-containing protein 19, cGMP-gated cation channel alpha-1, CDGSH iron-sulfur domain-containing protein 2, Microsomal triglyceride transfer protein large subunit, Leucine-rich repeat, immunoglobulin-like domain and transmembrane domain-containing protein 3, Bardet-Biedl syndrome 7 protein, Bardet-Biedl syndrome 12 protein, Major facilitator superfamily domain-containing protein 8, Serine/threonine-protein kinase PLK4, Lecithin retinol acyltransferase, Toll-like receptor 3, Cytochrome P450 4V2, Spliceosome-associated protein CWC27 homolog, Centrosomal protein POC5, Versican core protein, Adhesion G-protein coupled receptor V1, COUP transcription factor 1, Mitochondrial outer membrane protein SLC25A46, Catenin alpha-1, Histidine-tRNA ligase, cytoplasmic, Rod cGMP-specific 3′,5′-cyclic phosphodiesterase subunit alpha, Metabotropic glutamate receptor 6, Serine/threonine-protein kinase MAK, Complement C2, Complement factor B, Tubby-related protein 1, Guanylyl cyclase-activating protein 1, Guanylyl cyclase-activating protein 2, Peripherin-2, Interphotoreceptor matrix proteoglycan 1, Protein eyes shut homolog, Collagen alpha-1 (IX) chain, Regulating synaptic membrane exocytosis protein 1, Lebercilin, Elongation of very long chain fatty acids protein 4, PR domain zinc finger protein 13, Reticulon-4-interacting protein 1, mitochondrial, Jouberin, Peroxisomal targeting signal 2 receptor, CCR4-NOT transcription complex subunit 9, Aryl hydrocarbon receptor, Kelch-like protein 7, Retinitis pigmentosa 9 protein, Protein PTHB1, Peroxisomal ATPase PEX1, Tetraspanin-12, Inosine-5′-monophosphate dehydrogenase 1, Short-wave-sensitive opsin 1, UPF0606 protein KIAA1549, Retinitis pigmentosa 1-like 1 protein, Disintegrin and metalloproteinase domain-containing protein 9, Heparan-alpha-glucosaminide N-acetyltransferase, Oxygen-regulated protein 1, Alpha-tocopherol transfer protein, Centrosome and spindle pole-associated protein 1, Dynamin-like 120 kDa protein, mitochondrial, Peroxisome biogenesis factor 2, Cyclic nucleotide-gated cation channel beta-3, Cilia- and flagella-associated protein 418, Growth/differentiation factor 6, Regulating synaptic membrane exocytosis protein 2, Potassium voltage-gated channel subfamily V member 2, E3 ubiquitin-protein ligase Topors, Centrosomal protein of 78 kDa, Inversin, U4/U6 small nuclear ribonucleoprotein Prp4, Whirlin, E3 ubiquitin-protein ligase TRIM32, Toll-like receptor 4, Cytoplasmic dynein 2 intermediate chain 2, Programmed cell death protein 2, Exosome complex component RRP4, Phosphatidylinositol polyphosphate 5-phosphatase type IV, Phytanoyl-CoA dioxygenase, peroxisomal, Acyl-CoA-binding domain-containing protein, Protocadherin-15, Retinol-binding protein 3, DNA excision repair protein ERCC-6, Hexokinase-1, Cadherin-23, Cadherin-related family member 1, RPE-retinal G protein-coupled receptor, Kinesin-like protein KIF11, Retinol-binding protein 4, Cone cGMP-specific 3′,5′-cyclic phosphodiesterase subunit alpha, Paired box protein Pax-2, PDZ domain-containing protein 7, ADP-ribosylation factor-like protein 3, BBSome-interacting protein 1, Age-related maculopathy susceptibility protein 2, Serine protease HTRA1, Ornithine aminotransferase, mitochondrial, Zinc finger protein 408, Tubby protein homolog, Transcriptional enhancer factor TEF-1, Harmonin, Transmembrane protein 216, Bestrophin-1, Isoaspartyl peptidase/L-asparaginase, Rod outer segment membrane protein 1, Bardet-Biedl syndrome 1 protein, Calcium-binding protein 4, Low-density lipoprotein receptor-related protein 5, Calpain-5, Unconventional myosin-VIIa, Transmembrane protein 126A, Frizzled-4, Cytoplasmic dynein 2 light intermediate chain 1, Centrosomal protein of 164 kDa, Complement C1q tumor necrosis factor-related protein 5, Membrane frizzled-related protein, Voltage-dependent calcium channel subunit alpha-2/delta-4, Guanine nucleotide-binding protein G(I)/G(S)/G(T) subunit beta-3, Retinal cone rhodopsin-sensitive cGMP 3′,5′-cyclic phosphodiesterase subunit gamma, Collagen alpha-1 (II) chain, Matrix metalloproteinase-19, Retinol dehydrogenase 5, T-complex protein 1 subunit beta, Bardet-Biedl syndrome 10 protein, Centrosomal protein of 290 kDa, POC1 centriolar protein homolog B, Mevalonate kinase, Intraflagellar transport protein 81 homolog, Mitochondrial translation release factor in rescue, Integral membrane protein 2B, Retinoblastoma-associated protein, RCC1 and BTB domain-containing protein 1, Rhodopsin kinase GRK1, X-linked retinitis pigmentosa GTPase regulator-interacting protein 1, Neural retina-specific leucine zipper protein, Homeobox protein OTX2, Retinol dehydrogenase 11, Retinol dehydrogenase 12, Tubulin polyglutamylase TTLL5, Spermatogenesis-associated protein 7, Tetratricopeptide repeat protein 8, Fibulin-5, Transient receptor potential cation channel subfamily M member 1, Gamma-tubulin complex component 4, Sodium/potassium/calcium exchanger 1, Photoreceptor-specific nuclear receptor, Bardet-Biedl syndrome 4 protein, Calcium and integrin-binding family member 2, Retinaldehyde-binding protein 1, N-acetylglucosamine-1-phosphotransferase subunit gamma, Intraflagellar transport protein 140 homolog, Clusterin-associated protein 1, ATP-binding cassette sub-family C member 6, Ketimine reductase mu-crystallin, Battenin, Zinc finger protein 423, Protein fantom, Bardet-Biedl syndrome 2 protein, ADP-ribosylation factor-like protein 2-binding protein, Cyclic nucleotide-gated cation channel beta-1, Cadherin-3, Pre-mRNA-splicing factor ATP-dependent RNA helicase PRP16, A disintegrin and metalloproteinase with thrombospondin motifs 18, Solute carrier family 38 member 8, Retinal guanylyl cyclase 1, Pre-mRNA-processing-splicing factor 8, Aryl-hydrocarbon-interacting protein-like 1, Membrane-associated phosphatidylinositol transfer protein 3, Protein unc-119 homolog A, Probable G-protein coupled receptor 179, Tectonic-like complex member MKS1, Carbonic anhydrase 4, Regulator of G-protein signaling 9, Arylsulfatase G, pre-mRNA splicing regulator USH1G, Photoreceptor disk component PRCD, Fascin-2, Retinal rod rhodopsin-sensitive cGMP 3′,5′-cyclic phosphodiesterase subunit gamma, Laminin subunit alpha-1, AFG3-like protein 2, Cone-rod homeobox protein, Receptor expression-enhancing protein 6, Retina and anterior neural fold homeobox protein 2, Complement C3, Rho guanine nucleotide exchange factor 18, Patatin-like phospholipase domain-containing protein 6, Regulator of G-protein signaling 9-binding protein, Optic atrophy 3 protein, U4/U6 small nuclear ribonucleoprotein Prp31, Isocitrate dehydrogenase [NAD] subunit beta, mitochondrial, Pantothenate kinase 2, mitochondrial, Protein jagged-1, Molecular chaperone MKKS, Centrosomal protein kizuna, Lysophosphatidylserine lipase ABHD12, Kinesin-like protein KIF3B, Centrosome-associated protein CEP250, Pre-mRNA-processing factor 6, Cilia- and flagella-associated protein 410, Dynamin-1-like protein, Metalloproteinase inhibitor 3, Intraflagellar transport protein 27 homolog, Fibulin-1, MIEF1 upstream open reading frame protein, Aconitate hydratase, mitochondrial, Gamma-tubulin complex component 6, Centriole and centriolar satellite protein, Retinoschisin, Protein XRP2, Dystrophin, X-linked retinitis pigmentosa GTPase regulator, Nyctalopin, Xaa-Pro dipeptidase, Norrin, Voltage-dependent L-type calcium channel subunit alpha-1F, Phosphoglycerate kinase 1, Rab proteins geranylgeranyltransferase component A 1, Mitochondrial import inner membrane translocase subunit Tim8 A, Ribose-phosphate pyrophosphokinase 1, Long-wave-sensitive opsin 1, Medium-wave-sensitive opsin 1, Short-wave-sensitive opsin 1, Transcription factor A, mitochondrial, NADH-ubiquinone oxidoreductase chain 1, NADH-ubiquinone oxidoreductase chain 2, NADH-ubiquinone oxidoreductase chain 3, NADH-ubiquinone oxidoreductase chain 4L, NADH-ubiquinone oxidoreductase chain 4, NADH-ubiquinone oxidoreductase chain 5, NADH-ubiquinone oxidoreductase chain 6, ATP synthase subunit a, ATP synthase protein 8, Cytochrome c oxidase subunit 1, Cytochrome c oxidase subunit 3, Cytochrome b, Leucine-tRNA ligase, mitochondrial, Nondiscriminating glutamyl-tRNA synthetase EARS2, mitochondrial, Lysine-tRNA ligase, Histidine-tRNA ligase, mitochondrial, Serine-tRNA ligase, mitochondrial, Probable proline-tRNA ligase, mitochondrial, Cyanocobalamin reductase/alkylcobalamin dealkylase, POU domain, class 3, transcription factor 4, Ribosomal protein S6 kinase alpha-6, Ciliogenesis and planar polarity effector 1, Meckelin, TRAF3-interacting protein 1, Intraflagellar transport protein 74 homolog, S phase cyclin A-associated protein in the endoplasmic reticulum, Sodium channel and clathrin linker 1, Protein TALPID3, Tectonic-2, ADP-ribosylation factor-like protein 13B, B9 domain-containing protein 1, B9 domain-containing protein 2, C2 domain-containing protein 3, Centrosomal protein of 41 kDa, Centrosomal protein of 104 kDa, Centrosomal protein of 120 kDa, Intraflagellar transport protein 172 homolog, Katanin-interacting protein, Kinesin-like protein KIF7, Retinal rod rhodopsin-sensitive cGMP 3′,5′-cyclic phosphodiesterase subunit delta, Tectonic-1, Tectonic-3, Transmembrane protein 107, Transmembrane protein 138, Transmembrane protein 231, Tetratricopeptide repeat protein 21B, Nuclear receptor ROR-alpha, Beta-nerve growth factor, Collagen alpha-2 (VIII) chain, Solute carrier family 4 member 11, Zinc finger E-box-binding homeobox 1, Keratin, type II cuticular Hb3, Keratin, type I cytoskeletal 12, Transforming growth factor-beta-induced protein ig-h3, Tumor-associated calcium signal transducer 2, Carbohydrate sulfotransferase 6, Gelsolin, UbiA prenyltransferase domain-containing protein 1, Decorin, 1-phosphatidylinositol 3-phosphate 5-kinase, Transcription factor Ovo-like 2, and Grainyhead-like protein 2 homolog.

In some embodiments that may be combined with any of the preceding embodiments, the recombinant herpes virus genome has reduced cytotoxicity when introduced into a target cell as compared to a corresponding wild-type herpes virus genome. In some embodiments, the target cell is a human cell. In some embodiments, the target cell is a cell of the eye. In some embodiments, the target cell is a corneal epithelial cell. In some embodiments, the target cell is a cell of the retina. In some embodiments, the target cell is a retinal pigment epithelial cell. In some embodiments, the target cell is a photoreceptor.

Other aspects of the present disclosure relate to a herpes virus comprising any of the recombinant herpes virus genomes described herein. In some embodiments, the herpes virus is replication competent. In some embodiments, the herpes virus is replication defective. In some embodiments that may be combined with any of the preceding embodiments, the herpes virus is not oncolytic. In some embodiments that may be combined with any of the preceding embodiments, the herpes virus is a pseudotyped virus. In some embodiments that may be combined with any of the preceding embodiments, the herpes virus is not a pseudotyped virus. In some embodiments that may be combined with any of the preceding embodiments, the herpes virus is not a pseudotyped oncolytic virus. In some embodiments that may be combined with any of the preceding embodiments, the herpes virus has reduced cytotoxicity as compared to a corresponding wild-type herpes virus. In some embodiments that may be combined with any of the preceding embodiments, the herpes virus is selected from a herpes simplex virus, a varicella zoster virus, a human cytomegalovirus, a herpesvirus 6A, a herpesvirus 6B, a herpesvirus 7, an Epstein-Barr virus, a Kaposi's sarcoma-associated herpesvirus, and any combinations or derivatives thereof. In some embodiments that may be combined with any of the preceding embodiments, the herpes virus is a herpes simplex virus. In some embodiments, the herpes simplex virus is not oncolytic. In some embodiments, the herpes simplex virus is an HSV-1, an HSV-2, or any combinations or derivatives thereof. In some embodiments, the herpes simplex virus is an HSV-1. In some embodiments, the HSV-1 is not oncolytic.

Other aspects of the present disclosure relate to a pharmaceutical composition comprising any of the recombinant herpes virus genomes and/or any of the recombinant herpes viruses described herein and a pharmaceutically acceptable carrier or excipient. In some embodiments that may be combined with any of the preceding embodiments, the pharmaceutical composition is suitable for ocular, subretinal, intraocular, intravitreal, topical, subcutaneous, subconjunctival, subtenon, subtenon capsule, intracameral, retrobulbar, systemic, parenteral, periocular, juxtascleral, anterior juxtascleral, posterior juxtascleral, oral, peribulbar, or suprachoroidal administration. In some embodiments that may be combined with any of the preceding embodiments, the pharmaceutical composition comprises a phosphate buffer. In some embodiments that may be combined with any of the preceding embodiments, the pharmaceutical composition comprises glycerol. In some embodiments that may be combined with any of the preceding embodiments, the pharmaceutical composition comprises a lipid carrier. In some embodiments that may be combined with any of the preceding embodiments, the pharmaceutical composition comprises a nanoparticle carrier.

Other aspects of the present disclosure relate to the use of any of the recombinant nucleic acids (e.g., recombinant herpes virus genomes), recombinant viruses (e.g., recombinant herpes viruses), and/or pharmaceutical compositions described herein as a medicament.

Other aspects of the present disclosure relate to the use of any of the recombinant nucleic acids (e.g., recombinant herpes virus genomes), recombinant viruses (e.g., recombinant herpes viruses), and/or pharmaceutical compositions described herein in a therapy.

Other aspects of the present disclosure relate to the use of any of the recombinant nucleic acids (e.g., recombinant herpes virus genomes), recombinant viruses (e.g., recombinant herpes viruses), and/or pharmaceutical compositions described herein in the preparation of a medicament for treating an eye condition or disease.

Other aspects of the present disclosure relate to a method of expressing, enhancing, increasing, augmenting, and/or supplementing the levels of a polypeptide in one or more cells of a subject comprising administering to the subject an effective amount of any of the recombinant herpes viruses and/or pharmaceutical compositions described herein. In some embodiments, the one or more cells are one or more cells of the eye. In some embodiments that may be combined with any of the preceding embodiments, the subject is a human. In some embodiments that may be combined with any of the preceding embodiments, the herpes virus or pharmaceutical composition is administered via injection, via injection to the eye, via subretinal injection, via intraocular injection, via intravitreal injection, via suprachoroidal injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally.

Other aspects of the present disclosure relate to a method of providing prophylactic, palliative, or therapeutic relief of one or more signs or symptoms of an eye condition or disease in a subject in need thereof comprising administering to the subject an effective amount of any of the recombinant herpes viruses and/or pharmaceutical compositions described herein. In some embodiments, the eye condition or disease is selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital night blindness, congenital stationary night blindness, severe congenital stationary night blindness, recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and calcific band keratopathy. In some embodiments, the eye condition or disease is Stargardt syndrome. In some embodiments, the eye condition or disease is neurotrophic keratitis. In some embodiments, the eye condition or disease is Leber congenital amaurosis. In some embodiments that may be combined with any of the preceding embodiments, the subject is a human. In some embodiments that may be combined with any of the preceding embodiments, the herpes virus or pharmaceutical composition is administered via injection, via injection to the eye, via subretinal injection, via intraocular injection, via intravitreal injection, via suprachoroidal injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally to the subject.

Other aspects of the present disclosure relate to any of the recombinant herpes viruses and/or pharmaceutical compositions described herein for use in the treatment of an eye condition or disease in a subject in need thereof.

Other aspects of the present disclosure relate to a method of treating an eye condition or disease in a subject in need thereof comprising administering to the subject an effective amount of any of the recombinant herpes viruses and/or pharmaceutical compositions described herein.

In any of the preceding embodiments, the eye condition or disease can be selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital night blindness, congenital stationary night blindness, severe congenital stationary night blindness, recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with dystrophy, chorioretinal hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and calcific band keratopathy. In some embodiments, the eye condition or disease is Stargardt syndrome. In some embodiments, the eye condition or disease is neurotrophic keratitis. In some embodiments, the eye condition or disease is Leber congenital amaurosis. In some embodiments that may be combined with any of the preceding embodiments, the subject is a human. In some embodiments that may be combined with any of the preceding embodiments, the herpes virus or pharmaceutical composition is administered via injection, via injection to the eye, via subretinal injection, via intraocular injection, via intravitreal injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally to the subject.

Other aspects of the present disclosure relate to a method of delivering a polypeptide to one or more cells of the eye a subject comprising administering to the subject a pharmaceutical composition comprising (a) a herpes virus comprising a recombinant herpes virus genome, wherein the recombinant herpes virus genome comprises one or more polynucleotides encoding the polypeptide, and (b) a pharmaceutically acceptable carrier. In some embodiments, the subject suffers from a disease or condition affecting one or more cells of the eye.

In some embodiments that may be combined with any of the preceding embodiments, the herpes virus is a pseudotyped virus. In some embodiments that may be combined with any of the preceding embodiments, the herpes virus is not a pseudotyped virus. In some embodiments that may be combined with any of the preceding embodiments, the herpes virus is not a pseudotyped oncolytic virus.

Other aspects of the present disclosure relate to an article of manufacture or kit comprising any of the recombinant nucleic acids, viruses, medicaments, and/or pharmaceutical compositions or formulations described herein and instructions for administration thereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-11 show schematics of wild-type and modified herpes simplex virus genomes. FIG. 1A shows a wild-type herpes simplex virus genome. FIG. 1B shows a modified herpes simplex virus genome comprising deletions of the coding sequence of ICP4 (both copies), with an expression cassette containing a nucleic acid encoding a polypeptide integrated at each of the ICP4 loci. FIG. 1C shows a modified herpes simplex virus genome comprising deletions of the coding sequences of ICP4 (both copies) and UL41, with an expression cassette containing a nucleic acid encoding a polypeptide integrated at each of the ICP4 loci. FIG. 1D shows a modified herpes simplex virus genome comprising deletions of the coding sequences of ICP4 (both copies) and UL41, with an expression cassette containing a nucleic acid encoding a polypeptide integrated at the UL41 locus. FIG. 1E shows a modified herpes simplex virus genome comprising deletions of the coding sequences of ICP4 (both copies) and ICP22, with an expression cassette containing a nucleic acid encoding a polypeptide integrated at each of the ICP4 loci. FIG. 1F shows a modified herpes simplex virus genome comprising deletions of the coding sequences of ICP4 (both copies) and ICP22, with an expression cassette containing a nucleic acid encoding a polypeptide integrated at the ICP22 locus. FIG. 1G shows a modified herpes simplex virus genome comprising deletions of the coding sequences of ICP4 (both copies), UL41, and ICP22, with an expression cassette containing a nucleic acid encoding a polypeptide integrated at each of the ICP4 loci. FIG. 1H shows a modified herpes simplex virus genome comprising deletions of the coding sequences of ICP4 (both copies), UL41, and ICP22, with an expression cassette containing a nucleic acid encoding a polypeptide integrated at the UL41 locus. FIG. 1I shows a modified herpes simplex virus genome comprising deletions of the coding sequences of ICP4 (both copies), UL41, and ICP22, with an expression cassette containing a nucleic acid encoding a polypeptide integrated at the ICP22 locus.

FIG. 2 depicts vector genome quantitation via qPCR in murine plasma samples harvested 24 hours after intracameral, suprachoroidal, intravitreal, or subretinal HSV-mCherry administration. Data are presented as mean±standard error of the mean (SEM), n=3 per group.

FIGS. 3A-3F show histology by H&E staining (FIG. 3A) and the in vivo expression of a fluorescent reporter 24 hours after intracameral administration of HSV-mCherry (FIGS. 3B-3F). mCherry (red) was co-stained with DAPI (nuclei marker; blue), and/or N-cadherin (corneal endothelial cell marker; green).

FIGS. 4A-4G depict the in vivo expression of a fluorescent reporter 24 hours after suprachoroidal administration of HSV-mCherry (FIGS. 4A-4F) and histology by H&E staining (FIG. 4G). mCherry (red) was co-stained with DAPI (nuclei marker; blue), rhodopsin (RHO; photoreceptor marker; green), and/or isomerophydrolase/RPE65 (retinal pigment epithelial cell (RPEC) marker; green).

FIGS. 5A-5F show the in vivo expression of a fluorescent reporter 24 hours after intravitreal administration of HSV-mCherry (FIGS. 5A-5E) and histology by H&E staining (FIG. 5F). mCherry (red) was co-stained with DAPI (nuclei marker; blue), and/or N-cadherin (corneal endothelial cell marker; green).

FIGS. 6A-6G depict the in vivo expression of a fluorescent reporter 24 hours after subretinal administration of HSV-mCherry (FIGS. 6A-6F) and histology by H&E staining (FIG. 6G). mCherry (red) was co-stained with DAPI (nuclei marker; blue), rhodopsin (RHO; photoreceptor marker; green), and/or isomerophydrolase/RPE65 (retinal pigment epithelial cell (RPEC) marker; green).

FIGS. 7A-7D show genome (qPCR) and transcript (qTR-PCR) levels in primary corneal epithelial cells 24 hours post infection (hpi) (FIGS. 7A-7B) or 48 hours hpi (FIGS. 7C-7D) with a modified herpes simplex virus encoding proNGF (HSV-proNGF; FIGS. 7A & 7C) or beta-NGF (HSV-sNGF; FIGS. 7B-7D) transgene at a multiplicity of infection (MOI) of 1 or 4. Data indicative of cells assayed in duplicate, and data are presented as mean±standard error of the mean (SEM), n=1.

FIGS. 8A-8B depict western blot detection of human beta-NGF in uninfected control cell media (mock) or primary corneal epithelial cell media infected with a modified herpes simplex virus encoding proNGF (HSV-proNGF) transgene at a multiplicity of infection (MOI) of 1 or 4. Recombinant human beta-NGF was used as a positive control. FIGS. 8A-8B: lane 1 recombinant human beta-NGF; lane 2 ladder; lane 3 mock; lane 4 HSV-proNGF MOI 1; lane 5 HSV-proNGF MOI 4. FIG. 8A: 8-10 ng of total protein was loaded in lanes 4-5. FIG. 8B: 115-140 ng of total protein was loaded in lanes 4-5 from concentrated infected primary corneal epithelial cell media.

FIGS. 9A-9B show secreted human beta-NGF from primary corneal epithelial cells 48 hours post infection (hpi) with a modified herpes simplex virus encoding proNGF (HSV-proNGF; FIG. 9A) or beta-NGF (HSV-sNGF; FIG. 9B) transgene at a multiplicity of infection (MOI) of 1 or 4 analyzed by ELISA. Data indicative of cells assayed in duplicate, and data are presented as mean±standard error of the mean (SEM), n=1.

FIGS. 10A-10B depict viability of primary corneal epithelial cells 24 hours post infection (hpi) or 48 hours hpi with a modified herpes simplex virus encoding proNGF (HSV-proNGF; FIG. 10A) or beta-NGF (HSV-sNGF; FIG. 10B) transgene at a multiplicity of infection (MOI) of 0, 0.5, 1, or 3 analyzed by Mosmann's Tetrazolium Toxicity (MTT). Data indicative of cells assayed in triplicate, and data are presented as mean±standard error of the mean (SEM), n=1.

FIGS. 11A-11B show cell proliferation (analyzed by a Cell Titer assay) in TF-1 cells 72 hours post treatment with varying concentrations of beta-NGF derived from HEK-293T cells infected with (MOI of 4) a modified herpes simplex virus encoding proNGF (HSV-proNGF; FIG. 11A) or beta-NGF (HSV-sNGF; FIG. 11B) transgene. Media alone and recombinant human beta-NGF served as controls. Data indicative of cells assayed in triplicate, and data are presented as mean±standard error of the mean (SEM), n=1.

FIGS. 12A-12C depict corneal fluorescein staining of eyes immediately after corneal wounding (FIG. 12A 1 mm mechanical corneal wound; FIG. 12B 1 mm deeper mechanical corneal wound penetrating into stroma; FIG. 12C Cross hatch needle scratch wound).

FIGS. 13A-13C show histology by H&E staining 24 hours after corneal wounding and HSV-mCherry infection (FIG. 13A 1 mm mechanical corneal wound; FIG. 13B 1 mm deeper mechanical corneal wound; FIG. 13C Cross hatch needle scratch wound).

FIGS. 14A-14D depict genome (qPCR; FIGS. 14A & 14C) and transcript (qRT-PCR; FIGS. 14B & 14D) in corneal epithelium (FIGS. 14A-14B) or whole eye minus the corneal epithelium (FIGS. 14C-14D) 24 hours after topical application of HSV-mCherry to wounded eyes. Data are presented as mean±standard error of the mean (SEM), n=3 eyes per group.

FIGS. 15A-15C show the in vivo expression of a fluorescent reporter 24 hours after corneal wounding and subsequent topical administration of HSV-mCherry (FIG. 15A 1 mm mechanical corneal wound; FIG. 15B 1 mm deeper mechanical corneal wound; FIG. 15C Cross hatch needle scratch wound). mCherry (red) was co-stained with DAPI (nuclei marker; blue).

FIG. 16 depicts HSV-ABCA4 transduction and ABCA4 transgene expression in transduced healthy human ARPE-19 cells (primary retinal pigment epithelial cells). Data are presented as the average of duplicates±standard error of the mean (SEM).

FIG. 17 shows dose-dependent (multiplicity of infection (MOI) of 0 (mock), 0.05, 0.1, 0.5, 1, or 3) ABCA4 protein expression in HSV-ABCA4 transduced healthy human ARPE-19 cells.

FIGS. 18A-18B depict dose-dependent cytotoxicity assessments in HSV-ABCA4 transduced healthy human ARPE-19 cells at 24 (FIG. 18A) or 48 (FIG. 18B) hours post infection, analyzed by Mosmann's Tetrazolium Toxicity (MTT). Data are presented as the average of triplicates±standard error of the mean (SEM).

FIGS. 19A-19C depict human ABCA4 expression analysis in eyes harvested from vehicle control (FIG. 19A) and HSV-ABCA4 (FIG. 19B) treated Abca4^{-/-} mice 24 hours post suprachoroidal injection, as compared to endogenous ABCA4 in wild-type rodent eyes (FIG. 19C). Representative images of immunofluorescence (IF) staining of ABCA4 in rodent eyes. Two HSV-ABCA4 treated mice are shown. Panels were imaged at 20× magnification. Nuclei were visualized via DAPI staining. ONL=outer nuclear layer.

FIGS. 20A-20B show co-immunofluorescence staining of eye sections from vehicle control (FIG. 20A) and HSV-ABCA4 (FIG. 20B) treated Abca4^{-/-} mice 24 hours post suprachoroidal injection. Representative images of IF staining of human ABCA4 (red) and murine rhodopsin (RHO) (green) in Abca4^{-/-} mouse eyes. Panels were images at 20× magnification. Nuclei were visualized via DAPI staining (blue). ONL=outer nuclear layer.

FIGS. 21A-21H depict histology of eye sections from vehicle control (FIGS. 21 A & 21 E) and HSV-ABCA4 (FIGS. 21B-21D and 21F-21H) treated Abca4−/− mice 24 hours post suprachoroidal injection. Representative images of hematoxylin and eosin (H&E) stained mouse eyes. FIGS. 21A-21D were imaged at 5× magnification and FIGS. 21E-21H were imaged at 20× magnification.

FIGS. 22A-22C: slit-lamp pictures of the eye before surgery (FIG. 22A) and at 8 months after surgery and ophthalmic HSV-COL7 treatment (FIG. 22B). FIG. 22C shows anterior segment optical coherence tomography (OCT) images of the corneal epithelium.

FIG. 23 depicts the serial assessment of visual acuity in a patient's right eye following topical application of HSV-COL7.

### DETAILED DESCRIPTION

The following description sets forth exemplary methods, parameters, and the like. It should be recognized, however, that such a description is not intended as a limitation on the scope of the present disclosure but is instead provided as a description of exemplary embodiments.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Gene Transfer Vectors for Mammalian Cells (J. M. Miller and M. P. Calos, eds., 1987); PCR: The Polymerase Chain Reaction, (Mullis et al., eds., 1994); Short Protocols in Molecular Biology (Wiley and Sons, 1999).

### II. Definitions

Before describing the present disclosure in detail, it is to be understood that the present disclosure is not limited to particular compositions or biological systems, which can, of course, vary. It is also to be understood that the terminology used herein is for the purpose of describing particular embodiments only and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

As used herein, the term “and/or” may include any and all combinations of one or more of the associated listed items. For example, the term “a and/or b” may refer to “a alone”, “b alone”, “a or b”, or “a and b”; the term “a, b, and/or c” may refer to “a alone”, “b alone”, “c alone”, “a or b”, “a or c”, “b or c”, “a, b, or c”, “a and b”, “a and c”, “b and c”, or “a, b, and c”; etc.

As used herein, the term “about” refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising”, “consisting”, and “consisting essentially of” aspects and embodiments.

As used herein, the terms “polynucleotide”, “nucleic acid sequence”, “nucleic acid”, and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

As used herein, a nucleic acid is “operatively linked” or “operably linked” when it is placed into a functional relationship with another nucleic acid sequence. For example, a promoter or enhancer is operably linked to a coding sequence if it affects the transcription of the sequence, or a ribosome binding site is operably linked to a coding sequence if it is positioned so as to facilitate translation. Generally, “operatively linked” or “operably linked” means that the DNA or RNA sequences being linked are contiguous.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of effecting expression of such nucleic acids. Thus, an expression vector may refer to a DNA or RNA construct, such as a plasmid, a phage, recombinant virus, or other vector that, upon introduction into an appropriate host cell, results in expression of the nucleic acids. Appropriate expression vectors are well known to those of skill in the art and include those that are replicable in eukaryotic cells and those that remain episomal or those which integrate into the host cell genome.

As used herein, an “open reading frame” or “ORF” refers to a continuous stretch of nucleic acids, either DNA or RNA, that encode a protein or polypeptide. Typically, the nucleic acids comprise a translation start signal or initiation codon, such as ATG or AUG, and a termination codon.

As used herein, an “untranslated region” or “UTR” refers to untranslated nucleic acids at the 5′ and/or 3′ ends of an open reading frame. The inclusion of one or more UTRs in a polynucleotide may affect post-transcriptional regulation, mRNA stability, and/or translation of the polynucleotide.

As used herein, the term “transgene” refers to a polynucleotide that is capable of being transcribed into RNA and translated and/or expressed under appropriate conditions after being introduced into a cell. In some aspects, it confers a desired property to a cell into which it was introduced, or otherwise leads to a desired therapeutic or diagnostic outcome.

As used herein, the terms “polypeptide,” “protein,” and “peptide” are used interchangeably and may refer to a polymer of two or more amino acids.

As used herein, a “subject”, “host”, or an “individual” refers to any animal classified as a mammal, including humans, domestic and farm animals, and zoo, sports, or pet animals, such as dogs, horses, cats, cows, as well as animals used in research, such as mice, rats, hamsters, rabbits, and non-human primates, etc. In some embodiments, the mammal is human.

As used herein, the terms “pharmaceutical formulation” or “pharmaceutical composition” refer to a preparation which is in such a form as to permit the biological activity of the active ingredient(s) to be effective, and which contains no additional components which are unacceptably toxic to a subject to which the composition or formulation would be administered. “Pharmaceutically acceptable” excipients (e.g., vehicles, additives) are those which can reasonably be administered to a subject mammal to provide an effective dose of the active ingredient(s) employed.

As used herein, an “effective amount” is at least the minimum amount required to affect a measurable improvement or prevention of one or more symptoms of a particular disorder. An “effective amount” may vary according to factors such as the disease state, age, sex, and weight of the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications used to treat symptoms of the disease, delaying the progression of the disease, and/or prolonging survival. An effective amount can be administered in one or more administrations. For purposes of the present disclosure, an effective amount of a recombinant nucleic acid, virus, and/or pharmaceutical composition is an amount sufficient to accomplish prophylactic or therapeutic treatment either directly or indirectly. As is understood in the clinical context, an effective amount of a recombinant nucleic acid, virus, and/or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “treatment” refers to clinical intervention designed to alter the natural course of the individual or cell being treated during the course of clinical pathology. Desirable effects of treatment include decreasing the rate of disease/disorder/defect progression, ameliorating, or palliating the disease/disorder/defect state, and remission or improved prognosis.

As used herein, the term “delaying progression of” a disease/disorder/defect refers to deferring, hindering, slowing, retarding, stabilizing, and/or postponing development of the disease/disorder/defect. This delay can be of varying lengths or time, depending on the history of the disease/disorder/defect and/or the individual being treated. As is evident to one of ordinary skill in the art, a sufficient or significant delay can, in effect, encompass prevention, in that the individual does not develop the disease.

### III. Recombinant Nucleic Acids

Certain aspects of the present disclosure relate to recombinant nucleic acids (e.g., isolated recombinant nucleic acids) comprising one or more (e.g., one or more, two or more, three or more, four or more, five or more, ten or more, etc.) polynucleotides encoding a polypeptide. In some embodiments, the recombinant nucleic acid comprises one polynucleotide encoding a polypeptide. In some embodiments, the recombinant nucleic acid comprises two polynucleotides encoding a polypeptide. In some embodiments, the recombinant nucleic acid comprises three or more polynucleotides encoding a polypeptide. In some embodiments, the recombinant nucleic acid comprises one or more polynucleotides encoding two or more polypeptides. In some embodiments, the recombinant nucleic acid comprises two or more polynucleotides encoding two or more polypeptides. In some embodiments, the two or more polypeptides are identical. In some embodiments, the two or more polypeptides are different.

In some embodiments, the present disclosure relates to recombinant nucleic acids comprising a polynucleotide encoding a chimeric polypeptide comprising: a first polypeptide, a linker polypeptide, and a second polypeptide. In some embodiments, the first and second polypeptides are the same. In some embodiments, the first and second polypeptides are different. In some embodiments, the linker polypeptide is a cleavable linker polypeptide. In some embodiments, the linker polypeptide is a non-cleavable linker polypeptide.

In some embodiments, the recombinant nucleic acid is a vector. In some embodiments, the recombinant nucleic acid is a viral vector. In some embodiments, the recombinant nucleic acid is a herpes viral vector. In some embodiments, the recombinant nucleic acid is a herpes simplex virus amplicon. In some embodiments, the recombinant nucleic acid is a recombinant herpes virus genome. In some embodiments, the recombinant herpes virus genome is a recombinant herpes simplex virus genome. In some embodiments, the recombinant herpes simplex virus genome is a recombinant herpes simplex virus type 1 (HSV-1) genome.

### Polynucleotides Encoding Polypeptides

In some embodiments, the present disclosure relates to recombinant nucleic acids comprising one or more polynucleotides encoding one or more polypeptides (e.g., one or more human polypeptides). Any suitable polypeptide described herein or known in the art may be encoded by a polynucleotide of the present disclosure, including, for example, human polypeptides.

In some embodiments, a polynucleotide of the present disclosure comprises the wild-type coding sequence of any gene described herein or known in the art (including any isoform thereof). In some embodiments, a polynucleotide of the present disclosure comprises a codon-optimized variant of the wild-type coding sequence of any gene described herein or known in the art. In some embodiments, use a of a codon-optimized variant of the coding sequence of a gene increases stability and/or yield of heterologous expression (RNA and/or protein) of the encoded polypeptide in a target cell, as compared to the stability and/or yield of heterologous expression of a corresponding, non-codon-optimized, wild-type sequence. Any suitable method known in the art for performing codon optimization of a sequence for expression in one or more target cells (e.g., one or more human cells) may be used, including, for example, by the methods described by Fath et al. (PLOS One. 2011 Mar. 3; 6 (3): e17596).

In some embodiments, the present disclosure relates to a recombinant nucleic acid comprising one or more polynucleotides comprising the coding sequence of a human gene. Any suitable human gene (including any isoform thereof) known in the art may be encoded by a polynucleotide of the present disclosure, including, for example a SAMD11 gene (see e.g., NCBI Gene ID: 148398; SEQ ID NO: 378), a NPHP4 gene (see e.g., NCBI Gene ID: 261734; SEQ ID NO: 379), a ESPN gene (see e.g., NCBI Gene ID: 83715; SEQ ID NO: 380), a NMNAT1 gene (see e.g., NCBI Gene ID: 64802; SEQ ID NO: 381), a MFN2 gene (see e.g., NCBI Gene ID: 9927; SEQ ID NO: 382), a EMC1 gene (see e.g., NCBI Gene ID: 23065; SEQ ID NO: 383), a PLA2G5 gene (see e.g., NCBI Gene ID: 5322; SEQ ID NO: 384), a DHDDS gene (see e.g., NCBI Gene ID: 79947; SEQ ID NO: 385), a PPT1 gene (see e.g., NCBI Gene ID: 5538; SEQ ID NO: 386), a ELOVL1 gene (see e.g., NCBI Gene ID: 64834; SEQ ID NO: 387), a POMGNT1 gene (see e.g., NCBI Gene ID: 55624; SEQ ID NO: 388), a RPE65 gene (see e.g., NCBI Gene ID: 6121; SEQ ID NO: 389), a ABCA4 gene (see e.g., NCBI Gene ID: 24; SEQ ID NO: 390), a COL11A1 gene (see e.g., NCBI Gene ID: 1301; SEQ ID NO: 391), a GNAT2 gene (see e.g., NCBI Gene ID: 2780; SEQ ID NO: 392), a CLCC1 gene (see e.g., NCBI Gene ID: 23155; SEQ ID NO: 393), a DRAM2 gene (see e.g., NCBI Gene ID: 128338; SEQ ID NO: 394), a PRPF3 gene (see e.g., NCBI Gene ID: 9129; SEQ ID NO: 395), a ENSA gene (see e.g., NCBI Gene ID: 2029; SEQ ID NO: 396), a SEMA4A gene (see e.g., NCBI Gene ID: 64218; SEQ ID NO: 397), a ATF6 gene (see e.g., NCBI Gene ID: 22926; SEQ ID NO: 398), a HMCN1 gene (see e.g., NCBI Gene ID: 83872; SEQ ID NO: 399), a CFH gene (see e.g., NCBI Gene ID: 3075; SEQ ID NO: 400), a CRB1 gene (see e.g., NCBI Gene ID: 23418; SEQ ID NO: 401), a ADIPOR1 gene (see e.g., NCBI Gene ID: 51094; SEQ ID NO: 402), a RD3 gene (see e.g., NCBI Gene ID: 343035; SEQ ID NO: 403), a NEK2 gene (see e.g., NCBI Gene ID: 4751; SEQ ID NO: 404), a FLVCR1 gene (see e.g., NCBI Gene ID: 28982; SEQ ID NO: 405), a USH2A gene (see e.g., NCBI Gene ID: 7399; SEQ ID NO: 406), a SDCCAG8 gene (see e.g., NCBI Gene ID: 10806; SEQ ID NO: 407), a OR2W3 gene (see e.g., NCBI Gene ID: 343171; SEQ ID NO: 408), a NBAS gene (see e.g., NCBI Gene ID: 51594; SEQ ID NO: 409), a AGBL5 gene (see e.g., NCBI Gene ID: 60509; SEQ ID NO: 410), a ZNF513 gene (see e.g., NCBI Gene ID: 130557; SEQ ID NO: 411), a IFT172 gene (see e.g., NCBI Gene ID: 26160; SEQ ID NO: 412), a PCARE gene (see e.g., NCBI Gene ID: 388939; SEQ ID NO: 413), a EFEMP1/TLE5 gene (see e.g., NCBI Gene ID: 2202; SEQ ID NO: 414), a FAM161A gene (see e.g., NCBI Gene ID: 84140; SEQ ID NO: 415), a WDPCP gene (see e.g., NCBI Gene ID: 51057; SEQ ID NO: 416), a ALMS1 gene (see e.g., NCBI Gene ID: 7840; SEQ ID NO: 417), a SNRNP200 gene (see e.g., NCBI Gene ID: 23020; SEQ ID NO: 418), a CNNM4 gene (see e.g., NCBI Gene ID: 26504; SEQ ID NO: 419), a CNGA3 gene (see e.g., NCBI Gene ID: 1261; SEQ ID NO: 420), a NPHP1 gene (see e.g., NCBI Gene ID: 4867; SEQ ID NO: 421), a MERTK gene (see e.g., NCBI Gene ID: 10461; SEQ ID NO: 422), a BBS5 gene (see e.g., NCBI Gene ID: 129880; SEQ ID NO: 423), a CERKL gene (see e.g., NCBI Gene ID: 375298; SEQ ID NO: 424), a NEUROD1 gene (see e.g., NCBI Gene ID: 4760; SEQ ID NO: 425), a TMEM237 gene (see e.g., NCBI Gene ID: 65062; SEQ ID NO: 426), a KCNJ13 gene (see e.g., NCBI Gene ID: 3769; SEQ ID NO: 427), a SAG gene (see e.g., NCBI Gene ID: 6295; SEQ ID NO: 428), a SPP2 gene (see e.g., NCBI Gene ID: 6694; SEQ ID NO: 429), a TRNT1 gene (see e.g., NCBI Gene ID: 51095; SEQ ID NO: 430), a SLC4A7 gene (see e.g., NCBI Gene ID: 9497; SEQ ID NO: 431), a LZTFL1 gene (see e.g., NCBI Gene ID: 54585; SEQ ID NO: 432), a GNAT1 gene (see e.g., NCBI Gene ID: 2779; SEQ ID NO: 433), a TREX1 gene (see e.g., NCBI Gene ID: 11277; SEQ ID NO: 434), a MAPKAPK3 gene (see e.g., NCBI Gene ID: 7867; SEQ ID NO: 435), a ATXN7 gene (see e.g., NCBI Gene ID: 6314; SEQ ID NO: 436), a PROS1 gene (see e.g., NCBI Gene ID: 5627; SEQ ID NO: 437), a ARL6 gene (see e.g., NCBI Gene ID: 84100; SEQ ID NO: 438), a IMPG2 gene (see e.g., NCBI Gene ID: 50939; SEQ ID NO: 439), a IQCB1 gene (see e.g., NCBI Gene ID: 9657; SEQ ID NO: 440), a RHO gene (see e.g., NCBI Gene ID: 6010; SEQ ID NO: 441), a NPHP3 gene (see e.g., NCBI Gene ID: 27031; SEQ ID NO: 442), a CLRN1 gene (see e.g., NCBI Gene ID: 7401; SEQ ID NO: 443), a SLC7A14 gene (see e.g., NCBI Gene ID: 57709; SEQ ID NO: 444), a PCYT1A gene (see e.g., NCBI Gene ID: 5130; SEQ ID NO: 445), a CEP19 gene (see e.g., NCBI Gene ID: 84984; SEQ ID NO: 446), a PDE6B gene (see e.g., NCBI Gene ID: 5158; SEQ ID NO: 447), a WFS1 gene (see e.g., NCBI Gene ID: 7466; SEQ ID NO: 448), a HMX1 gene (see e.g., NCBI Gene ID: 3166; SEQ ID NO: 449), a RAB28 gene (see e.g., NCBI Gene ID: 9364; SEQ ID NO: 450), a CC2D2A gene (see e.g., NCBI Gene ID: 57545; SEQ ID NO: 451), a PROM1 gene (see e.g., NCBI Gene ID: 8842; SEQ ID NO: 452), a ADGRA3 gene (see e.g., NCBI Gene ID: 166647; SEQ ID NO: 453), a DTHD1 gene (see e.g., NCBI Gene ID: 401124; SEQ ID NO: 454), a WDR19 gene (see e.g., NCBI Gene ID: 57728; SEQ ID NO: 455), a CNGA1 gene (see e.g., NCBI Gene ID: 1259; SEQ ID NO: 456), a CISD2 gene (see e.g., NCBI Gene ID: 493856; SEQ ID NO: 457), a MTTP gene (see e.g., NCBI Gene ID: 4547; SEQ ID NO: 458), a LRIT3 gene (see e.g., NCBI Gene ID: 345193; SEQ ID NO: 459), a BBS7 gene (see e.g., NCBI Gene ID: 55212; SEQ ID NO: 460), a BBS12 gene (see e.g., NCBI Gene ID: 166379; SEQ ID NO: 461), a MFSD8 gene (see e.g., NCBI Gene ID: 256471; SEQ ID NO: 462), a PLK4 gene (see e.g., NCBI Gene ID: 10733; SEQ ID NO: 463), a LRAT gene (see e.g., NCBI Gene ID: 9227; SEQ ID NO: 464), a TLR3 gene (see e.g., NCBI Gene ID: 7098; SEQ ID NO: 465), a CYP4V2 gene (see e.g., NCBI Gene ID: 285440; SEQ ID NO: 466), a CWC27 gene (see e.g., NCBI Gene ID: 10283; SEQ ID NO: 467), a POC5 gene (see e.g., NCBI Gene ID: 134359; SEQ ID NO: 468), a VCAN gene (see e.g., NCBI Gene ID: 1462; SEQ ID NO: 469), a ADGRV1 gene (see e.g., NCBI Gene ID: 84059; SEQ ID NO: 470), a NR2F1 gene (see e.g., NCBI Gene ID: 7025; SEQ ID NO: 471), a SLC25A46 gene (see e.g., NCBI Gene ID: 91137; SEQ ID NO: 472), a CTNNA1 gene (see e.g., NCBI Gene ID: 1495; SEQ ID NO: 473), a HARS1 gene (see e.g., NCBI Gene ID: 3035; SEQ ID NO: 474), a PDE6A gene (see e.g., NCBI Gene ID: 5145; SEQ ID NO: 475), a GRM6 gene (see e.g., NCBI Gene ID: 2916; SEQ ID NO: 476), a MAK gene (see e.g., NCBI Gene ID: 4117; SEQ ID NO: 477), a C2 gene (see e.g., NCBI Gene ID: 717; SEQ ID NO: 478), a CFB gene (see e.g., NCBI Gene ID: 629; SEQ ID NO: 479), a TULP1 gene (see e.g., NCBI Gene ID: 7287; SEQ ID NO: 480), a GUCA1A gene (see e.g., NCBI Gene ID: 2978; SEQ ID NO: 481), a GUCA1B gene (see e.g., NCBI Gene ID: 2979; SEQ ID NO: 482), a PRPH2 gene (see e.g., NCBI Gene ID: 5961; SEQ ID NO: 483), a IMPG1 gene (see e.g., NCBI Gene ID: 3617; SEQ ID NO: 484), a EYS gene (see e.g., NCBI Gene ID: 346007; SEQ ID NO: 485), a COL9A1 gene (see e.g., NCBI Gene ID: 1297; SEQ ID NO: 486), a RIMS1 gene (see e.g., NCBI Gene ID: 22999; SEQ ID NO: 487), a LCA5 gene (see e.g., NCBI Gene ID: 167691; SEQ ID NO: 488), a ELOVL4 gene (see e.g., NCBI Gene ID: 6785; SEQ ID NO: 489), a PRDM13 gene (see e.g., NCBI Gene ID: 59336; SEQ ID NO: 490), a RTN4IP1 gene (see e.g., NCBI Gene ID: 84816; SEQ ID NO: 491), a AHI1 gene (see e.g., NCBI Gene ID: 54806; SEQ ID NO: 492), a PEX7 gene (see e.g., NCBI Gene ID: 5191; SEQ ID NO: 493), a CNOT9 gene (see e.g., NCBI Gene ID: 9125; SEQ ID NO: 494), a AHR gene (see e.g., NCBI Gene ID: 196; SEQ ID NO: 495), a KLHL7 gene (see e.g., NCBI Gene ID: 55975; SEQ ID NO: 496), a RP9 gene (see e.g., NCBI Gene ID: 6100; SEQ ID NO: 497), a BBS9 gene (see e.g., NCBI Gene ID: 27241; SEQ ID NO: 498), a PEX1 gene (see e.g., NCBI Gene ID: 5189; SEQ ID NO: 499), a TSPAN12 gene (see e.g., NCBI Gene ID: 23554; SEQ ID NO: 500), a IMPDH1 gene (see e.g., NCBI Gene ID: 3614; SEQ ID NO: 501), a OPN1SW gene (see e.g., NCBI Gene ID: 611; SEQ ID NO: 502), a KIAA1549 gene (see e.g., NCBI Gene ID: 57670; SEQ ID NO: 503), a RP1L1 gene (see e.g., NCBI Gene ID: 94137; SEQ ID NO: 504), a ADAM9 gene (see e.g., NCBI Gene ID: 8754; SEQ ID NO: 505), a HGSNAT gene (see e.g., NCBI Gene ID: 138050; SEQ ID NO: 506), a RP1 gene (see e.g., NCBI Gene ID: 6101; SEQ ID NO: 507), a TTPA gene (see e.g., NCBI Gene ID: 7274; SEQ ID NO: 508), a CSPP1 gene (see e.g., NCBI Gene ID: 79848; SEQ ID NO: 509), a OPA1 gene (see e.g., NCBI Gene ID: 4976; SEQ ID NO: 510), a PEX2 gene (see e.g., NCBI Gene ID: 5828; SEQ ID NO: 511), a CNGB3 gene (see e.g., NCBI Gene ID: 54714; SEQ ID NO: 512), a CFAP418 gene (see e.g., NCBI Gene ID: 157657; SEQ ID NO: 513), a GDF6 gene (see e.g., NCBI Gene ID: 392255; SEQ ID NO: 514), a RIMS2 gene (see e.g., NCBI Gene ID: 9699; SEQ ID NO: 515), a KCNV2 gene (see e.g., NCBI Gene ID: 169522; SEQ ID NO: 516), a TOPORS gene (see e.g., NCBI Gene ID: 10210; SEQ ID NO: 517), a CEP78 gene (see e.g., NCBI Gene ID: 84131; SEQ ID NO: 518), a INVS gene (see e.g., NCBI Gene ID: 27130; SEQ ID NO: 519), a PRPF4 gene (see e.g., NCBI Gene ID: 9128; SEQ ID NO: 520), a WHRN gene (see e.g., NCBI Gene ID: 25861; SEQ ID NO: 521), a TRIM32 gene (see e.g., NCBI Gene ID: 22954; SEQ ID NO: 522), a TLR4 gene (see e.g., NCBI Gene ID: 7099; SEQ ID NO: 523), a DYNC212 gene (see e.g., NCBI Gene ID: 89891; SEQ ID NO: 524), a PDCD2 gene (see e.g., NCBI Gene ID: 5134; SEQ ID NO: 525), a EXOSC2 gene (see e.g., NCBI Gene ID: 23404; SEQ ID NO: 526), a INPP5E gene (see e.g., NCBI Gene ID: 56623; SEQ ID NO: 527), a PHYH gene (see e.g., NCBI Gene ID: 5264; SEQ ID NO: 528), a ACBD5 gene (see e.g., NCBI Gene ID: 91452; SEQ ID NO: 529), a PCDH15 gene (see e.g., NCBI Gene ID: 65217; SEQ ID NO: 530), a RBP3 gene (see e.g., NCBI Gene ID: 5949; SEQ ID NO: 531), a ERCC6 gene (see e.g., NCBI Gene ID: 2074; SEQ ID NO: 532), a HK1 gene (see e.g., NCBI Gene ID: 3098; SEQ ID NO: 533), a CDH23 gene (see e.g., NCBI Gene ID: 64072; SEQ ID NO: 534), a CDHR1 gene (see e.g., NCBI Gene ID: 92211; SEQ ID NO: 535), a RGR gene (see e.g., NCBI Gene ID: 5995; SEQ ID NO: 536), a KIF11 gene (see e.g., NCBI Gene ID: 3832; SEQ ID NO: 537), a RBP4 gene (see e.g., NCBI Gene ID: 5950; SEQ ID NO: 538), a PDE6C gene (see e.g., NCBI Gene ID: 5146; SEQ ID NO: 539), a PAX2 gene (see e.g., NCBI Gene ID: 5076; SEQ ID NO: 540), a PDZD7 gene (see e.g., NCBI Gene ID: 79955; SEQ ID NO: 541), a ARL3 gene (see e.g., NCBI Gene ID: 403; SEQ ID NO: 542), a BBIP1 gene (see e.g., NCBI Gene ID: 92482; SEQ ID NO: 543), a ARMS2 gene (see e.g., NCBI Gene ID: 387715; SEQ ID NO: 544), a HTRA1 gene (see e.g., NCBI Gene ID: 5654; SEQ ID NO: 545), a OAT gene (see e.g., NCBI Gene ID: 4942; SEQ ID NO: 546), a ZNF408 gene (see e.g., NCBI Gene ID: 79797; SEQ ID NO: 547), a TUB gene (see e.g., NCBI Gene ID: 7275; SEQ ID NO: 548), a TEAD1 gene (see e.g., NCBI Gene ID: 7003; SEQ ID NO: 549), a USH1C gene (see e.g., NCBI Gene ID: 10083; SEQ ID NO: 550), a TMEM216 gene (see e.g., NCBI Gene ID: 51259; SEQ ID NO: 551), a BEST1 gene (see e.g., NCBI Gene ID: 7439; SEQ ID NO: 552), a ASRGL1 gene (see e.g., NCBI Gene ID: 80150; SEQ ID NO: 553), a ROM1 gene (see e.g., NCBI Gene ID: 6094; SEQ ID NO: 554), a BBS1 gene (see e.g., NCBI Gene ID: 582; SEQ ID NO: 555), a CABP4 gene (see e.g., NCBI Gene ID: 57010; SEQ ID NO: 556), a LRP5 gene (see e.g., NCBI Gene ID: 4041; SEQ ID NO: 557), a CAPN5 gene (see e.g., NCBI Gene ID: 726; SEQ ID NO: 558), a MYO7A gene (see e.g., NCBI Gene ID: 4647; SEQ ID NO: 559), a TMEM126A gene (see e.g., NCBI Gene ID: 84233; SEQ ID NO: 560), a FZD4 gene (see e.g., NCBI Gene ID: 8322; SEQ ID NO: 561), a DYNC2LI1 gene (see e.g., NCBI Gene ID: 51626; SEQ ID NO: 562), a CEP164 gene (see e.g., NCBI Gene ID: 22897; SEQ ID NO: 563), a C1QTNF5 gene (see e.g., NCBI Gene ID: 114902; SEQ ID NO: 564), a MFRP gene (see e.g., NCBI Gene ID: 83552; SEQ ID NO: 565), a CACNA2D4 gene (see e.g., NCBI Gene ID: 93589; SEQ ID NO: 566), a GNB3 gene (see e.g., NCBI Gene ID: 2784; SEQ ID NO: 567), a PDE6H gene (see e.g., NCBI Gene ID: 5149; SEQ ID NO: 568), a COL2A1 gene (see e.g., NCBI Gene ID: 1280; SEQ ID NO: 569), a MMP19 gene (see e.g., NCBI Gene ID: 4327; SEQ ID NO: 570), a RDH5 gene (see e.g., NCBI Gene ID: 5959; SEQ ID NO: 571), a CCT2 gene (see e.g., NCBI Gene ID: 10576; SEQ ID NO: 572), a BBS10 gene (see e.g., NCBI Gene ID: 79738; SEQ ID NO: 573), a CEP290 gene (see e.g., NCBI Gene ID: 80184; SEQ ID NO: 574), a POC1B gene (see e.g., NCBI Gene ID: 282809; SEQ ID NO: 575), a MVK gene (see e.g., NCBI Gene ID: 4598; SEQ ID NO: 576), a IFT81 gene (see e.g., NCBI Gene ID: 28981; SEQ ID NO: 577), a MTRFR gene (see e.g., NCBI Gene ID: 91574; SEQ ID NO: 578), a ITM2B gene (see e.g., NCBI Gene ID: 9445; SEQ ID NO: 579), a RB1 gene (see e.g., NCBI Gene ID: 5925; SEQ ID NO: 580), a RCBTB1 gene (see e.g., NCBI Gene ID: 55213; SEQ ID NO: 581), a GRK1 gene (see e.g., NCBI Gene ID: 6011; SEQ ID NO: 582), a RPGRIP1 gene (see e.g., NCBI Gene ID: 57096; SEQ ID NO: 583), a NRL gene (see e.g., NCBI Gene ID: 4901; SEQ ID NO: 584), a OTX2 gene (see e.g., NCBI Gene ID: 5015; SEQ ID NO: 585), a RDH11 gene (see e.g., NCBI Gene ID: 51109; SEQ ID NO: 585), a RDH12 gene (see e.g., NCBI Gene ID: 145226; SEQ ID NO: 587), a TTLL5 gene (see e.g., NCBI Gene ID: 23093; SEQ ID NO: 588), a SPATA7 gene (see e.g., NCBI Gene ID: 55812; SEQ ID NO: 589), a TTC8 gene (see e.g., NCBI Gene ID: 123016; SEQ ID NO: 590), a FBLN5 gene (see e.g., NCBI Gene ID: 10516; SEQ ID NO: 591), a TRPM1 gene (see e.g., NCBI Gene ID: 4308; SEQ ID NO: 592), a TUBGCP4 gene (see e.g., NCBI Gene ID: 27229; SEQ ID NO: 593), a SLC24A1 gene (see e.g., NCBI Gene ID: 9187; SEQ ID NO: 594), a NR2E3 gene (see e.g., NCBI Gene ID: 10002; SEQ ID NO: 595), a BBS4 gene (see e.g., NCBI Gene ID: 585; SEQ ID NO: 596), a CIB2 gene (see e.g., NCBI Gene ID: 10518; SEQ ID NO: 597), a RLBP1 gene (see e.g., NCBI Gene ID: 6017; SEQ ID NO: 598), a GNPTG gene (see e.g., NCBI Gene ID: 84572; SEQ ID NO: 599), a IFT140 gene (see e.g., NCBI Gene ID: 9742; SEQ ID NO: 600), a CLUAP1 gene (see e.g., NCBI Gene ID: 23059; SEQ ID NO: 601), a ABCC6 gene (see e.g., NCBI Gene ID: 368; SEQ ID NO: 602), a CRYM gene (see e.g., NCBI Gene ID: 1428; SEQ ID NO: 603), a CLN3 gene (see e.g., NCBI Gene ID: 1201; SEQ ID NO: 604), a ZNF423 gene (see e.g., NCBI Gene ID: 23090; SEQ ID NO: 605), a RPGRIP1L gene (see e.g., NCBI Gene ID: 23322; SEQ ID NO: 606), a BBS2 gene (see e.g., NCBI Gene ID: 583; SEQ ID NO: 607), a ARL2BP gene (see e.g., NCBI Gene ID: 23568; SEQ ID NO: 608), a CNGB1 gene (see e.g., NCBI Gene ID: 1258; SEQ ID NO: 609), a CDH3 gene (see e.g., NCBI Gene ID: 1001; SEQ ID NO: 610), a DHX38 gene (see e.g., NCBI Gene ID: 9785; SEQ ID NO: 611), a ADAMTS18 gene (see e.g., NCBI Gene ID: 170692; SEQ ID NO: 612), a SLC38A8 gene (see e.g., NCBI Gene ID: 146167; SEQ ID NO: 613), a GUCY2D gene (see e.g., NCBI Gene ID: 3000; SEQ ID NO: 614), a PRPF8 gene (see e.g., NCBI Gene ID: 10594; SEQ ID NO: 615), a AIPL1 gene (see e.g., NCBI Gene ID: 23746; SEQ ID NO: 616), a PITPNM3 gene (see e.g., NCBI Gene ID: 83394; SEQ ID NO: 617), a UNC119 gene (see e.g., NCBI Gene ID: 9094; SEQ ID NO: 618), a GPR179 gene (see e.g., NCBI Gene ID: 440435; SEQ ID NO: 619), a MKS1 gene (see e.g., NCBI Gene ID: 54903; SEQ ID NO: 620), a CA4 gene (see e.g., NCBI Gene ID: 762; SEQ ID NO: 621), a RGS9 gene (see e.g., NCBI Gene ID: 8787; SEQ ID NO: 622), a ARSG gene (see e.g., NCBI Gene ID: 22901; SEQ ID NO: 623), a USH1G gene (see e.g., NCBI Gene ID: 124590; SEQ ID NO: 624), a PRCD gene (see e.g., NCBI Gene ID: 768206; SEQ ID NO: 625), a FSCN2 gene (see e.g., NCBI Gene ID: 25794; SEQ ID NO: 626), a PDE6G gene (see e.g., NCBI Gene ID: 5148; SEQ ID NO: 627), a LAMA1 gene (see e.g., NCBI Gene ID: 284217; SEQ ID NO: 628), a AFG3L2 gene (see e.g., NCBI Gene ID: 10939; SEQ ID NO: 629), a CRX gene (see e.g., NCBI Gene ID: 1406; SEQ ID NO: 630), a REEP6 gene (see e.g., NCBI Gene ID: 92840; SEQ ID NO: 631), a RAX2 gene (see e.g., NCBI Gene ID: 84839; SEQ ID NO: 632), a C3 gene (see e.g., NCBI Gene ID: 718; SEQ ID NO: 633), a ARHGEF18 gene (see e.g., NCBI Gene ID: 23370; SEQ ID NO: 634), a PNPLA6 gene (see e.g., NCBI Gene ID: 10908; SEQ ID NO: 635), a RGS9BP gene (see e.g., NCBI Gene ID: 388531; SEQ ID NO: 636), a OPA3 gene (see e.g., NCBI Gene ID: 80207; SEQ ID NO: 637), a PRPF31 gene (see e.g., NCBI Gene ID: 26121; SEQ ID NO: 638), a IDH3B gene (see e.g., NCBI Gene ID: 3420; SEQ ID NO: 639), a PANK2 gene (see e.g., NCBI Gene ID: 80025; SEQ ID NO: 640), a JAG1 gene (see e.g., NCBI Gene ID: 182; SEQ ID NO: 641), a MKKS gene (see e.g., NCBI Gene ID: 8195; SEQ ID NO: 642), a KIZ gene (see e.g., NCBI Gene ID: 55857; SEQ ID NO: 643), a ABHD12 gene (see e.g., NCBI Gene ID: 26090; SEQ ID NO: 644), a KIF3B gene (see e.g., NCBI Gene ID: 9371; SEQ ID NO: 645), a CEP250 gene (see e.g., NCBI Gene ID: 11190; SEQ ID NO: 646), a PRPF6 gene (see e.g., NCBI Gene ID: 24148; SEQ ID NO: 647), a CFAP410 gene (see e.g., NCBI Gene ID: 755; SEQ ID NO: 648), a DNM1L gene (see e.g., NCBI Gene ID: 10059; SEQ ID NO: 649), a TIMP3 gene (see e.g., NCBI Gene ID: 7078; SEQ ID NO: 650), a IFT27 gene (see e.g., NCBI Gene ID: 11020; SEQ ID NO: 651), a FBLN1 gene (see e.g., NCBI Gene ID: 2192; SEQ ID NO: 652), a MIEF1 gene (see e.g., NCBI Gene ID; SEQ ID NO: 653), a ACO2 gene (see e.g., NCBI Gene ID: 50; SEQ ID NO: 654), a TUBGCP6 gene (see e.g., NCBI Gene ID: 85378; SEQ ID NO: 655), a OFD1 gene (see e.g., NCBI Gene ID: 8481; SEQ ID NO: 656), a RS1 gene (see e.g., NCBI Gene ID: 6247; SEQ ID NO: 657), a RP2 gene (see e.g., NCBI Gene ID: 6102; SEQ ID NO: 658), a DMD gene (see e.g., NCBI Gene ID: 1756; SEQ ID NO: 659), a RPGR gene (see e.g., NCBI Gene ID: 6103; SEQ ID NO: 660), a NYX gene (see e.g., NCBI Gene ID: 60506; SEQ ID NO: 661), a PEPD gene (see e.g., NCBI Gene ID: 5184; SEQ ID NO: 662), a NDP gene (see e.g., NCBI Gene ID: 4693; SEQ ID NO: 663), a CACNA1F gene (see e.g., NCBI Gene ID: 778; SEQ ID NO: 664), a PGK1 gene (see e.g., NCBI Gene ID: 5230; SEQ ID NO: 665), a CHM gene (see e.g., NCBI Gene ID: 1121; SEQ ID NO: 666), a TIMM8A gene (see e.g., NCBI Gene ID: 1678; SEQ ID NO: 667), a PRPS1 gene (see e.g., NCBI Gene ID: 5631; SEQ ID NO: 668), a OPN1LW gene (see e.g., NCBI Gene ID: 5956; SEQ ID NO: 669), a OPN1MW gene (see e.g., NCBI Gene ID: 2652; SEQ ID NO: 670), a OPN1SW gene (see e.g., NCBI Gene ID: 611; SEQ ID NO: 671), a TFAM gene (see e.g., NCBI Gene ID: 7019; SEQ ID NO: 672), a MT-ND1 gene (see e.g., NCBI Gene ID: 4535; SEQ ID NO: 673), a MT-ND2 gene (see e.g., NCBI Gene ID: 4536; SEQ ID NO: 674), a MT-ND3 gene (see e.g., NCBI Gene ID: 4537; SEQ ID NO: 675), a MT-ND4L gene (see e.g., NCBI Gene ID: 4539; SEQ ID NO: 676), a MT-ND4 gene (see e.g., NCBI Gene ID: 4538; SEQ ID NO: 677), a MT-ND5 gene (see e.g., NCBI Gene ID: 4540; SEQ ID NO: 678), a MT-ND6 gene (see e.g., NCBI Gene ID: 4541; SEQ ID NO: 679), a MT-ATP6 gene (see e.g., NCBI Gene ID: 4508; SEQ ID NO: 680), a MT-ATP8 gene (see e.g., NCBI Gene ID: 4509; SEQ ID NO: 681), a MT-CO1 gene (see e.g., NCBI Gene ID: 4512; SEQ ID NO: 682), a MT-CO3 gene (see e.g., NCBI Gene ID: 4514; SEQ ID NO: 683), a MT-CYB gene (see e.g., NCBI Gene ID: 4519; SEQ ID NO: 684), a LARS2 gene (see e.g., NCBI Gene ID: 23395; SEQ ID NO: 685), a EARS2 gene (see e.g., NCBI Gene ID: 124454; SEQ ID NO: 686), a KARS1 gene (see e.g., NCBI Gene ID: 3735; SEQ ID NO: 687), a HARS2 gene (see e.g., NCBI Gene ID: 23438; SEQ ID NO: 688), a SARS2 gene (see e.g., NCBI Gene ID: 54938; SEQ ID NO: 689), a PARS2 gene (see e.g., NCBI Gene ID: 25973; SEQ ID NO: 690), a MMACHC gene (see e.g., NCBI Gene ID: 25974; SEQ ID NO: 691), a POU3F4 gene (see e.g., NCBI Gene ID: 5456; SEQ ID NO: 692), a RPS6KA6 gene (see e.g., NCBI Gene ID: 27330; SEQ ID NO: 693), a CPLANE1 gene (see e.g., NCBI Gene ID: 65250; SEQ ID NO: 694), a TMEM67 gene (see e.g., NCBI Gene ID: 91147; SEQ ID NO: 695), a TRAF3IP1 gene (see e.g., NCBI Gene ID: 26146; SEQ ID NO: 696), a IFT74 gene (see e.g., NCBI Gene ID: 80173; SEQ ID NO: 697), a SCAPER gene (see e.g., NCBI Gene ID: 49855; SEQ ID NO: 698), a SCLT1 gene (see e.g., NCBI Gene ID: 132320; SEQ ID NO: 699), a KIAA0586 gene (see e.g., NCBI Gene ID: 9786; SEQ ID NO: 700), a TCTN2 gene (see e.g., NCBI Gene ID: 79867; SEQ ID NO: 701), a ARL13B gene (see e.g., NCBI Gene ID: 200894; SEQ ID NO: 702), a B9D1 gene (see e.g., NCBI Gene ID: 27077; SEQ ID NO: 703), a B9D2 gene (see e.g., NCBI Gene ID: 80776; SEQ ID NO: 704), a C2CD3 gene (see e.g., NCBI Gene ID: 26005; SEQ ID NO: 705), a CEP41 gene (see e.g., NCBI Gene ID: 95681; SEQ ID NO: 706), a CEP104 gene (see e.g., NCBI Gene ID: 9731; SEQ ID NO: 707), a CEP120 gene (see e.g., NCBI Gene ID: 153241; SEQ ID NO: 708), a IFT172 gene (see e.g., NCBI Gene ID: 26160; SEQ ID NO: 709), a KATNIP gene (see e.g., NCBI Gene ID: 23247; SEQ ID NO: 710), a KIF7 gene (see e.g., NCBI Gene ID: 374654; SEQ ID NO: 711), a PDE6D gene (see e.g., NCBI Gene ID: 5147; SEQ ID NO: 712), a TCTN1 gene (see e.g., NCBI Gene ID: 79600; SEQ ID NO: 713), a TCTN3 gene (see e.g., NCBI Gene ID: 26123; SEQ ID NO: 714), a TMEM107 gene (see e.g., NCBI Gene ID: 84314; SEQ ID NO: 715), a TMEM138 gene (see e.g., NCBI Gene ID: 51524; SEQ ID NO: 716), a TMEM231 gene (see e.g., NCBI Gene ID: 79583; SEQ ID NO: 717), a TTC21B gene (see e.g., NCBI Gene ID: 79809; SEQ ID NO: 718), a RORA gene (see e.g., NCBI Gene ID: 6095; SEQ ID NO: 719), a NGF gene (see e.g., NCBI Gene ID: 4803; SEQ ID NO: 720), a COL8A2 gene (see e.g., NCBI Gene ID: 1296; SEQ ID NO: 721), a SLC4A11 gene (see e.g., NCBI Gene ID: 83959; SEQ ID NO: 722), a ZEB1 gene (see e.g., NCBI Gene ID: 6935; SEQ ID NO: 723), a KRT83 gene (see e.g., NCBI Gene ID: 3889; SEQ ID NO: 724), a KRT12 gene (see e.g., NCBI Gene ID: 3859; SEQ ID NO: 725), a TGFBI gene (see e.g., NCBI Gene ID: 7045; SEQ ID NO: 726), a TACSTD2 gene (see e.g., NCBI Gene ID: 4070; SEQ ID NO: 727), a CHST6 gene (see e.g., NCBI Gene ID: 4166; SEQ ID NO: 728), a GSN gene (see e.g., NCBI Gene ID: 2934; SEQ ID NO: 729), a UBIAD1 gene (see e.g., NCBI Gene ID: 29914; SEQ ID NO: 730), a DCN gene (see e.g., NCBI Gene ID: 1634; SEQ ID NO: 731), a PIKFYVE gene (see e.g., NCBI Gene ID: 200576; SEQ ID NO: 732), a OVOL2 gene (see e.g., NCBI Gene ID: 58495; SEQ ID NO: 733), a GRHL2 gene (see e.g., NCBI Gene ID: 79977; SEQ ID NO: 734), etc. In some embodiments, a polynucleotide of the present disclosure comprises a sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of any of the human genes (and/or coding sequences thereof) described herein or known in the art.

In some embodiments, a polynucleotide of the present disclosure encodes a beta-nerve growth factor polypeptide. In some embodiments, the beta-nerve growth factor polypeptide is a human beta-nerve growth factor polypeptide (see e.g., UniProt accession number: P01138). In some embodiments, the polynucleotide comprises the coding sequence of a wild-type NGF gene (see e.g., NCBI Gene ID: 4803, SEQ ID NO: 720), or a codon-optimized variant thereof (see e.g., SEQ ID NO: 735 SEQ ID NO. 736, SEQ ID NO: 741, SEQ ID NO: 742, and/or SEQ ID NO: 743). In some embodiments, a polynucleotide encoding a beta-nerve growth factor polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 363. In some embodiments, a polynucleotide encoding a beta-nerve growth factor polypeptide is a polynucleotide that encodes a polypeptide comprising the amino acid sequence of SEQ ID NO: 363.

In some embodiments, a polynucleotide of the present disclosure encodes a beta-nerve growth factor polypeptide. In some embodiments, the beta-nerve growth factor polypeptide is a human beta-nerve growth factor polypeptide (see e.g., UniProt accession number: P01138). In some embodiments, the polynucleotide comprises the coding sequence of a wild-type NGF gene (see e.g., NCBI Gene ID: 4803, SEQ ID NO: 720), or a codon-optimized variant thereof (see e.g., SEQ ID NO: 735, SEQ ID NO. 736, SEQ ID NO: 741, SEQ ID NO: 742, and/or SEQ ID NO: 743). In some embodiments, a polynucleotide encoding a beta-nerve growth factor polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 739. In some embodiments, a polynucleotide encoding a beta-nerve growth factor polypeptide is a polynucleotide that encodes a polypeptide comprising the amino acid sequence of SEQ ID NO: 739.

In some embodiments, a polynucleotide of the present disclosure encodes a beta-nerve growth factor polypeptide. In some embodiments, the beta-nerve growth factor polypeptide is a human beta-nerve growth factor polypeptide (see e.g., UniProt accession number: P01138). In some embodiments, the polynucleotide comprises the coding sequence of a wild-type NGF gene (see e.g., NCBI Gene ID: 4803, SEQ ID NO: 720), or a codon-optimized variant thereof (see e.g., SEQ ID NO: 735, SEQ ID NO. 736, SEQ ID NO: 741, SEQ ID NO: 742, and/or SEQ ID NO: 743). In some embodiments, a polynucleotide encoding a beta-nerve growth factor polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 740. In some embodiments, a polynucleotide encoding a beta-nerve growth factor polypeptide is a polynucleotide that encodes a polypeptide comprising the amino acid sequence of SEQ ID NO: 740.

In some embodiments, a polynucleotide encoding a beta-nerve growth factor polypeptide is a polynucleotide that encodes an N-terminal truncation, a C-terminal truncation, or a fragment of the amino acid sequence of SEQ ID NO: 363, SEQ ID NO: 739, and/or SEQ ID NO: 740. N-terminal truncations, C-terminal truncations, or fragments may comprise at least 10, at least 12, at least 14, at least 16, at least 18, at least 20, at least 30, at least 40, at least 50, at least 75, at least 100, at least 200, but fewer than 241, consecutive amino acids of SEQ ID NO: 363, SEQ ID NO: 739, and/or SEQ ID NO: 740.

In some embodiments, a polynucleotide of the present disclosure encodes a retinal-specific phospholipid-transporting ATPase polypeptide. In some embodiments, the retinal-specific phospholipid-transporting ATPase polypeptide is a human retinal-specific phospholipid-transporting ATPase polypeptide (see e.g., UniProt accession number: P78363). In some embodiments, the polynucleotide comprises the coding sequence of a wild-type ABCA4 gene (see e.g., NCBI Gene ID: 24, SEQ ID NO: 390), or a codon-optimized variant thereof (see e.g., SEQ ID NO: 737). In some embodiments, a polynucleotide encoding a retinal-specific phospholipid-transporting ATPase polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 33. In some embodiments, a polynucleotide encoding a retinal-specific phospholipid-transporting ATPase polypeptide is a polynucleotide that encodes a polypeptide comprising the amino acid sequence of SEQ ID NO: 33.

In some embodiments, a polynucleotide encoding a retinal-specific phospholipid-transporting ATPase polypeptide is a polynucleotide that encodes an N-terminal truncation, a C-terminal truncation, or a fragment of the amino acid sequence of SEQ ID NO: 33. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1,000, but fewer than 2,273, consecutive amino acids of SEQ ID NO: 33.

In some embodiments, a polynucleotide of the present disclosure encodes a centrosomal protein of 290 kDa polypeptide. In some embodiments, the centrosomal protein of 290 kDa polypeptide is a human centrosomal protein of 290 kDa polypeptide (see e.g., UniProt accession number: 015078). In some embodiments, the polynucleotide comprises the coding sequence of a wild-type CEP290 gene (see e.g., NCBI Gene ID: 80184, SEQ ID NO: 574), or a codon-optimized variant thereof (see e.g., SEQ ID NO: 738). In some embodiments, a polynucleotide encoding a centrosomal protein of 290 kDa polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 217. In some embodiments, a polynucleotide encoding a centrosomal protein of 290 kDa polypeptide is a polynucleotide that encodes a polypeptide comprising the amino acid sequence of SEQ ID NO: 217.

In some embodiments, a polynucleotide encoding a centrosomal protein of 290 kDa polypeptide is a polynucleotide that encodes an N-terminal truncation, a C-terminal truncation, or a fragment of the amino acid sequence of SEQ ID NO: 217. N-terminal truncations, C-terminal truncations, or fragments may comprise at least 10, at least 12, at least 14, at least 16, at least 18, at least 20, at least 30, at least 40, at least 50, at least 75, at least 100, at least 200, at least 300, at least 400, at least 500, at least 600, at least 700, at least 800, at least 900, at least 1,000, but fewer than 2,479, consecutive amino acids of SEQ ID NO: 217.

In some embodiments, a polynucleotide of the present disclosure encodes a polypeptide. In some embodiments, the polypeptide is a human polypeptide. In some embodiments, the polynucleotide comprises the coding sequence of a wild-type gene (see e.g., SEQ ID NOs: 378-734), or a codon-optimized variant thereof (see e.g., SEQ ID NOs 735-738 and/or SEQ ID NOs: 741-743). In some embodiments, a polynucleotide encoding a polypeptide is a polynucleotide that encodes a polypeptide comprising an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an amino acid sequence selected from SEQ ID NOs: 21-377 and/or SEQ ID NOs: 739-740. In some embodiments, a polynucleotide of the present disclosure encodes a polypeptide comprising an amino acid sequence selected from SEQ ID NOs: 21-377 and/or SEQ ID NOs: 739-740.

In some embodiments, a polynucleotide encoding a polypeptide is a polynucleotide that encodes an N-terminal truncation, a C-terminal truncation, or a fragment of the amino acid sequence selected from SEQ ID NOs: 21-377 and/or SEQ ID NOs: 739-740. N-terminal truncations, C-terminal truncations, or fragments may comprise, for example, at least 10, at least 12, at least 14, at least 16, at least 18, at least 20, at least 30, at least 40, at least 50, at least 75, at least 100, at least 200, at least 300, at least 400, etc., consecutive amino acids selected from SEQ ID NOs: 21-377 and/or SEQ ID NOs: 739-740.

In some embodiments, a polynucleotide of the present disclosure encodes any one or more of a/an sterile alpha motif domain-containing protein 11, nephrocystin-4, espin, nicotinamide/nicotinic acid mononucleotide adenylyltransferase 1, mitofusin-2, ER membrane protein complex subunit 1, phospholipase A2 group V, dehydrodolichyl diphosphate synthase complex subunit, palmitoyl-protein thioesterase 1, elongation of very long chain fatty acids protein 1, Protein O-linked-mannose beta-1,2-N-acetylglucosaminyltransferase 1, Retinoid isomerohydrolase, Retinal-specific phospholipid-transporting ATPase, Collagen alpha-1 (XI), Guanine nucleotide-binding protein G(t) subunit alpha-2, Chloride channel CLIC-like protein 1, DNA damage-regulated autophagy modulator protein 2, U4/U6 small nuclear ribonucleoprotein Prp3, Alpha-endosulfine, Semaphorin-4A, Cyclic AMP-dependent transcription factor ATF-6 alpha, Hemicentin-1, Complement factor H, Protein crumbs homolog 1, Adiponectin receptor protein 1, Protein RD3, Serine/threonine-protein kinase Nek2, Feline leukemia virus subgroup C receptor-related protein 1, Usherin, Serologically defined colon cancer antigen 8, Olfactory receptor 2W3, NBAS subunit of NRZ tethering complex, Cytosolic carboxypeptidase-like protein 5, Zinc finger protein 513, Intraflagellar transport protein 172 homolog, Photoreceptor cilium actin regulator, EGF-containing fibulin-like extracellular matrix protein 1/TLE family member 5, Protein FAM161A, WD repeat-containing and planar cell polarity effector protein fritz homolog, Centrosome-associated protein ALMS1, U5 small nuclear ribonucleoprotein 200 kDa helicase, Metal transporter CNNM4, Cyclic nucleotide-gated cation channel alpha-3, Nephrocystin-1, Tyrosine-protein kinase Mer, Bardet-Biedl syndrome 5 protein, Ceramide kinase-like protein, Neurogenic differentiation factor 1, Transmembrane protein 237, Inward rectifier potassium channel 13, S-arrestin, Secreted phosphoprotein 24, CCA tRNA nucleotidyltransferase 1, mitochondrial, Sodium bicarbonate cotransporter 3, Leucine zipper transcription factor-like protein 1, Guanine nucleotide-binding protein G(t) subunit alpha-1, Three-prime repair exonuclease 1, MAP kinase-activated protein kinase 3, Ataxin-7, Vitamin K-dependent protein S, ADP-ribosylation factor-like protein 6, Interphotoreceptor matrix proteoglycan 2, IQ calmodulin-binding motif-containing protein 1, Rhodopsin, Nephrocystin-3, Clarin-1, Probable cationic amino acid transporter/Solute carrier family 7 member 14, Choline-phosphate cytidylyltransferase A, Centrosomal protein of 19 kDa, Rod cGMP-specific 3′,5′-cyclic phosphodiesterase subunit beta, Wolframin, Homeobox protein HMX1, Ras-related protein Rab-28, Coiled-coil and C2 domain-containing protein 2A, Prominin-1, Adhesion G protein-coupled receptor A3, Death domain-containing protein 1, WD repeat-containing protein 19, cGMP-gated cation channel alpha-1, CDGSH iron-sulfur domain-containing protein 2, Microsomal triglyceride transfer protein large subunit, Leucine-rich repeat, immunoglobulin-like domain and transmembrane domain-containing protein 3, Bardet-Biedl syndrome 7 protein, Bardet-Biedl syndrome 12 protein, Major facilitator superfamily domain-containing protein 8, Serine/threonine-protein kinase PLK4, Lecithin retinol acyltransferase, Toll-like receptor 3, Cytochrome P450 4V2, Spliceosome-associated protein CWC27 homolog, Centrosomal protein POC5, Versican core protein, Adhesion G-protein coupled receptor V1, COUP transcription factor 1, Mitochondrial outer membrane protein SLC25A46, Catenin alpha-1, Histidine-tRNA ligase, cytoplasmic, Rod cGMP-specific 3′,5′-cyclic phosphodiesterase subunit alpha, Metabotropic glutamate receptor 6, Serine/threonine-protein kinase MAK, Complement C2, Complement factor B, Tubby-related protein 1, Guanylyl cyclase-activating protein 1, Guanylyl cyclase-activating protein 2, Peripherin-2, Interphotoreceptor matrix proteoglycan 1, Protein eyes shut homolog, Collagen alpha-1 (IX) chain, Regulating synaptic membrane exocytosis protein 1, Lebercilin, Elongation of very long chain fatty acids protein 4, PR domain zinc finger protein 13, Reticulon-4-interacting protein 1, mitochondrial, Jouberin, Peroxisomal targeting signal 2 receptor, CCR4-NOT transcription complex subunit 9, Aryl hydrocarbon receptor, Kelch-like protein 7, Retinitis pigmentosa 9 protein, Protein PTHB1, Peroxisomal ATPase PEX1, Tetraspanin-12, Inosine-5′-monophosphate dehydrogenase 1, Short-wave-sensitive opsin 1, UPF0606 protein KIAA1549, Retinitis pigmentosa 1-like 1 protein, Disintegrin and metalloproteinase domain-containing protein 9, Heparan-alpha-glucosaminide N-acetyltransferase, Oxygen-regulated protein 1, Alpha-tocopherol transfer protein, Centrosome and spindle pole-associated protein 1, Dynamin-like 120 kDa protein, mitochondrial, Peroxisome biogenesis factor 2, Cyclic nucleotide-gated cation channel beta-3, Cilia- and flagella-associated protein 418, Growth/differentiation factor 6, Regulating synaptic membrane exocytosis protein 2, Potassium voltage-gated channel subfamily V member 2, E3 ubiquitin-protein ligase Topors, Centrosomal protein of 78 kDa, Inversin, U4/U6 small nuclear ribonucleoprotein Prp4, Whirlin, E3 ubiquitin-protein ligase TRIM32, Toll-like receptor 4, Cytoplasmic dynein 2 intermediate chain 2, Programmed cell death protein 2, Exosome complex component RRP4, Phosphatidylinositol polyphosphate 5-phosphatase type IV, Phytanoyl-CoA dioxygenase, peroxisomal, Acyl-CoA-binding domain-containing protein, Protocadherin-15, Retinol-binding protein 3, DNA excision repair protein ERCC-6, Hexokinase-1, Cadherin-23, Cadherin-related family member 1, RPE-retinal G protein-coupled receptor, Kinesin-like protein KIF11, Retinol-binding protein 4, Cone cGMP-specific 3′,5′-cyclic phosphodiesterase subunit alpha, Paired box protein Pax-2, PDZ domain-containing protein 7, ADP-ribosylation factor-like protein 3, BBSome-interacting protein 1, Age-related maculopathy susceptibility protein 2, Serine protease HTRA1, Ornithine aminotransferase, mitochondrial, Zinc finger protein 408, Tubby protein homolog, Transcriptional enhancer factor TEF-1, Harmonin, Transmembrane protein 216, Bestrophin-1, Isoaspartyl peptidase/L-asparaginase, Rod outer segment membrane protein 1, Bardet-Biedl syndrome 1 protein, Calcium-binding protein 4, Low-density lipoprotein receptor-related protein 5, Calpain-5, Unconventional myosin-VIIa, Transmembrane protein 126A, Frizzled-4, Cytoplasmic dynein 2 light intermediate chain 1, Centrosomal protein of 164 kDa, Complement C1q tumor necrosis factor-related protein 5, Membrane frizzled-related protein, Voltage-dependent calcium channel subunit alpha-2/delta-4, Guanine nucleotide-binding protein G(I)/G(S)/G(T) subunit beta-3, Retinal cone rhodopsin-sensitive cGMP 3′,5′-cyclic phosphodiesterase subunit gamma, Collagen alpha-1 (II) chain, Matrix metalloproteinase-19, Retinol dehydrogenase 5, T-complex protein 1 subunit beta, Bardet-Biedl syndrome 10 protein, Centrosomal protein of 290 kDa, POC1 centriolar protein homolog B, Mevalonate kinase, Intraflagellar transport protein 81 homolog, Mitochondrial translation release factor in rescue, Integral membrane protein 2B, Retinoblastoma-associated protein, RCC1 and BTB domain-containing protein 1, Rhodopsin kinase GRK1, X-linked retinitis pigmentosa GTPase regulator-interacting protein 1, Neural retina-specific leucine zipper protein, Homeobox protein OTX2, Retinol dehydrogenase 11, Retinol dehydrogenase 12, Tubulin polyglutamylase TTLL5, Spermatogenesis-associated protein 7, Tetratricopeptide repeat protein 8, Fibulin-5, Transient receptor potential cation channel subfamily M member 1, Gamma-tubulin complex component 4, Sodium/potassium/calcium exchanger 1, Photoreceptor-specific nuclear receptor, Bardet-Biedl syndrome 4 protein, Calcium and integrin-binding family member 2, Retinaldehyde-binding protein 1, N-acetylglucosamine-1-phosphotransferase subunit gamma, Intraflagellar transport protein 140 homolog, Clusterin-associated protein 1, ATP-binding cassette sub-family C member 6, Ketimine reductase mu-crystallin, Battenin, Zinc finger protein 423, Protein fantom, Bardet-Biedl syndrome 2 protein, ADP-ribosylation factor-like protein 2-binding protein, Cyclic nucleotide-gated cation channel beta-1, Cadherin-3, Pre-mRNA-splicing factor ATP-dependent RNA helicase PRP16, A disintegrin and metalloproteinase with thrombospondin motifs 18, Solute carrier family 38 member 8, Retinal guanylyl cyclase 1, Pre-mRNA-processing-splicing factor 8, Aryl-hydrocarbon-interacting protein-like 1, Membrane-associated phosphatidylinositol transfer protein 3, Protein unc-119 homolog A, Probable G-protein coupled receptor 179, Tectonic-like complex member MKS1, Carbonic anhydrase 4, Regulator of G-protein signaling 9, Arylsulfatase G, pre-mRNA splicing regulator USH1G, Photoreceptor disk component PRCD, Fascin-2, Retinal rod rhodopsin-sensitive cGMP 3′,5′-cyclic phosphodiesterase subunit gamma, Laminin subunit alpha-1, AFG3-like protein 2, Cone-rod homeobox protein, Receptor expression-enhancing protein 6, Retina and anterior neural fold homeobox protein 2, Complement C3, Rho guanine nucleotide exchange factor 18, Patatin-like phospholipase domain-containing protein 6, Regulator of G-protein signaling 9-binding protein, Optic atrophy 3 protein, U4/U6 small nuclear ribonucleoprotein Prp31, Isocitrate dehydrogenase [NAD] subunit beta, mitochondrial, Pantothenate kinase 2, mitochondrial, Protein jagged-1, Molecular chaperone MKKS, Centrosomal protein kizuna, Lysophosphatidylserine lipase ABHD12, Kinesin-like protein KIF3B, Centrosome-associated protein CEP250, Pre-mRNA-processing factor 6, Cilia- and flagella-associated protein 410, Dynamin-1-like protein, Metalloproteinase inhibitor 3, Intraflagellar transport protein 27 homolog, Fibulin-1, MIEF1 upstream open reading frame protein, Aconitate hydratase, mitochondrial, Gamma-tubulin complex component 6, Centriole and centriolar satellite protein, Retinoschisin, Protein XRP2, Dystrophin, X-linked retinitis pigmentosa GTPase regulator, Nyctalopin, Xaa-Pro dipeptidase, Norrin, Voltage-dependent L-type calcium channel subunit alpha-1F, Phosphoglycerate kinase 1, Rab proteins geranylgeranyltransferase component A 1, Mitochondrial import inner membrane translocase subunit Tim8 A, Ribose-phosphate pyrophosphokinase 1, Long-wave-sensitive opsin 1, Medium-wave-sensitive opsin 1, Short-wave-sensitive opsin 1, Transcription factor A, mitochondrial, NADH-ubiquinone oxidoreductase chain 1, NADH-ubiquinone oxidoreductase chain 2, NADH-ubiquinone oxidoreductase chain 3, NADH-ubiquinone oxidoreductase chain 4L, NADH-ubiquinone oxidoreductase chain 4, NADH-ubiquinone oxidoreductase chain 5, NADH-ubiquinone oxidoreductase chain 6, ATP synthase subunit a, ATP synthase protein 8, Cytochrome c oxidase subunit 1, Cytochrome c oxidase subunit 3, Cytochrome b, Leucine-tRNA ligase, mitochondrial, Nondiscriminating glutamyl-tRNA synthetase EARS2, mitochondrial, Lysine-tRNA ligase, Histidine-tRNA ligase, mitochondrial, Serine-tRNA ligase, mitochondrial, Probable proline-tRNA ligase, mitochondrial, Cyanocobalamin reductase/alkylcobalamin dealkylase, POU domain, class 3, transcription factor 4, Ribosomal protein S6 kinase alpha-6, Ciliogenesis and planar polarity effector 1, Meckelin, TRAF3-interacting protein 1, Intraflagellar transport protein 74 homolog, S phase cyclin A-associated protein in the endoplasmic reticulum, Sodium channel and clathrin linker 1, Protein TALPID3, Tectonic-2, ADP-ribosylation factor-like protein 13B, B9 domain-containing protein 1, B9 domain-containing protein 2, C2 domain-containing protein 3, Centrosomal protein of 41 kDa, Centrosomal protein of 104 kDa, Centrosomal protein of 120 kDa, Intraflagellar transport protein 172 homolog, Katanin-interacting protein, Kinesin-like protein KIF7, Retinal rod rhodopsin-sensitive cGMP 3′,5′-cyclic phosphodiesterase subunit delta, Tectonic-1, Tectonic-3, Transmembrane protein 107, Transmembrane protein 138, Transmembrane protein 231, Tetratricopeptide repeat protein 21B, Nuclear receptor ROR-alpha, Beta-nerve growth factor, Collagen alpha-2 (VIII) chain, Solute carrier family 4 member 11, Zinc finger E-box-binding homeobox 1, Keratin, type II cuticular Hb3, Keratin, type I cytoskeletal 12, Transforming growth factor-beta-induced protein ig-h3, Tumor-associated calcium signal transducer 2, Carbohydrate sulfotransferase 6, Gelsolin, UbiA prenyltransferase domain-containing protein 1, Decorin, 1-phosphatidylinositol 3-phosphate 5-kinase, Transcription factor Ovo-like 2, Grainyhead-like protein 2 homolog, and/or any chimeric polypeptides thereof, in any suitable combination.

A polynucleotide of the present disclosure encoding a polypeptide may further encode additional coding and non-coding sequences. Examples of additional coding and non-coding sequences may include, but are not limited to, sequences encoding additional polypeptide tags (e.g., encoded in-frame with the polypeptide in order to produce a fusion protein), introns (e.g., native, modified, or heterologous introns), 5′ and/or 3′ UTRs (e.g., native, modified, or heterologous 5′ and/or 3′ UTRs), and the like. Examples of suitable polypeptide tags may include, but are not limited, to any combination of purification tags, such as his-tags, flag-tags, maltose binding protein and glutathione-S-transferase tags, detection tags, such as tags that may be detected photometrically (e.g., green fluorescent protein, red fluorescent protein, etc.) and tags that have a detectable enzymatic activity (e.g., alkaline phosphatase, etc.), tags containing secretory sequences, signal sequences, leader sequences, and/or stabilizing sequences, protease cleavage sites (e.g., furin cleavage sites, TEV cleavage sites, Thrombin cleavage sites, etc.), and the like. In some embodiments, the 5′ and/or 3′UTRs increase the stability, localization, and/or translational efficiency of the polynucleotides. In some embodiments, the 5′ and/or 3′UTRs improve the level and/or duration of protein expression. In some embodiments, the 5′ and/or 3′UTRs include elements (e.g., one or more miRNA binding sites, etc.) that may block or reduce off-target expression (e.g., inhibiting expression in specific cell types (e.g., neuronal cells), at specific times in the cell cycle, at specific developmental stages, etc.). In some embodiments, the 5′ and/or 3′UTRs include elements (e.g., one or more miRNA binding sites, etc.) that may enhance expression of the encoded polypeptide in specific cell types.

In some embodiments, a polynucleotide of the present disclosure encoding a polypeptide is operably linked to one or more (e.g., one or more, two or more, three or more, four or more, five or more, ten or more, etc.) regulatory sequences. The term “regulatory sequence” may include enhancers, insulators, promoters, and other expression control elements (e.g., polyadenylation signals). Any suitable enhancer(s) known in the art may be used, including, for example, enhancer sequences from mammalian genes (such as globin, elastase, albumin, α-fetoprotein, insulin and the like), enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like), and any combinations thereof. Any suitable insulator(s) known in the art may be used, including, for example, HSV chromatin boundary (CTRL/CTCF-binding/insulator) elements CTRL1 and/or CTRL2, chicken hypersensitive site 4 insulator (cHS4), human HNRPA2B1-CBX3 ubiquitous chromatin opening element (UCOE), the scaffold/matrix attachment region (S/MAR) from the human interferon beta gene (IFNB1), and any combinations thereof. Any suitable promoter (e.g., suitable for transcription in mammalian host cells) known in the art may be used, including, for example, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), promoters from heterologous mammalian genes (such as the actin promoter (e.g., the β-actin promoter), a ubiquitin promoter (e.g., a ubiquitin C (UbC) promoter), a phosphoglycerate kinase (PGK) promoter, an immunoglobulin promoter, from heat-shock promoters, and the like), promoters from homologous mammalian genes, synthetic promoters (such as the CAG promoter), and any combinations thereof, provided such promoters are compatible with the host cells. Regulatory sequences may include those which direct constitutive expression of a nucleic acid, as well as tissue-specific regulatory and/or inducible or repressible sequences.

In some embodiments, a polynucleotide of the present disclosure is operably linked to one or more heterologous promoters. In some embodiments, the one or more heterologous promoters are one or more of constitutive promoters, tissue-specific promoters, temporal promoters, spatial promoters, inducible promoters, and repressible promoters. In some embodiments, the one or more heterologous promoters are one or more of the human cytomegalovirus (HCMV) immediate early promoter, the human elongation factor-1 (EF1) promoter, the human β-actin promoter, the human UbC promoter, the human PGK promoter, the synthetic CAGG promoter, and any combinations thereof. In some embodiments, a polynucleotide of the present disclosure encoding a polypeptide is operably linked to an HCMV promoter.

In some embodiments, a polynucleotide of the present disclosure encoding a polypeptide expresses the polypeptide when the polynucleotide is delivered into one or more target cells of a subject (e.g., one or more cells of the eye, anterior chamber, posterior chamber, ciliary body, corneal epithelial cell, rod cell, cone cell, photoreceptor, retinal pigment epithelial cell, retinal ganglion cell, bipolar cell, horizontal cell, muller cell, amacrine cell, etc. of the subject). In some embodiments, expression of the polypeptide enhances, increases, augments, and/or supplements the levels, function, and/or activity of the polypeptide in one or more target cells of a subject (e.g., as compared to prior to expression of the polypeptide, as compared to levels of the endogenous polypeptide expressed in the cell, etc.). In some embodiments, expression of the polypeptide provides prophylactic, palliative, or therapeutic relief of one or more signs or symptoms of an eye condition or disease in a subject (e.g., as compared to prior to expression of the polypeptide).

### Chimeric Polypeptides

In some embodiments, a polynucleotide of the present disclosure encodes a chimeric polypeptide comprising a first polypeptide and a second polypeptide. In some embodiments, the first and second polypeptides are the same. In some embodiments, the first and second polypeptides are different. In some embodiments, the chimeric polypeptide further comprises a linker polypeptide linking the first and second polypeptides. In some embodiments, the chimeric polypeptide comprises, from n-terminus to c-terminus, the first polypeptide-the linker polypeptide-the second polypeptide. The first and/or second polypeptides may be any of the polypeptides described herein or known in the art.

In some embodiments, the linker polypeptide is a cleavable linker polypeptide. Any cleavable linker polypeptide known in the art may be used in the chimeric polypeptides of the present disclosure, including, for example, a T2A linker, a P2A linker, a E2A linker, and F2A linker, etc. In some embodiments, the linker polypeptide is a T2A linker polypeptide. An exemplary nucleic acid sequence encoding a T2A linker polypeptide is provided as SEQ ID NO: 1. An exemplary amino acid sequence of a T2A linker polypeptide is provided as SEQ ID NO: 5. In some embodiments, the linker polypeptide is a P2A linker polypeptide. An exemplary nucleic acid sequence encoding a P2A linker polypeptide is provided as SEQ ID NO: 2. An exemplary amino acid sequence of a P2A linker polypeptide is provided as SEQ ID NO: 6. In some embodiments, the linker polypeptide is an E2A linker polypeptide. An exemplary nucleic acid sequence encoding an E2A linker polypeptide is provided as SEQ ID NO: 3. An exemplary amino acid sequence of an E2A linker polypeptide is provided as SEQ ID NO: 7. In some embodiments, the linker polypeptide is an F2A linker polypeptide. An exemplary nucleic acid sequence encoding an F2A linker polypeptide is provided as SEQ ID NO: 4. An exemplary amino acid sequence of an F2A linker polypeptide is provided as SEQ ID NO: 8. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 68-71. In some embodiments, the linker polypeptide comprises a sequence selected from SEQ ID NOS: 5-8.

In some embodiments, the linker polypeptide is a non-cleavable linker polypeptide. Any non-cleavable linker polypeptide known in the art may be used in the chimeric polypeptides of the present disclosure, including, for example, a GGGGSGGGGSGGGGS (SEQ ID NO: 9) linker, a GGSSRSSSSGGGGSGGGG (SEQ ID NO: 10) linker, a GGGGSGGGGSGGGGGGGGS (SEQ ID NO: 11) linker, a CGGGSGGGGSGGGGS (SEQ ID NO: 12) linker, a SHGGHGGGGSGGGGS (SEQ ID NO: 13) linker, a MGGMSGGGGSGGGGS (SEQ ID NO: 14) linker, a YGGYSGGGGSGGGGS (SEQ ID NO: 15) linker, a WGGYSGGGGSGGGGS (SEQ ID NO: 16) linker, a SVSVGMKPSPRP (SEQ ID NO: 17) linker, a VISNHAGSSRRL (SEQ ID NO: 18) linker, a PWIPTPRPTFTG (SEQ ID NO: 19) linker, a RGRGRGRGRGR (SEQ ID NO: 20) linker, etc. In some embodiments, the linker polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to an amino acid sequence selected from SEQ ID NOS: 9-20. In some embodiments, the linker polypeptide comprises a sequence selected from SEQ ID NOS: 9-20.

In some embodiments, a polynucleotide of the present disclosure does not comprise the coding sequence of (e.g., a transgene encoding) a Collagen alpha-1 (VII) chain polypeptide (COL7). In some embodiments, a polynucleotide of the present disclosure does not comprise the coding sequence of (e.g., a transgene encoding) a Lysyl hydroxylase 3 polypeptide (LH3). In some embodiments, a polynucleotide of the present disclosure does not comprise the coding sequence of (e.g., a transgene encoding) a Keratin type I cytoskeletal 17 polypeptide (KRT17). In some embodiments, a polynucleotide of the present disclosure does not comprise the coding sequence of (e.g., a transgene encoding) a transglutaminase (TGM) polypeptide (e.g., a human transglutaminase polypeptide such as a human TGM1 polypeptide and/or a human TGM5 polypeptide). In some embodiments, a polynucleotide of the present disclosure does not comprise the coding sequence of (e.g., a transgene encoding) a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a polynucleotide of the present disclosure does not comprise the coding sequence of (e.g., a transgene encoding) an antibody (e.g., a full-length antibody, an antibody fragment, etc.). In some embodiments, a polynucleotide of the present disclosure does not comprise the coding sequence of (e.g., a transgene encoding) a Serine Protease Inhibitor Kazal-type (SPINK) polypeptide (e.g., a human SPINK polypeptide, such as a SPINK5 polypeptide). In some embodiments, a polynucleotide of the present disclosure does not comprise the coding sequence of (e.g., a transgene encoding) a filaggrin or filaggrin 2 polypeptide (e.g., a human filaggrin or filaggrin 2 polypeptide). In some embodiments, a polynucleotide of the present disclosure does not comprise the coding sequence of (e.g., a transgene encoding) a Cystic Fibrosis Transmembrane Conductance Regulator (CFTR) polypeptide (e.g., a human CFTR polypeptide). In some embodiments, a polynucleotide of the present disclosure does not comprise the coding sequence of (e.g., a transgene encoding) an ichthyosis-associated polypeptide (e.g., an ATP-binding cassette sub-family A member 12 polypeptide, a 1-acylglycerol-3-phosphate O-acyltransferase ABHD5 polypeptide, an Aldehyde dehydrogenase family 3 member A2 polypeptide, an Arachidonate 12-lipoxygenase 12R-type polypeptide, a Hydroperoxide isomerase ALOXE3 polypeptide, an AP-1 complex subunit sigma-1A polypeptide, an Arylsulfatase E polypeptide, a Caspase-14 polypeptide, a Corneodesmosin polypeptide, a Ceramide synthase 3 polypeptide, a Carbohydrate sulfotransferase 8 polypeptide, a Claudin-1 polypeptide, a Cystatin-A polypeptide, a Cytochrome P450 4F22 polypeptide, a 3-beta-hydroxysteroid-Delta (8), Delta (7)-isomerase polypeptide, an Elongation of very long chain fatty acids protein 4 polypeptide, a Filaggrin polypeptide, a Filaggrin 2 polypeptide, a Gap junction beta-2 polypeptide, a Gap junction beta-3 polypeptide, a Gap junction beta-4 polypeptide, a Gap junction beta-6 polypeptide, a 3-ketodihydrosphingosine reductase polypeptide, a Keratin, type II cytoskeletal 1 polypeptide, a Keratin, type II cytoskeletal 2 epidermal polypeptide, a Keratin, type I cytoskeletal 9 polypeptide, a Keratin, type I cytoskeletal 10 polypeptide, a Lipase member N polypeptide, a Loricrin polypeptide, a Membrane-bound transcription factor site-2 protease polypeptide, a Magnesium transporter NIPA4 polypeptide, a Sterol-4-alpha-carboxylate 3-dehydrogenase, decarboxylating polypeptide, a Peroxisomal targeting signal 2 receptor polypeptide, a D-3-phosphoglycerate dehydrogenase polypeptide, a Phytanoyl-CoA dioxygenase, peroxisomal polypeptide, Patatin-like phospholipase domain-containing protein 1 polypeptide, a Proteasome maturation protein polypeptide, a Phosphoserine aminotransferase polypeptide, a Short-chain dehydrogenase/reductase family 9C member 7 polypeptide, a Serpin B8 polypeptide, a Long-chain fatty acid transport protein 4 polypeptide, a Synaptosomal-associated protein 29 polypeptide, a Suppressor of tumorigenicity 14 protein polypeptide, a Steryl-sulfatase polypeptide, a Vacuolar protein sorting-associated protein 33B polypeptide, and a CAAX prenyl protease 1 homolog polypeptide). In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a polynucleotide of the present disclosure does not comprise the coding sequence of (e.g., a transgene encoding) a Collagen alpha-1 (VII) chain polypeptide, a Lysyl hydroxylase 3 polypeptide, a Keratin type I cytoskeletal 17 polypeptide, a transglutaminase (TGM) polypeptide, a filaggrin polypeptide, a cosmetic protein, an antibody, a SPINK polypeptide, a CFTR polypeptide, an ichthyosis-associated polypeptide, an Alpha-1-antitrypsin polypeptide, a Sodium-dependent phosphate transport protein 2B polypeptide, a Dynein heavy chain 5 axonemal polypeptide, a Dynein heavy chain 11 axonemal polypeptide, a Coiled-coil domain-containing protein 39 polypeptide, a Dynein intermediate chain 1 axonemal polypeptide, a Coiled-coil domain-containing protein 40 polypeptide, a Coiled-coil domain containing protein 103 polypeptide, a Sperm-associated antigen 1 polypeptide, a Zinc finger MYND domain-containing protein 10 polypeptide, an Armadillo repeat containing protein 4 polypeptide, a Coiled-coil domain-containing protein 151 polypeptide, a Dynein intermediate chain 2 axonemal polypeptide, a Radial spoke head 1 homolog polypeptide, a Coiled-coil domain-containing protein 114 polypeptide, a Radial spoke head protein 4 homolog A polypeptide, a Dynein assembly factor 1 axonemal polypeptide, a Dynein assembly factor 2 axonemal polypeptide, a Leucine-rich repeat-containing protein 6 polypeptide, a Pulmonary surfactant-associated protein B polypeptide, a Pulmonary surfactant-associated protein C polypeptide, a Homeobox protein Nkx-2.1 polypeptide, an ATP-binding cassette sub-family A member 3 polypeptide, a Cytokine receptor common subunit beta polypeptide, a Granulocyte-macrophage colony-stimulating factor receptor subunit alpha polypeptide, a Bone morphogenetic protein receptor type-2 polypeptide, a Sarcoplasmic/endoplasmic reticulum calcium ATPase 2 polypeptide, a serine/threonine-protein kinase receptor R3 polypeptide, an Endoglin polypeptide, a Mothers against decapentaplegic homolog 9 polypeptide, a Caveolin-1 polypeptide, a Potassium channel subfamily K member 3 polypeptide, an elF-2-alpha kinase GCN2 polypeptide, a Pulmonary surfactant-associated protein A2 polypeptide, a Telomerase reverse transcriptase polypeptide, a Dyskerin polypeptide, a Regulator of telomere elongation helicase 1 polypeptide, a Poly(A)-specific ribonuclease PARN polypeptide, a TERF1-interacting nuclear factor 2 polypeptide, an H/ACA ribonucleoprotein complex non-core subunit NAF1 polypeptide, a Mucin-5B polypeptide, a Desmoplakin polypeptide, a CST complex subunit STN1 polypeptide, a Dipeptidyl peptidase 9 polypeptide, and/or any chimeric polypeptides thereof.

In some embodiments, a polynucleotide of the present disclosure does not comprise the coding sequence of (e.g., a transgene encoding) an immunomodulatory polypeptide. In some embodiments, a polynucleotide of the present disclosure does not comprise the coding sequence of (e.g., a transgene encoding) a cytokine polypeptide and/or a chemokine polypeptide. In some embodiments, a polynucleotide of the present disclosure does not comprise the coding sequence of (e.g., a transgene encoding) a cytokine polypeptide. In some embodiments, a polynucleotide of the present disclosure does not comprise the coding sequence of (e.g., a transgene encoding) an IL-1 polypeptide, an IL-2 polypeptide, an IL-7 polypeptide, an IL-12 polypeptide, an IL-13 polypeptide, an IL-15 polypeptide, an IL-17 polypeptide, an IL-18 polypeptide, an IL-28 polypeptide, an IL-32 polypeptide, an IL-33 polypeptide, an IL-34 polypeptide, a TNFα polypeptide, an IFNγ polypeptide, a G-CSF polypeptide, a GM-CSF polypeptide, and/or any chimeric polypeptides thereof.

In some embodiments, a polynucleotide of the present disclosure does not comprise the coding sequence of (e.g., a transgene encoding) a chemokine polypeptide. In some embodiments, a polynucleotide of the present disclosure does not comprise the coding sequence of (e.g., a transgene encoding) a CXCL1 polypeptide, a CXCL2 polypeptide, a CXCL8 polypeptide, a CXCL9 polypeptide, a CXCL11 polypeptide, a CXCL16 polypeptide, a CCL2 polypeptide, a CCL3 polypeptide, a CCL4 polypeptide, a CCL5 polypeptide, a CCL11 polypeptide, and/or any chimeric polypeptides thereof.

### Recombinant Nucleic Acids

In some embodiments, the present disclosure relates to recombinant nucleic acids comprising any one or more of the polynucleotides described herein. In some embodiments, the recombinant nucleic acid is a vector (e.g., an expression vector, a display vector, etc.). In some embodiments, the vector is a DNA vector or an RNA vector. Generally, vectors suitable to maintain, propagate, and/or express polynucleotides to produce one or more polypeptides in a subject may be used. Examples of suitable vectors may include, for example, plasmids, cosmids, episomes, transposons, and viral vectors (e.g., adenoviral vectors, adeno-associated viral vectors, vaccinia viral vectors, Sindbis-viral vectors, measles vectors, herpes viral vectors, lentiviral vectors, retroviral vectors, etc.). In some embodiments, the vector is a herpes viral vector. In some embodiments, the vector is capable of autonomous replication in a host cell. In some embodiments, the vector is incapable of autonomous replication in a host cell. In some embodiments, the vector can integrate into a host DNA. In some embodiments, the vector cannot integrate into a host DNA (e.g., is episomal). Methods of making vectors containing one or more polynucleotides of interest are well known to one of ordinary skill in the art, including, for example, by chemical synthesis or by artificial manipulation of isolated segments of nucleic acids (e.g., by genetic engineering techniques).

In some embodiments, a recombinant nucleic acid of the present disclosure is a herpes simplex virus (HSV) amplicon. Herpes virus amplicons, including the structural features and methods of making the same, are generally known to one of ordinary skill in the art (see e.g., de Silva S. and Bowers W. “Herpes Virus Amplicon Vectors”. Viruses 2009, 1, 594-629). In some embodiments, the herpes simplex virus amplicon is an HSV-1 amplicon. In some embodiments, the herpes simplex virus amplicon is an HSV-1 hybrid amplicon. Examples of HSV-1 hybrid amplicons may include, but are not limited to, HSV/AAV hybrid amplicons, HSV/EBV hybrid amplicons, HSV/EBV/RV hybrid amplicons, and/or HSV/Sleeping Beauty hybrid amplicons. In some embodiments, the amplicon is an HSV/AAV hybrid amplicon. In some embodiments, the amplicon is an HSV/Sleeping Beauty hybrid amplicon.

In some embodiments, a recombinant nucleic acid of the present disclosure is a recombinant herpes virus genome. The recombinant herpes virus genome may be a recombinant genome from any member of the Herpesviridae family of DNA viruses known in the art, including, for example, a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any combinations or derivatives thereof. In some embodiments, the recombinant herpes virus genome comprises one or more (e.g., one or more, two or more, three or more, four or more, five or more, six or more, seven or more, eight or more, nine or more, ten or more, etc.) inactivating mutations. As used herein, an “inactivating mutation” may refer to any mutation that results in a gene or regulon product (RNA or protein) having reduced, undetectable, or eliminated quantity and/or function (e.g., as compared to a corresponding sequence lacking the inactivating mutation). Examples of inactivating mutations may include, but are not limited to, deletions, insertions, point mutations, and rearrangements in transcriptional control sequences (promoters, enhancers, insulators, etc.) and/or coding sequences of a given gene or regulon. Any suitable method of measuring the quantity of a gene or regulon product known in the art may be used, including, for example, qPCR, Northern blots, RNAseq, western blots, ELISAs, etc. In some embodiments, the one or more inactivating mutations are in one or more (e.g., one or more, two or more, three or more, four or more, five or more, six or more, seven or more, eight or more, nine or more, ten or more, etc.) herpes virus genes. In some embodiments, the recombinant herpes virus genome is attenuated (e.g., as compared to a corresponding, wild-type herpes virus genome). In some embodiments, the recombinant herpes virus genome is replication competent. In some embodiments, the recombinant herpes virus genome is replication defective. In some embodiments, the recombinant herpes virus genome is not oncolytic.

In some embodiments, the recombinant nucleic acid is a recombinant herpes simplex virus (HSV) genome. In some embodiments, the recombinant herpes simplex virus genome comprises one or more (e.g., one or more, two or more, three or more, four or more, five or more, six or more, seven or more, eight or more, nine or more, ten or more, etc.) inactivating mutations. In some embodiments, the one or more inactivating mutations are in one or more (e.g., one or more, two or more, three or more, four or more, five or more, six or more, seven or more, eight or more, nine or more, ten or more, etc.) herpes simplex virus genes. In some embodiments, the recombinant herpes simplex virus genome is attenuated (e.g., as compared to a corresponding, wild-type herpes simplex virus genome). In some embodiments, the recombinant herpes simplex virus genome is replication competent. In some embodiments, the recombinant herpes simplex virus genome is replication defective. In some embodiments, the recombinant herpes simplex virus genome is not oncolytic.

In some embodiments, the recombinant herpes virus genome is a recombinant herpes simplex virus type 1 (HSV-1) genome, a recombinant herpes simplex virus type 2 (HSV-2) genome, or any derivatives thereof. In some embodiments, the recombinant herpes simplex virus genome is a recombinant HSV-1 genome. In some embodiments, the recombinant HSV-1 genome may be from any HSV-1 strain known in the art, including, for example, strains 17, Ty25, R62, S25, Ku86, S23, R11, Ty148, Ku47, H166_{syn}, 1319-2005, F-13, M-12, 90237, F-17, KOS, 3083-2008, F12g, L2, CD38, H193, M-15, India 2011, 0116209, F-111, 66-207, 2762, 369-2007, 3355, Macintyre, McKrae, 7862, 7-hse, HF10, 1394,2005, 270-2007, OD4, SC16, M-19, 4J1037, 5J1060, J1060, KOS79, 132-1988, 160-1982, H166, 2158-2007, RE, 78326, F18g, F11, 172-2010, H129, F, E4, CJ994, F14g, E03, E22, E10, E06, E11, E25, E23, E35, E15, E07, E12, E14, E08, E19, E13, ATCC 2011, etc. (see e.g., Bowen et al. J Virol. 2019 Apr. 3; 93 (8)). In some embodiments, the recombinant HSV-1 genome is from the KOS strain. In some embodiments, the recombinant HSV-1 genome is not from the McKrae strain. In some embodiments, the recombinant HSV-1 genome is attenuated (e.g., as compared to a corresponding, wild-type HSV-1 genome). In some embodiments, the recombinant HSV-1 genome is replication competent. In some embodiments, the recombinant HSV-1 genome is replication defective. In some embodiments, the recombinant HSV-1 genome is not oncolytic.

In some embodiments, the recombinant herpes simplex virus genome comprises an inactivating mutation in at least one, at least two, at least three, at least four, at least five, at least six, at least seven, or all eight of the Infected Cell Protein (or Infected Cell Polypeptide) (ICP) 0, ICP4, ICP22, ICP27, ICP47, thymidine kinase (tk), Long Unique Region (UL) 41 and/or UL55 herpes simplex virus genes. In some embodiments, the recombinant herpes simplex virus genome does not comprise an inactivating mutation in the ICP34.5 (one or both copies) and/or ICP47 herpes simplex virus genes (e.g., to avoid production of an immune-stimulating virus). In some embodiments, the recombinant herpes simplex virus genome does not comprise an inactivating mutation in the ICP34.5 (one or both copies) herpes simplex virus gene. In some embodiments, the recombinant herpes simplex virus genome does not comprise an inactivating mutation in the ICP47 herpes simplex virus gene. In some embodiments, the recombinant herpes simplex virus genome does not comprise an inactivating mutation in the ICP34.5 (one or both copies) and ICP47 herpes simplex virus genes. In some embodiments, the recombinant herpes simplex virus genome is not oncolytic. In some embodiments, the recombinant herpes simplex virus genome is not conditionally replication competent. In some embodiments, the recombinant herpes simplex virus genome is not conditionally replication competent in a cancerous cell.

In some embodiments, the recombinant herpes simplex virus genome comprises an inactivating mutation in the ICP0 gene (one or both copies). In some embodiments, the recombinant herpes simplex virus genome comprises an inactivating mutation in the ICP0 gene (one or both copies), and further comprises an inactivating mutation in the ICP4 (one or both copies), ICP22, ICP27, ICP47, UL41, and/or UL55 genes. In some embodiments, the recombinant herpes simplex virus genome comprises an inactivating mutation in the ICP0 gene (one or both copies), and an inactivating mutation in the ICP4 gene (one or both copies). In some embodiments, the recombinant herpes simplex virus genome comprises an inactivating mutation in the ICP0 gene (one or both copies), and an inactivating mutation in the ICP22 gene. In some embodiments, the recombinant herpes simplex virus genome comprises an inactivating mutation in the ICP0 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), and an inactivating mutation in the ICP22 gene. In some embodiments, the recombinant herpes simplex virus genome comprises an inactivating mutation in the ICP0 gene (one or both copies), an inactivating mutation in the ICP4 gene (one or both copies), and an inactivating mutation in the UL41 gene. In some embodiments, the recombinant herpes simplex virus genome comprises an inactivating mutation in the ICP0 gene (one or both copies), an inactivating mutation in the ICP22 gene, and an inactivating mutation in the UL41 gene. In some embodiments, the recombinant herpes simplex virus genome comprises an inactivating mutation in the ICP0 gene (one or both copies), an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP0 (one or both copies), ICP4 (one or both copies), ICP22, and/or UL41 genes. In some embodiments, the recombinant herpes simplex virus genome further comprises an inactivating mutation in the ICP27, ICP47, and/or UL55 genes.

In some embodiments, the recombinant herpes simplex virus genome comprises an inactivating mutation in the ICP4 gene (one or both copies). In some embodiments, the recombinant herpes simplex virus genome comprises an inactivating mutation in the ICP4 gene (one or both copies), and further comprises an inactivating mutation in the ICP0 (one or both copies), ICP22, ICP27, ICP47, UL41, and/or UL55 genes. In some embodiments, the recombinant herpes simplex virus genome comprises an inactivating mutation in the ICP4 gene (one or both copies), and an inactivating mutation in the ICP22 gene. In some embodiments, the recombinant herpes simplex virus genome comprises an inactivating mutation in the ICP4 gene (one or both copies), and an inactivating mutation in the UL41 gene. In some embodiments, the recombinant herpes simplex virus genome comprises an inactivating mutation in the ICP4 gene (one or both copies), an inactivating mutation in the ICP22 gene, and an inactivating mutation in the UL41 gene. In some embodiments, the inactivating mutation is a deletion of the coding sequence of the ICP4 (one or both copies), ICP22, and/or UL41 genes. In some embodiments, the recombinant herpes simplex virus genome further comprises an inactivating mutation in the ICP0 (one or both copies), ICP27, ICP47, and/or UL55 genes.

In some embodiments, the recombinant herpes simplex virus genome comprises an inactivating mutation in the ICP22 gene. In some embodiments, the recombinant herpes simplex virus genome comprises an inactivating mutation in the ICP22 gene, and further comprises an inactivating mutation in the ICP0 (one or both copies), ICP4 (one or both copies), ICP27, ICP47, UL41, and/or UL55 genes. In some embodiments, the recombinant herpes simplex virus genome comprises an inactivating mutation in the ICP22 gene, and an inactivating mutation UL41 gene. In some embodiments, the inactivating mutation is a deletion of the coding sequence of the ICP22 and/or UL41 genes. In some embodiments, the recombinant herpes simplex virus genome further comprises an inactivating mutation in the ICP0 (one or both copies), ICP4 (one or both copies), ICP27, ICP47, and/or UL55 genes.

In some embodiments, the recombinant herpes simplex virus genome comprises an inactivating mutation in the ICP27 gene. In some embodiments, the recombinant herpes simplex virus genome comprises an inactivating mutation in the ICP27 gene, and further comprises an inactivating mutation in the ICP0 (one or both copies), ICP4 (one or both copies), ICP22, ICP47, UL41, and/or UL55 genes. In some embodiments, the inactivating mutation is a deletion of the coding sequence of the ICP27 gene.

In some embodiments, the recombinant herpes simplex virus genome comprises an inactivating mutation in the ICP47 gene. In some embodiments, the recombinant herpes simplex virus genome comprises an inactivating mutation in the ICP47 gene, and further comprises an inactivating mutation in the ICP0 (one or both copies), ICP4 (one or both copies), ICP22, ICP27, UL41, and/or UL55 genes. In some embodiments, the inactivating mutation is a deletion of the coding sequence of the ICP47 gene.

In some embodiments, the recombinant herpes simplex virus genome comprises an inactivating mutation in the UL41 gene. In some embodiments, the recombinant herpes simplex virus genome comprises an inactivating mutation in the UL41 gene, and further comprises an inactivating mutation in the ICP0 (one or both copies), ICP4 (one or both copies), ICP22, ICP27, ICP47, and/or UL55 genes. In some embodiments, the inactivating mutation is a deletion of the coding sequence of the UL41 gene.

In some embodiments, the recombinant herpes simplex virus genome comprises an inactivating mutation in the UL55 gene. In some embodiments, the recombinant herpes simplex virus genome comprises an inactivating mutation in the UL55 gene, and further comprises an inactivating mutation in the ICP0 (one or both copies), ICP4 (one or both copies), ICP22, ICP27, ICP47, and/or UL41 genes. In some embodiments, the inactivating mutation is a deletion of the coding sequence of the UL55 gene.

In some embodiments, the recombinant herpes simplex virus genome comprises an inactivating mutation in (e.g., a deletion of) the internal repeat (Joint) region comprising the internal repeat long (IRL) and internal repeat short (IRs) regions. In some embodiments, inactivation (e.g., deletion) of the Joint region eliminates one copy each of the ICP4 and ICP0 genes. In some embodiments, inactivation (e.g., deletion) of the Joint region further inactivates (e.g., deletes) the promoter for the ICP22 and ICP47 genes. If desired, expression of one or both of these genes can be restored by insertion of an immediate early promoter into the recombinant herpes simplex virus genome (see e.g., Hill et al. (1995). Nature 375 (6530): 411-415; Goldsmith et al. (1998). J Exp Med 187 (3): 341-348). Without wishing to be bound by theory, it is believed that inactivating (e.g., deleting) the Joint region may contribute to the stability of the recombinant herpes simplex virus genome and/or allow for the recombinant herpes simplex virus genome to accommodate more and/or larger transgenes.

In some embodiments, the recombinant herpes simplex virus genome comprises an inactivating mutation in the ICP4 (one or both copies), ICP22, and ICP27 genes. In some embodiments, the recombinant herpes simplex virus genome comprises an inactivating mutation in the ICP4 (one or both copies), ICP27, and UL55 genes. In some embodiments, the recombinant herpes simplex virus genome comprises an inactivating mutation in the ICP4 (one or both copies), ICP22, ICP27, ICP47, and UL55 genes. In some embodiments, the inactivating mutation in the ICP4 (one or both copies), ICP27, and/or UL55 genes is a deletion of the coding sequence of the ICP4 (one or both copies), ICP27, and/or UL55 genes. In some embodiments, the inactivating mutation in the ICP22 and ICP47 genes is a deletion in the promoter region of the ICP22 and ICP47 genes (e.g., the ICP22 and ICP47 coding sequences are intact but are not transcriptionally active). In some embodiments, the recombinant herpes simplex virus genome comprises a deletion in the coding sequence of the ICP4 (one or both copies), ICP27, and UL55 genes, and a deletion in the promoter region of the ICP22 and ICP47 genes. In some embodiments, the recombinant herpes simplex virus genome further comprises an inactivating mutation in the ICP0 (one or both copies) and/or UL41 genes.

In some embodiments, the recombinant herpes simplex virus genome comprises an inactivating mutation in the ICP0 (one or both copies) gene. In some embodiments, the recombinant herpes simplex virus genome comprises an inactivating mutation in the ICP0 (one or both copies) and ICP4 (one or both copies) genes. In some embodiments, the recombinant herpes simplex virus genome comprises an inactivating mutation in the ICP0 (one or both copies), ICP4 (one or both copies), and ICP22 genes. In some embodiments, the recombinant herpes simplex virus genome comprises an inactivating mutation in the ICP0 (one or both copies), ICP4 (one or both copies), ICP22, and ICP27 genes. In some embodiments, the recombinant herpes simplex virus genome comprises an inactivating mutation in the ICP0 (one or both copies), ICP4 (one or both copies), ICP22, ICP27 and UL55 genes. In some embodiments, the inactivating mutation in the ICP0 (one or both copies), ICP4 (one or both copies), ICP22, ICP27 and/or UL55 genes comprises a deletion of the coding sequence of the ICP0 (one or both copies), ICP4 (one or both copies), ICP22, ICP27 and/or UL55 genes. In some embodiments, the recombinant herpes simplex virus genome further comprises an inactivating mutation in the ICP47 and/or the UL41 genes.

In some embodiments, a recombinant herpes simplex virus genome comprises one or more polynucleotides of the present disclosure within one, two, three, four, five, six, seven or more viral gene loci. Examples of suitable viral loci may include, without limitation, the ICP0 (one or both copies), ICP4 (one or both copies), ICP22, ICP27, ICP47, tk, UL41 and UL55 herpes simplex viral gene loci. In some embodiments, a recombinant herpes simplex virus genome comprises one or more polynucleotides of the present disclosure within one or both of the viral ICP4 gene loci (e.g., a recombinant virus comprising a polynucleotide encoding a polypeptide in one or both of the ICP4 loci). In some embodiments, a recombinant herpes simplex virus genome comprises one or more polynucleotides of the present disclosure within the viral ICP22 gene locus (e.g., a recombinant virus comprising a polynucleotide encoding a polypeptide in the ICP22 locus). In some embodiments, a recombinant herpes simplex virus genome comprises one or more polynucleotides of the present disclosure within the viral UL41 gene locus (e.g., a recombinant virus comprising a polynucleotide encoding a polypeptide in the UL41 locus). In some embodiments, a recombinant herpes simplex virus genome comprises one or more polynucleotides of the present disclosure within the viral ICP27 gene locus (e.g., a recombinant virus comprising a polynucleotide encoding a polypeptide in the ICP27 locus). In some embodiments, a recombinant herpes simplex virus genome comprises one or more polynucleotides of the present disclosure within the viral ICP47 gene locus (e.g., a recombinant virus comprising a polynucleotide encoding a polypeptide in the ICP47 locus). In some embodiments, a recombinant herpes simplex virus genome comprises one or more polynucleotides of the present disclosure within the viral UL55 gene locus (e.g., a recombinant virus comprising a polynucleotide encoding a polypeptide in the UL55 locus). In some embodiments, a recombinant herpes simplex virus genome comprises one or more polynucleotides of the present disclosure within the viral tk gene locus (e.g., a recombinant virus comprising a polynucleotide encoding a polypeptide in the tk locus).

In some embodiments, a recombinant herpes simplex virus genome comprises one or more polynucleotides of the present disclosure within one or both of the viral ICP4 gene loci, and one or more polynucleotides of the present disclosure within the viral ICP22 gene locus (e.g., a recombinant virus comprising a polynucleotide encoding a polypeptide in one or both of the ICP4 loci, and a polynucleotide encoding a polypeptide in the ICP22 locus). In some embodiments, a recombinant herpes simplex virus genome comprises one or more polynucleotides of the present disclosure within one or both of the viral ICP4 gene loci, and one or more polynucleotides of the present disclosure within the viral UL41 gene locus (e.g., a recombinant virus comprising a polynucleotide encoding a polypeptide in one or both of the ICP4 loci, and a polynucleotide encoding a polypeptide in the UL41 locus). In some embodiments, a recombinant herpes simplex virus genome comprises one or more polynucleotides of the present disclosure within the viral ICP22 gene locus, and one or more polynucleotides of the present disclosure within the viral UL41 gene locus (e.g., a recombinant virus comprising a polynucleotide encoding a polypeptide in the ICP22 locus, and a polynucleotide encoding a polypeptide in the UL41 locus). In some embodiments, a recombinant herpes simplex virus genome comprises one or more polynucleotides of the present disclosure within one or both of the viral ICP4 gene loci, one or more polynucleotides of the present disclosure within the viral ICP22 gene locus, and one or more polynucleotides of the present disclosure within the viral UL41 gene locus (e.g., a recombinant virus comprising a polynucleotide encoding a polypeptide in one or both of the ICP4 loci, a polynucleotide encoding a polypeptide in the ICP22 locus, and a polynucleotide encoding a polypeptide in the UL41 locus). In some embodiments, a recombinant herpes simplex virus genome comprises one or more polynucleotides of the present disclosure within one or both of the viral ICP4 gene loci, one or more polynucleotides of the present disclosure within the viral ICP22 gene locus, one or more polynucleotides of the present disclosure within the viral UL41 gene locus, one or more polynucleotides of the present disclosure within the viral ICP27 gene locus, one or more polynucleotides of the present disclosure within the viral ICP47 gene locus, one or more polynucleotides of the present disclosure within the viral tk gene locus, and/or one or more polynucleotides of the present disclosure within the viral UL55 gene locus.

In some embodiments, the recombinant herpes virus genome (e.g., a recombinant herpes simplex virus genome) has been engineered to decrease or eliminate expression of one or more herpes virus genes (e.g., one or more toxic herpes virus genes), such as one or both copies of the HSV ICP0 gene, one or both copies of the HSV ICP4 gene, the HSV ICP22 gene, the HSV UL41 gene, the HSV ICP27 gene, the HSV ICP47 gene, the HSV tk gene, the HSV UL55 gene, etc. In some embodiments, the recombinant herpes virus genome (e.g., a recombinant herpes simplex virus genome) has been engineered to reduce cytotoxicity of the recombinant genome (e.g., when introduced into a target cell), as compared to a corresponding wild-type herpes virus genome (e.g., a wild-type herpes simplex virus genome). In some embodiments, the target cell is a human cell (primary cells or a cell line derived therefrom). In some embodiments, the target cell is a cell of the eye (primary cells or a cell line derived therefrom). In some embodiments, cytotoxicity (e.g., in a target cell) of the recombinant genome (e.g., a recombinant herpes simplex virus genome) is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% as compared to a corresponding wild-type herpes virus genome (e.g., measuring the relative cytotoxicity of a recombinant ΔICP4 (one or both copies) herpes simplex virus genome vs. a wild-type herpes simplex virus genome in a target cell; measuring the relative cytotoxicity of a recombinant ΔICP4 (one or both copies)/ΔICP22 herpes simplex virus genome vs. a wild-type herpes simplex virus genome in a target cell, etc.). In some embodiments, cytotoxicity (e.g., in a target cell) of the recombinant herpes genome (e.g., a recombinant herpes simplex virus genome) is reduced by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more as compared to a corresponding wild-type herpes virus genome (e.g., measuring the relative cytotoxicity of a recombinant ΔICP4 (one or both copies) herpes simplex virus genome vs. a wild-type herpes simplex virus genome in a target cell; measuring the relative cytotoxicity of a recombinant ΔICP4 (one or both copies)/ΔICP22 herpes simplex virus genome vs. a wild-type herpes simplex virus genome in a target cell, etc.). Methods of measuring cytotoxicity are known to one of ordinary skill in the art, including, for example, through the use of vital dyes (formazan dyes), protease biomarkers, an MTT assay (or an assay using related tetrazolium salts such as XTT, MTS, water-soluble tetrazolium salts, etc.), measuring ATP content, etc.

In some embodiments, the recombinant genome (e.g., a recombinant herpes simplex virus genome) has been engineered to reduce its impact on target cell proliferation after exposure of a target cell to the recombinant genome, as compared to a corresponding wild-type genome (e.g., a wild-type herpes simplex virus genome). In some embodiments, the target cell is a human cell (primary cells or a cell line derived therefrom). In some embodiments, the target cell is a cell of the eye (primary cells or a cell line derived therefrom). In some embodiments, target cell proliferation after exposure to the recombinant genome is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% faster as compared to target cell proliferation after exposure to a corresponding wild-type genome (e.g., measuring the relative cellular proliferation after exposure to a recombinant ΔICP4 (one or both copies) herpes simplex virus genome vs. cellular proliferation after exposure to a wild-type herpes simplex virus genome in target cells; measuring the relative cellular proliferation after exposure to a recombinant ΔICP4 (one or both copies)/ΔICP22 herpes simplex virus genome vs. cellular proliferation after exposure to a wild-type herpes simplex virus genome in target cells, etc.). In some embodiments, target cell proliferation after exposure to the recombinant genome is at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, or at least about 1000-fold faster as compared to target cell proliferation after exposure to a corresponding wild-type genome (e.g., measuring the relative cellular proliferation after exposure to a recombinant ΔICP4 (one or both copies) herpes simplex virus genome vs. cellular proliferation after exposure to a wild-type herpes simplex virus genome in target cells; measuring the relative cellular proliferation after exposure to a recombinant ΔICP4 (one or both copies)/ΔICP22 herpes simplex virus genome vs. cellular proliferation after exposure to a wild-type herpes simplex virus genome in target cells, etc.). Methods of measuring cellular proliferation are known to one of ordinary skill in the art, including, for example, through the use of a Ki67 cell proliferation assay, a BrdU cell proliferation assay, etc.

A vector (e.g., herpes viral vector) may include one or more polynucleotides of the present disclosure in a form suitable for expression of the polynucleotide in a host cell. Vectors may include one or more regulatory sequences operatively linked to the polynucleotide to be expressed (e.g., as described above).

In some embodiments, the present disclosure relates to one or more heterologous polynucleotides (e.g., a bacterial artificial chromosome (BAC)) comprising any of the recombinant nucleic acids described herein.

In some embodiments, a recombinant nucleic acid (e.g., a recombinant herpes simplex virus genome) of the present disclosure comprises one or more of the polynucleotides described herein inserted in any orientation in the recombinant nucleic acid. If the recombinant nucleic acid comprises two or more polynucleotides described herein (e.g., two or more, three or more, etc.), the polynucleotides may be inserted in the same orientation or opposite orientations to one another. Without wishing to be bound be theory, incorporating two polynucleotides (e.g., two transgenes) into a recombinant nucleic acid (e.g., a vector) in an antisense orientation may help to avoid read-through and ensure proper expression of each polynucleotide.

In some embodiments, a recombinant nucleic of the present disclosure does not comprise a polynucleotide encoding a Collagen alpha-1 (VII) chain polypeptide (COL7). In some embodiments, a recombinant nucleic of the present disclosure does not comprise a polynucleotide encoding a Lysyl hydroxylase 3 polypeptide (LH3). In some embodiments, a recombinant nucleic of the present disclosure does not comprise a polynucleotide encoding a Keratin type I cytoskeletal 17 polypeptide (KRT17). In some embodiments, a recombinant nucleic of the present disclosure does not comprise a polynucleotide encoding a transglutaminase (TGM) polypeptide (e.g., a human transglutaminase polypeptide such as a human TGM1 polypeptide and/or a human TGM5 polypeptide). In some embodiments, a recombinant nucleic of the present disclosure does not comprise a polynucleotide encoding a cosmetic protein (e.g., collagen proteins, fibronectins, elastins, lumicans, vitronectins/vitronectin receptors, laminins, neuromodulators, fibrillins, additional dermal extracellular matrix proteins, etc.). In some embodiments, a recombinant nucleic of the present disclosure does not comprise a polynucleotide encoding an antibody (e.g., a full-length antibody, an antibody fragment, etc.). In some embodiments, a recombinant nucleic of the present disclosure does not comprise a polynucleotide encoding a Serine Protease Inhibitor Kazal-type (SPINK) polypeptide (e.g., a human SPINK polypeptide, such as a SPINK5 polypeptide). In some embodiments, a recombinant nucleic of the present disclosure does not comprise a polynucleotide encoding a filaggrin or filaggrin 2 polypeptide (e.g., a human filaggrin or filaggrin 2 polypeptide). In some embodiments, a recombinant nucleic of the present disclosure does not comprise a polynucleotide encoding a Cystic Fibrosis Transmembrane Conductance Regulator (CFTR) polypeptide (e.g., a human CFTR polypeptide). In some embodiments, a recombinant nucleic of the present disclosure does not comprise a polynucleotide encoding an ichthyosis-associated polypeptide (e.g., an ATP-binding cassette sub-family A member 12 polypeptide, a 1-acylglycerol-3-phosphate O-acyltransferase ABHD5 polypeptide, an Aldehyde dehydrogenase family 3 member A2 polypeptide, an Arachidonate 12-lipoxygenase 12R-type polypeptide, a Hydroperoxide isomerase ALOXE3 polypeptide, an AP-1 complex subunit sigma-1A polypeptide, an Arylsulfatase E polypeptide, a Caspase-14 polypeptide, a Corneodesmosin polypeptide, a Ceramide synthase 3 polypeptide, a Carbohydrate sulfotransferase 8 polypeptide, a Claudin-1 polypeptide, a Cystatin-A polypeptide, a Cytochrome P450 4F22 polypeptide, a 3-beta-hydroxysteroid-Delta (8), Delta (7)-isomerase polypeptide, an Elongation of very long chain fatty acids protein 4 polypeptide, a Filaggrin polypeptide, a Filaggrin 2 polypeptide, a Gap junction beta-2 polypeptide, a Gap junction beta-3 polypeptide, a Gap junction beta-4 polypeptide, a Gap junction beta-6 polypeptide, a 3-ketodihydrosphingosine reductase polypeptide, a Keratin, type II cytoskeletal 1 polypeptide, a Keratin, type II cytoskeletal 2 epidermal polypeptide, a Keratin, type I cytoskeletal 9 polypeptide, a Keratin, type I cytoskeletal 10 polypeptide, a Lipase member N polypeptide, a Loricrin polypeptide, a Membrane-bound transcription factor site-2 protease polypeptide, a Magnesium transporter NIPA4 polypeptide, a Sterol-4-alpha-carboxylate 3-dehydrogenase, decarboxylating polypeptide, a Peroxisomal targeting signal 2 receptor polypeptide, a D-3-phosphoglycerate dehydrogenase polypeptide, a Phytanoyl-CoA dioxygenase, peroxisomal polypeptide, Patatin-like phospholipase domain-containing protein 1 polypeptide, a Proteasome maturation protein polypeptide, a Phosphoserine aminotransferase polypeptide, a Short-chain dehydrogenase/reductase family 9C member 7 polypeptide, a Serpin B8 polypeptide, a Long-chain fatty acid transport protein 4 polypeptide, a Synaptosomal-associated protein 29 polypeptide, a Suppressor of tumorigenicity 14 protein polypeptide, a Steryl-sulfatase polypeptide, a Vacuolar protein sorting-associated protein 33B polypeptide, and a CAAX prenyl protease 1 homolog polypeptide). In some embodiments, a recombinant nucleic of the present disclosure does not comprise a polynucleotide encoding a Collagen alpha-1 (VII) chain polypeptide, a Lysyl hydroxylase 3 polypeptide, a Keratin type I cytoskeletal 17 polypeptide, and/or any chimeric polypeptides thereof. In some embodiments, a recombinant nucleic of the present disclosure does not comprise a polynucleotide encoding a Collagen alpha-1 (VII) chain polypeptide, a Lysyl hydroxylase 3 polypeptide, a Keratin type I cytoskeletal 17 polypeptide, a transglutaminase (TGM) polypeptide, a filaggrin polypeptide, a cosmetic protein, an antibody, a SPINK polypeptide, a CFTR polypeptide, an ichthyosis-associated polypeptide, an Alpha-1-antitrypsin polypeptide, a Sodium-dependent phosphate transport protein 2B polypeptide, a Dynein heavy chain 5 axonemal polypeptide, a Dynein heavy chain 11 axonemal polypeptide, a Coiled-coil domain-containing protein 39 polypeptide, a Dynein intermediate chain 1 axonemal polypeptide, a Coiled-coil domain-containing protein 40 polypeptide, a Coiled-coil domain containing protein 103 polypeptide, a Sperm-associated antigen 1 polypeptide, a Zinc finger MYND domain-containing protein 10 polypeptide, an Armadillo repeat containing protein 4 polypeptide, a Coiled-coil domain-containing protein 151 polypeptide, a Dynein intermediate chain 2 axonemal polypeptide, a Radial spoke head 1 homolog polypeptide, a Coiled-coil domain-containing protein 114 polypeptide, a Radial spoke head protein 4 homolog A polypeptide, a Dynein assembly factor 1 axonemal polypeptide, a Dynein assembly factor 2 axonemal polypeptide, a Leucine-rich repeat-containing protein 6 polypeptide, a Pulmonary surfactant-associated protein B polypeptide, a Pulmonary surfactant-associated protein C polypeptide, a Homeobox protein Nkx-2.1 polypeptide, an ATP-binding cassette sub-family A member 3 polypeptide, a Cytokine receptor common subunit beta polypeptide, a Granulocyte-macrophage colony-stimulating factor receptor subunit alpha polypeptide, a Bone morphogenetic protein receptor type-2 polypeptide, a Sarcoplasmic/endoplasmic reticulum calcium ATPase 2 polypeptide, a serine/threonine-protein kinase receptor R3 polypeptide, an Endoglin polypeptide, a Mothers against decapentaplegic homolog 9 polypeptide, a Caveolin-1 polypeptide, a Potassium channel subfamily K member 3 polypeptide, an elF-2-alpha kinase GCN2 polypeptide, a Pulmonary surfactant-associated protein A2 polypeptide, a Telomerase reverse transcriptase polypeptide, a Dyskerin polypeptide, a Regulator of telomere elongation helicase 1 polypeptide, a Poly(A)-specific ribonuclease PARN polypeptide, a TERF1-interacting nuclear factor 2 polypeptide, an H/ACA ribonucleoprotein complex non-core subunit NAF1 polypeptide, a Mucin-5B polypeptide, a Desmoplakin polypeptide, a CST complex subunit STN1 polypeptide, a Dipeptidyl peptidase 9 polypeptide, and/or any chimeric polypeptides thereof.

In some embodiments, a recombinant nucleic acid of the present disclosure does not comprise a polynucleotide encoding an immunomodulatory polypeptide. In some embodiments, a recombinant nucleic acid of the present disclosure does not comprise a polynucleotide encoding a cytokine polypeptide and/or a chemokine polypeptide. In some embodiments, a recombinant nucleic acid of the present disclosure does not comprise a polynucleotide encoding a cytokine polypeptide. In some embodiments, a recombinant nucleic acid of the present disclosure does not comprise a polynucleotide encoding an IL-1 polypeptide, an IL-2 polypeptide, an IL-7 polypeptide, an IL-12 polypeptide, an IL-13 polypeptide, an IL-15 polypeptide, an IL-17 polypeptide, an IL-18 polypeptide, an IL-28 polypeptide, an IL-32 polypeptide, an IL-33 polypeptide, an IL-34 polypeptide, a TNFα polypeptide, an IFNγ polypeptide, a G-CSF polypeptide, a GM-CSF polypeptide, and/or any chimeric polypeptides thereof.

In some embodiments, a recombinant nucleic acid of the present disclosure does not comprise a polynucleotide encoding a chemokine polypeptide. In some embodiments, a recombinant nucleic acid of the present disclosure does not comprise a polynucleotide encoding a CXCL1 polypeptide, a CXCL2 polypeptide, a CXCL8 polypeptide, a CXCL9 polypeptide, a CXCL11 polypeptide, a CXCL16 polypeptide, a CCL2 polypeptide, a CCL3 polypeptide, a CCL4 polypeptide, a CCL5 polypeptide, a CCL11 polypeptide, and/or any chimeric polypeptides thereof.

### IV. Viruses

Certain aspects of the present disclosure relate to viruses comprising any of the polynucleotides and/or recombinant nucleic acids described herein. In some embodiments, the virus is capable of infecting one or more target cells of a subject (e.g., a human). In some embodiments, the virus is suitable for delivering the polynucleotides and/or recombinant nucleic acids into one or more target cells of a subject (e.g., a human). In some embodiments, the one or more target cells are human cells. In some embodiments, the one or more target cells are one or more cells of the eye (e.g., anterior chamber; posterior chamber; ciliary body; corneal epithelial cell; rod cell; cone cell; photoreceptor; retinal pigment epithelial cell; retinal ganglion cell; bipolar cell; horizontal cell; muller cell; amacrine cell; etc.).

Any suitable virus known in the art may be used, including, for example, adenovirus, adeno-associated virus, retrovirus, lentivirus, sendai virus, papillomavirus, herpes virus (e.g., a herpes simplex virus), vaccinia virus, and/or any hybrid or derivative viruses thereof. In some embodiments, the virus is attenuated. In some embodiments, the virus is replication competent. In some embodiments, the virus is replication defective. In some embodiments, the virus is not oncolytic. In some embodiments, the virus has been modified to alter its tissue tropism relative to the tissue tropism of a corresponding unmodified, wild-type virus. In some embodiments, the virus has reduced cytotoxicity (e.g., in a target cell) as compared to a corresponding wild-type virus. Methods of producing a virus comprising recombinant nucleic acids are well known to one of ordinary skill in the art.

In some embodiments, the virus is a member of the Herpesviridae family of DNA viruses, including, for example, a herpes simplex virus, a varicella zoster virus, a human cytomegalovirus, a herpesvirus 6A, a herpesvirus 6B, a herpesvirus 7, an Epstein-Barr virus, and a Kaposi's sarcoma-associated herpesvirus, etc. In some embodiments, the herpes virus is attenuated. In some embodiments, the herpes virus is replication defective. In some embodiments, the herpes virus is replication competent. In some embodiments, the herpes virus has been engineered to reduce or eliminate expression of one or more herpes virus genes (e.g., one or more toxic herpes virus genes). In some embodiments, the herpes virus has reduced cytotoxicity as compared to a corresponding wild-type herpes virus. In some embodiments, the herpes virus is not oncolytic.

In some embodiments, the virus is a herpes simplex virus. Herpes simplex viruses comprising recombinant nucleic acids may be produced by a process disclosed, for example, in WO2015/009952, WO2017/176336, WO2019/200163, and/or WO2019/210219. In some embodiments, the herpes simplex virus is attenuated. In some embodiments, the herpes simplex virus is replication defective. In some embodiments, the herpes simplex virus is replication competent. In some embodiments, the herpes simplex virus has been engineered to reduce or eliminate expression of one or more herpes simplex virus genes (e.g., one or more toxic herpes simplex virus genes). In some embodiments, the herpes simplex virus has reduced cytotoxicity as compared to a corresponding wild-type herpes simplex virus. In some embodiments, the herpes simplex virus is not oncolytic. In some embodiments, the herpes simplex virus is an HSV-1, an HSV-2, or any derivatives thereof. In some embodiments, the herpes simplex virus is an HSV-1 virus. In some embodiments, the HSV-1 is attenuated. In some embodiments, the HSV-1 is replication defective. In some embodiments, the HSV-1 is replication competent. In some embodiments, the HSV-1 has been engineered to reduce or eliminate expression of one or more HSV-1 genes (e.g., one or more toxic HSV-1 genes). In some embodiments, the HSV-1 has reduced cytotoxicity as compared to a corresponding wild-type HSV-1. In some embodiments, the HSV-1 is not oncolytic.

In some embodiments, the herpes simplex virus has been modified to alter its tissue tropism relative to the tissue tropism of an unmodified, wild-type herpes simplex virus. In some embodiments, the herpes simplex virus comprises a modified envelope. In some embodiments, the modified envelope comprises one or more (e.g., one or more, two or more, three or more, four or more, etc.) mutant herpes simplex virus glycoproteins. Examples of herpes simplex virus glycoproteins may include, but are not limited to, the glycoproteins gB, gC, gD, gH, and gL. In some embodiments, the modified envelope alters the herpes simplex virus tissue tropism relative to a wild-type herpes simplex virus.

In some embodiments, the transduction efficiency (in vitro and/or in vivo) of a virus of the present disclosure (e.g., a herpes virus such as a herpes simplex virus) for one or more target cells (e.g., one or more cells of the eye) is at least about 25%. For example, the transduction efficiency of the virus for one or more target cells may be at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 99.5%, or more. In some embodiments, the virus is a herpes simplex virus and the transduction efficiency of the virus for one or more target cells (e.g., one or more cells of the eye) is about 85% to about 100%. In some embodiments, the virus is a herpes simplex virus and the transduction efficiency of the virus for one or more target cells (e.g., one or more cells of the eye) is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100%. Methods of measuring viral transduction efficiency in vitro or in vivo are well known to one of ordinary skill in the art, including, for example, qPCR analysis, deep sequencing, western blotting, fluorometric analysis (such as fluorescent in situ hybridization (FISH), fluorescent reporter gene expression, immunofluorescence, FACS), etc.

In some embodiments, provided herein are recombinant viruses, which may or may not be pseudotyped, that produce one or more therapeutic polypeptides for the treatment of an eye condition or disease. In some embodiments, the one or more therapeutic polypeptides produced by the recombinant viruses described herein mediate or enhance a treatment of an eye condition or disease. The present disclosure further provides therapeutic compositions comprising the recombinant viruses and methods of use in the treatment of an eye condition or disease.

### V. Pharmaceutical Compositions and Formulations

Certain aspects of the present disclosure relate to pharmaceutical compositions or formulations comprising any of the recombinant nucleic acids (e.g., a recombinant herpes virus genome) and/or viruses (e.g., a herpes virus comprising a recombinant genome) described herein (such as a herpes simplex virus comprising a recombinant herpes simplex virus genome), and a pharmaceutically acceptable excipient or carrier.

In some embodiments, the pharmaceutical composition or formulation comprises any one or more of the viruses (e.g., herpes viruses) described herein. In some embodiments, the pharmaceutical composition or formulation comprises from about 10⁴ to about 10¹² plaque forming units (PFU)/ml of the virus. For example, the pharmaceutical composition or formulation may comprise from about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10⁴ to about 10⁹, about 10⁵ to about 10⁹, about 10⁶ to about 10⁹, about 10⁷ to about 10⁹, about 10⁸ to about 10⁹, about 10⁴ to about 10⁸, about 10⁵ to about 10⁸, about 10⁶ to about 108, about 10⁷ to about 10⁸, about 10⁴ to about 10⁷, about 10⁵ to about 10⁷, about 10⁶ to about 10⁷, about 10⁴ to about 10⁶, about 10⁵ to about 10⁶, or about 10⁴ to about 10⁵ PFU/mL of the virus. In some embodiments, the pharmaceutical composition or formulation comprises about 10⁴, about 10⁵, about 10⁶, about 10⁷, about 10⁸, about 10⁹, about 10¹⁰, about 10¹¹, or about 10¹² PFU/mL of the virus.

Pharmaceutical compositions and formulations can be prepared by mixing the active ingredient(s) (such as a recombinant nucleic acid and/or a virus) having the desired degree of purity with one or more pharmaceutically acceptable carriers or excipients. Pharmaceutically acceptable carriers or excipients are generally nontoxic to recipients at the dosages and concentrations employed, and may include, but are not limited to: buffers (such as phosphate, citrate, acetate, and other organic acids); antioxidants (such as ascorbic acid and methionine); preservatives (such as octadecyldimethylbenzyl ammonium chloride, benzalkonium chloride, benzethonium chloride, phenol, butyl or benzyl alcohol, alkyl parabens, catechol, resorcinol, cyclohexanol, 3-pentanol, and m-cresol); amino acids (such as glycine, glutamine, asparagine, histidine, arginine, or lysine); low molecular weight (less than about 10 residues) polypeptides; proteins (such as serum albumin, gelatin, or immunoglobulins); polyols (such as glycerol, e.g., formulations including 1%, 2%, 3%, 4%, 5%, 6%, 7%, 8%, 9%, 10%, 11%, 12%, 13%, 14%, 15%, etc. glycerol); hydrophilic polymers (such as polyvinylpyrrolidone); monosaccharides, disaccharides, and other carbohydrates (including glucose, mannose, or dextrins); chelating agents (such as EDTA); sugars (such as sucrose, mannitol, trehalose, or sorbitol); salt-forming counter-ions (such as sodium); metal complexes (such as Zn-protein complexes); and/or non-ionic surfactants (such as polyethylene glycol (PEG)). A thorough discussion of pharmaceutically acceptable carriers is available in REMINGTON'S PHARMACEUTICAL SCIENCES (Mack Pub. Co., N.J. 1991).

In some embodiments, the pharmaceutical composition or formulation is suitable for an eye drop, an ointment, a paste, a cream, a suspension, an emulsion, a fatty ointment, a gel, a bioadhesive gel, a powder, a lotion, a solution, and/or a spray.

In some embodiments, the pharmaceutical composition or formulation comprises one or more lipid (e.g., cationic lipid) carriers. In some embodiments, the pharmaceutical composition or formulation comprises one or more nanoparticle carriers. Nanoparticles are submicron (less than about 1000 nm) sized drug delivery vehicles that can carry encapsulated drugs (such as synthetic small molecules, proteins, peptides, cells, viruses, and nucleic acid-based biotherapeutics) for rapid or controlled release. A variety of molecules (e.g., proteins, peptides, recombinant nucleic acids, etc.) can be efficiently encapsulated in nanoparticles using processes well known in the art. In some embodiments, a molecule “encapsulated” in a nanoparticle may refer to a molecule (such as a virus) that is contained within the nanoparticle or attached to and/or associated with the surface of the nanoparticle, or any combination thereof. Nanoparticles for use in the compositions or formulations described herein may be any type of biocompatible nanoparticle known in the art, including, for example, nanoparticles comprising poly(lactic acid), poly(glycolic acid), PLGA, PLA, PGA, and any combinations thereof (see e.g., Vauthier et al. Adv Drug Del Rev. (2003) 55:519-48; US2007/0148074; US2007/0092575; US2006/0246139; U.S. Pat. Nos. 5,753,234; 7,081,483; and WO2006/052285).

In some embodiments, the pharmaceutically acceptable carrier or excipient may be adapted for or suitable for any administration route known in the art, including, for example, ocular, subretinal, intraocular, intravitreal, topical, subcutaneous, subconjunctival, subtenon, subtenon capsule, intracameral, retrobulbar, systemic, parenteral, periocular, juxtascleral, anterior juxtascleral, posterior juxtascleral, oral, peribulbar, or suprachoroidal administration.

In some embodiments, the pharmaceutically acceptable carrier or excipient may be adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, suprachoroidal, transmucosal, intraarticular, by implantation, by inhalation, intrathecal, intraventricular, and/or intranasal administration.

In some embodiments, the pharmaceutical composition or formulation is adapted for or suitable for any administration route known in the art, including, for example, intravenous, intramuscular, subcutaneous, cutaneous, oral, intranasal, intratracheal, sublingual, buccal, topical, transdermal, intradermal, intraperitoneal, intraorbital, intravitreal, subretinal, suprachoroidal, transmucosal, intraarticular, by implantation, by inhalation, intrathecal, intraventricular, or intranasal administration.

In some embodiments, the pharmaceutical composition or formulation further comprises one or more additional components. Examples of additional components may include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; hydroxymethylcellulose; polyvinylpyrrolidone; sweetenings; flavorings; perfuming agents; colorants; moisturizers; sunscreens; antibacterial agents; agents able to stabilize polynucleotides or prevent their degradation, and the like. In some embodiments, the pharmaceutical composition or formulation comprises a methylcellulose gel (e.g., hydroxypropyl methylcellulose, carboxy methylcellulose, etc.). In some embodiments, the pharmaceutical composition or formulation comprises a phosphate buffer. In some embodiments, the pharmaceutical composition or formulation comprises glycerol (e.g., at about 1%, about 2%, about 3%, about 4%, about 5%, about 6%, about 7%, about 8%, about 9%, about 10%, about 11%, about 12%, about 13%, about 14%, about 15%, etc.). In some embodiments, the pharmaceutical composition or formulation comprises a phosphate buffer and glycerol.

Pharmaceutical compositions and formulations to be used for in vivo administration are generally sterile. Sterility may be readily accomplished, e.g., by filtration through sterile filtration membranes.

In some embodiments, any of the recombinant nucleic acids, viruses, and/or pharmaceutical compositions or formulations described herein may be used to deliver one or more polynucleotides encoding a polypeptide into one or more cells of a subject (e.g., one or more cells of the eye of the subject). In some embodiments, the subject suffers from an eye condition or disease. In some embodiments, any of the recombinant nucleic acids, viruses, and/or pharmaceutical compositions or formulations described herein may be used in the treatment of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and/or calcific band keratopathy.

In some embodiments, the one or more transgenes include a gene expressed in a photoreceptor in a subject.

In some embodiments, the disease or condition is Stargardt disease. In some embodiments, the one or more symptoms include central vision loss starting in childhood or early adolescence. In some embodiments, the disease or condition is autosomal recessive. In some embodiments, the disease or condition involves one or more mutations in ABCA4. In some embodiments, the disease or condition has a prevalence of about 1 in 10,000. Mutations in ABCA4 also cause cone-rod dystrophy (loss of visual sharpness, photophobia, impaired color vision) that worsens over time and is also autosomal recessive inheritance. In some embodiments, the disease or condition involves one or more mutations in ELOVL4. For instance, Stargardt disease can also less often be caused by mutations in the ELOVL4 gene.

In some embodiments, the disease or condition is retinitis pigmentosa. In some embodiments, the one or more symptoms include a broad spectrum of severity, some starting in childhood others later in adulthood depending on the mutation. In some embodiments, the one or more symptoms include night blindness, peripheral vision loss, and/or eventually symptoms affecting central vision. In some embodiments, the disease or condition is caused by one or more gene mutations. In some embodiments, the disease or condition involves ˜60 gene mutations. In some embodiments, the disease or condition involves autosomal dominant mutation(s) in RHO, which can account for about 20-30% of cases. In some embodiments, the disease or condition involves autosomal recessive mutation(s) in USH2A (Usher syndrome type II), which can account for about 10-15% of cases and can have a prevalence of about ˜4 in 100,000). In some embodiments, the one or more symptoms start in late adolescence/early adulthood. In some embodiments, the disease or condition has a prevalence of about 1 in 4,000.

In some embodiments, the one or more transgenes include a gene expressed in a retinal pigment epithelium (RPE) cell in a subject.

In some embodiments, the disease or condition is Leber congenital amaurosis (LCA). In some embodiments, the one or more symptoms include severe visual impairment starting in infancy. In some embodiments, the disease or condition is autosomal recessive. In some embodiments, the disease or condition has a prevalence of about 2 in 100,000. In some embodiments, the disease or condition involves one or more mutations in RPE65 and/or CEP290. Other major gene mutation populations are genes expressed primarily in photoreceptors and not in RPE cells.

In some embodiments, the disease or condition is Fundus albipunctatus. In some embodiments, the one or more symptoms include night blindness and delayed dark adaptation. In some embodiments, the disease or condition is autosomal recessive. In some embodiments, the disease or condition is a type of Congenital stationary night blindness. In some embodiments, the disease or condition involves one or more mutations in RDH5 gene in RPE cells.

In some embodiments, the disease or condition is Retinitis punctata albescense. In some embodiments, the one or more symptoms can start in childhood and can include night blindness. In some embodiments, the disease or condition is autosomal recessive. In some embodiments, the disease or condition has a prevalence of about 1% of patients with autosomal recessive RCD (1 in 800,000 individuals worldwide). In some embodiments, the disease or condition involves one or more mutations in RLBP1.

In some embodiments, the disease or condition is inherited retinal dystrophies (IRD). In some embodiments, the disease or condition is IRD caused by one or more MERTK receptor mutations. In some embodiments, the disease or condition is autosomal recessive. In some embodiments, the disease or condition has a prevalence of about 2% of inherited retinal dystrophies (IRD) patients, where IRD has a prevalence of about 1 in 2000).

In some embodiments, the disease or condition or a symptom thereof can include any one or more of: Stargardt disease; Leber Congenital Amaurosis; Early-Onset Severe Retinal Dystrophy; Retinitis Pigmentosa; Nonsyndromic Retinitis Pigmentosa; Usher Syndrome Types 1-3; Bietti Crystalline Corneoretinal Dystrophy; Congenital Stationary Night Blindness; Fundus Albipunctatus; Retinitis Punctata Albescense; Choroideremia; Achromatopsia; Retinal manifestations of Alstrom Syndrome; Bardet-Biedl Syndrome; Joubert Syndrome; Gyrate Atrophy Of The Choroid and Retina; X-Linked Congenital Retinoschisis; Progressive Bifocal Chorioretinal Atrophy; Retinoblastoma; Uveitis; Diabetic Retinopathy; Diabetic Macular Edema; Persistent Corneal Epithelial Defect; Neurotrophic Keratitis; Chronic Dry Eye; Wet AMD; Dry AMD; Geographic Atopy; and Glaucoma.

In some embodiments, provided herein are compositions, methods, and uses for gene therapy for ocular disorders. In some embodiments, the recombinant HSV genome or the HSV can be used for topical administration.

### VI. Methods

Certain aspects of the present disclosure relate to a method of delivering an polypeptide to one or more cells of a subject (e.g., one or more cells of the eye, such as anterior chamber; posterior chamber; ciliary body; corneal epithelial cell; rod cell; cone cell; photoreceptor; retinal pigment epithelial cell; retinal ganglion cell; bipolar cell; horizontal cell; muller cell; amacrine cell; etc.) comprising administering to the subject a pharmaceutical composition comprising (a) any of the viruses described herein (e.g., a herpes simplex virus, such as an HSV-1) comprising any of the recombinant nucleic acids described herein (e.g., a recombinant herpes simplex virus genome, such as a recombinant HSV-1 genome) comprising one or more polynucleotides encoding the polypeptide, and a pharmaceutically acceptable carrier. In some embodiments, the pharmaceutical composition is administered via injection, via injection to the eye, via subretinal injection, via intraocular injection, via intravitreal injection, via suprachoroidal injection, via intracameral injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally to the subject. In some embodiments, the herpes virus or pharmaceutical composition is administered using an eye drop, an ointment, a paste, a cream, a suspension, an emulsion, a fatty ointment, a gel, a bioadhesive gel, a powder, a lotion, a solution, or a spray. In some embodiments, the herpes virus (e.g., the herpes simplex virus) is replication competent. In some embodiments, the herpes virus (e.g., the herpes simplex virus) is replication defective. In some embodiments, the herpes virus (e.g., the herpes simplex virus) is not oncolytic.

In some embodiments, the subject is a human. In some embodiments, the subject suffers from an eye condition or disease. In some embodiments, the eye condition or disease is selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital night blindness, congenital stationary night blindness, severe congenital stationary night blindness, recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and calcific band keratopathy. In some embodiments, the subject suffers from Stargardt disease. In some embodiments, the subject suffers from neurotrophic keratitis. In some embodiments, the subject suffers from Leber congenital amaurosis.

In some embodiments, administration of the recombinant nucleic acid, virus, medicament, and/or pharmaceutical composition or formulation to the subject increases the polypeptide levels (transcript or protein levels) by at least about 2-fold in one or more contacted or treated cells of the subject, as compared to the endogenous levels of the polypeptide in one or more corresponding untreated cells in the subject. For example, administration of the recombinant nucleic acid, virus, medicament, and/or pharmaceutical composition or formulation may increase the polypeptide levels (transcript or protein levels) by at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, at least about 100-fold, at least about 250-fold, at least about 500-fold, at least about 750-fold, at least about 1000-fold, or more in one or more contacted or treated cells of the subject, as compared to the endogenous levels of the polypeptide in one or more corresponding untreated cells in the subject. In some embodiments, the one or more contacted or treated cells are one or more cells of the eye. Methods of measuring transcript or protein levels from a sample are well known to one of ordinary skill in the art, including, for example, qPCR, western blot, mass spectrometry, etc.

Other aspects of the present disclosure relate to a method of providing prophylactic, palliative, or therapeutic relief to one or more signs or symptoms of an eye condition or disease in a subject in need thereof comprising administering to the subject an effective amount of any of the recombinant nucleic acids, viruses, medicaments, and/or pharmaceutical compositions or formulations described herein. In some embodiments, the subject is a human. In some embodiments, the subject suffers from an eye condition or disease. In some embodiments, the eye condition or disease is selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital night blindness, congenital stationary night blindness, severe congenital stationary night blindness, recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline I dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and calcific band keratopathy. In some embodiments, the subject suffers from Stargardt disease. In some embodiments, the subject suffers from neurotrophic keratitis. In some embodiments, the subject suffers from Leber congenital amaurosis.

The recombinant nucleic acids, viruses, medicaments, and/or pharmaceutical compositions or formulations described herein may be administered by any suitable method or route known in the art, including, without limitation, via injection, via injection to the eye, via subretinal injection, via intraocular injection, via intravitreal injection, via suprachoroidal injection, via intracameral injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, suprachoroidally, intranasally, intratracheally, sublingually, buccally, rectally, via inhalation, transdermally, subcutaneously, intradermally, intravenously, intraarterially, intramuscularly, intracardially, intraosseously, intraperitoneally, transmucosally, vaginally, intravitreally, intraorbitally, subretinally, intraarticularly, peri-articularly, locally, epicutaneously, or any combinations thereof. The present disclosure thus encompasses methods of delivering any of the recombinant nucleic acids, viruses, medicaments, or pharmaceutical compositions or formulations described herein to an individual (e.g., an individual having an eye condition or disease). In some embodiments, the recombinant nucleic acids, viruses, medicaments, and/or pharmaceutical compositions or formulations described herein are administered via injection, via injection to the eye, via subretinal injection, via intraocular injection, via intravitreal injection, via suprachoroidal injection, via intracameral injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally.

Methods of delivering drugs to the eye are generally known to one of ordinary skill in the art (see e.g., Gaudana et al. Ocular Drug Delivery, The AAPS Journal, Vol. 12, No. 3, September 2010).

In some embodiments, the recombinant nucleic acids, viruses, medicaments, and/or compositions or formulations are delivered to the eye by an eye drop, an ointment, a paste, a cream, a suspension, an emulsion, a fatty ointment, a gel, a bioadhesive gel, a powder, a lotion, a solution, or a spray.

In some embodiments, the recombinant nucleic acids, viruses, medicaments, and/or pharmaceutical compositions or formulations are administered once to the subject. In some embodiments, the recombinant nucleic acids, viruses, medicaments, and/or pharmaceutical compositions are administered at least twice (e.g., at least 2 times, at least 3 times, at least 4 times, at least 5 times, at least 10 times, etc.) to the subject. In some embodiments, at least about 1 hour (e.g., at least about 1 hour, at least about 6 hours, at least about 12 hours, at least about 18 hours, at least about 1 day, at least about 2 days, at least about 3 days, at least about 4 days, at least about 5 days, at least about 6 days, at least about 7 days, at least about 15 days, at least about 20 days, at least about 30 days, at least about 40 days, at least about 50 days, at least about 60 days, at least about 70 days, at least about 80 days, at least about 90 days, at least about 100 days, at least about 120 days, etc.) pass between administrations (e.g., between the first and second administrations, between the second and third administrations, etc.). In some embodiments, the recombinant nucleic acids, viruses, medicaments, and/or pharmaceutical compositions or formulations are administered one, two, three, four, five or more times per day to the subject. In some embodiments, the recombinant nucleic acids, viruses, medicaments, and/or pharmaceutical compositions or formulations are administered one, two, three, four, five or more times per month to the subject.

### VII. Host Cells

Certain aspects of the present disclosure relate to one or more host cells comprising any of the recombinant nucleic acids described herein. Any suitable host cell (prokaryotic or eukaryotic) known in the art may be used, including, for example: prokaryotic cells including eubacteria, such as Gram-negative or Gram-positive organisms, for example Enterobacteriaceae such as Escherichia (e.g., E. coli), Enterobacter, Erminia, Klebsiella, Proteus, Salmonella (e.g., S. typhimurium), Serratia (e.g., S. marcescans), and Shigella, as well as Bacilli such as B. subtilis and B. licheniformis; fungal cells (e.g., S. cerevisiae); insect cells (e.g., S2 cells, etc.); and mammalian cells, including monkey kidney CV1 line transformed by SV40 (COS-7, ATCC CRL 1651), human embryonic kidney line (293 or 293 cells subcloned for growth in suspension culture), baby hamster kidney cells (BHK, ATCC CCL 10), mouse Sertoli cells (TM4), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TRI cells, MRC 5 cells, FS4 cells, human hepatoma line (Hep G2), Chinese hamster ovary (CHO) cells, including DHFR″ CHO cells, and myeloma cell lines such as NSO and Sp2/0. In some embodiments, the host cell is a human or non-human primate cell. In some embodiments, the host cells are cells from a cell line. Examples of suitable host cells or cell lines may include, but are not limited to, 293, HeLa, SH-Sy5y, Hep G2, CACO-2, A549, L929, 3T3, K562, CHO-K1, MDCK, HUVEC, Vero, N20, COS-7, PSN1, VCaP, CHO cells, and the like.

In some embodiments, the recombinant nucleic acid is a herpes simplex viral vector. In some embodiments, the recombinant nucleic acid is a herpes simplex virus amplicon. In some embodiments, the recombinant nucleic acid is an HSV-1 amplicon or HSV-1 hybrid amplicon. In some embodiments, a host cell comprising a helper virus is contacted with an HSV-1 amplicon or HSV-1 hybrid amplicon described herein, resulting in the production of a virus comprising one or more recombinant nucleic acids described herein. In some embodiments, the virus is collected from the supernatant of the contacted host cell. Methods of generating virus by contacting host cells comprising a helper virus with an HSV-1 amplicon or HSV-1 hybrid amplicon are known in the art.

In some embodiments, the host cell is a complementing host cell. In some embodiments, the complementing host cell expresses one or more genes that are inactivated in any of the viral vectors described herein. In some embodiments, the complementing host cell is contacted with a recombinant herpes virus genome (e.g., a recombinant herpes simplex virus genome) described herein. In some embodiments, contacting a complementing host cell with a recombinant herpes virus genome results in the production of a herpes virus comprising one or more recombinant nucleic acids described herein. In some embodiments, the virus is collected from the supernatant of the contacted host cell. Methods of generating virus by contacting complementing host cells with a recombinant herpes simplex virus are generally described in WO2015/009952, WO2017/176336, WO2019/200163, and/or WO2019/210219.

### VIII. Articles of Manufacture or Kits

Certain aspects of the present disclosure relate to an article of manufacture or a kit comprising any of the recombinant nucleic acids, viruses, medicaments, and/or pharmaceutical compositions or formulations described herein. In some embodiments, the article of manufacture or kit comprises a package insert comprising instructions for administering the recombinant nucleic acid, virus, medicament, and/or pharmaceutical composition or formulation.

Suitable containers for the recombinant nucleic acids, viruses, medicaments, and/or pharmaceutical compositions or formulations may include, for example, bottles, vials, bags, tubes, and syringes. The container may be formed from a variety of materials such as glass, plastic (such as polyvinyl chloride or polyolefin), or metal alloy (such as stainless steel or hastelloy). In some embodiments, the container comprises a label on, or associated with the container, wherein the label indicates directions for use. The article of manufacture or kit may further include other materials desirable from a commercial and user standpoint, including other buffers, diluents, filters, inhalers, nebulizers, intranasal administration devices, a package insert, and the like.

### IX. Enumerated Embodiments

Embodiment 1: a recombinant herpes virus genome comprising one or more polynucleotides encoding a polypeptide.
Embodiment 2: the recombinant herpes virus genome of embodiment 1, wherein the recombinant herpes virus genome is replication competent.
Embodiment 3: the recombinant herpes virus genome of embodiment 1 or 2, wherein the recombinant herpes virus genome is replication defective.
Embodiment 4: the recombinant herpes virus genome of any of embodiments 1-3, wherein the recombinant herpes virus genome is selected from the group consisting of a recombinant herpes simplex virus genome, a recombinant varicella zoster virus genome, a recombinant human cytomegalovirus genome, a recombinant herpesvirus 6A genome, a recombinant herpesvirus 6B genome, a recombinant herpesvirus 7 genome, a recombinant Epstein-Barr virus genome, a recombinant Kaposi's sarcoma-associated herpesvirus genome, and any derivatives thereof.
Embodiment 5: the recombinant herpes virus genome of any of embodiments 1-4, wherein the recombinant herpes virus genome is a recombinant herpes simplex virus genome.
Embodiment 6: the recombinant herpes virus genome of any of embodiments 1-5, wherein the recombinant herpes simplex virus genome is a recombinant herpes simplex virus type 1 (HSV-1) genome, a recombinant herpes simplex virus type 2 (HSV-2) genome, or any derivatives thereof.
Embodiment 7: the recombinant herpes virus genome of any of embodiments 1-6, wherein the recombinant herpes simplex virus genome is a recombinant herpes simplex virus type 1 (HSV-1) genome.
Embodiment 8: the recombinant herpes virus genome of any of embodiments 1-7, wherein the recombinant herpes simplex virus genome is a recombinant herpes simplex virus type 2 (HSV-2) genome.
Embodiment 9: the recombinant herpes virus genome of any of embodiments 1-8, wherein the recombinant herpes simplex virus genome has been engineered to reduce or eliminate expression of one or more toxic herpes simplex virus genes.
Embodiment 10: the recombinant herpes virus genome of any of embodiments 1-9, wherein the recombinant herpes simplex virus genome comprises an inactivating mutation.
Embodiment 11: the recombinant herpes virus genome of any of embodiments 1-10, wherein the inactivating mutation is in a herpes simplex virus gene.
Embodiment 12: the recombinant herpes virus genome of any of embodiments 1-11, wherein the inactivating mutation is a deletion of the coding sequence of the herpes simplex virus gene.
Embodiment 13: the recombinant herpes virus genome of any of embodiments 1-12, wherein the herpes simplex virus gene is selected from the group consisting of Infected Cell Protein (ICP) 0, ICP4, ICP22, ICP27, ICP47, thymidine kinase (tk), Long Unique Region (UL) 41, and UL55.
Embodiment 14: the recombinant herpes virus genome of any of embodiments 1-13, wherein the recombinant herpes simplex virus genome comprises an inactivating mutation in one or both copies of the ICP4 gene.
Embodiment 15: the recombinant herpes virus genome of any of embodiments 1-14, wherein the recombinant herpes simplex virus genome comprises an inactivating mutation in the ICP22 gene.
Embodiment 16: the recombinant herpes virus genome of any of embodiments 1-15, wherein the recombinant herpes simplex virus genome comprises an inactivating mutation in the UL41 gene.
Embodiment 17: the recombinant herpes virus genome of any of embodiments 1-16, wherein the recombinant herpes simplex virus genome comprises an inactivating mutation in one or both copies of the ICP0 gene.
Embodiment 18: the recombinant herpes virus genome of any of embodiments 1-17, wherein the recombinant herpes simplex virus genome comprises an inactivating mutation in the ICP27 gene.
Embodiment 19: the recombinant herpes virus genome of any of embodiments 1-18, wherein the recombinant herpes simplex virus genome comprises an inactivating mutation in the UL55 gene.
Embodiment 20: the recombinant herpes virus genome of any of embodiments 1-19, wherein the recombinant herpes simplex virus genome does not comprise an inactivating mutation in the ICP47 gene.
Embodiment 21: the recombinant herpes virus genome of any of embodiments 1-20, wherein the recombinant herpes simplex virus does not comprise an inactivating mutation in one or both copies of the ICP34.5 gene.
Embodiment 22: the recombinant herpes virus genome of any of embodiments 1-21, wherein the recombinant herpes simplex virus genome comprises the one or more polynucleotides encoding the polypeptide within one or both of the ICP4 viral gene loci.
Embodiment 23: the recombinant herpes virus genome of any of embodiments 1-22, wherein the recombinant herpes simplex virus genome comprises the one or more polynucleotides encoding the polypeptide within the ICP22 viral gene locus.
Embodiment 24: the recombinant herpes virus genome of any of embodiments 1-23, wherein the recombinant herpes simplex virus genome comprises the one or more polynucleotides encoding the polypeptide within the UL41 viral gene locus.
Embodiment 25: the recombinant herpes virus genome of any of embodiments 1-24, wherein the recombinant herpes simplex virus genome comprises the one or more polynucleotides encoding the polypeptide within one or both of the ICP0 viral gene loci.
Embodiment 26: the recombinant herpes virus genome of any of embodiments 1-25, wherein the recombinant herpes simplex virus genome comprises the one or more polynucleotides encoding the polypeptide within the ICP27 viral gene locus.
Embodiment 27: the recombinant herpes virus genome of any of embodiments 1-26, wherein the recombinant herpes simplex virus genome comprises the one or more polynucleotides encoding the polypeptide within the UL55 viral gene locus.
Embodiment 28: the recombinant herpes virus genome of any of embodiments 1-27, wherein the polypeptide is a human polypeptide.
Embodiment 29: the recombinant herpes virus genome of any of embodiments 1-28, wherein the polypeptide is selected from the group consisting of sterile alpha motif domain-containing protein 11, nephrocystin-4, espin, nicotinamide/nicotinic acid mononucleotide adenylyltransferase 1, mitofusin-2, ER membrane protein complex subunit 1, phospholipase A2 group V, dehydrodolichyl diphosphate synthase complex subunit, palmitoyl-protein thioesterase 1, elongation of very long chain fatty acids protein 1, Protein O-linked-mannose beta-1,2-N-acetylglucosaminyltransferase 1, Retinoid isomerohydrolase, Retinal-specific phospholipid-transporting ATPase, Collagen alpha-1 (XI), Guanine nucleotide-binding protein G(t) subunit alpha-2, Chloride channel CLIC-like protein 1, DNA damage-regulated autophagy modulator protein 2, U4/U6 small nuclear ribonucleoprotein Prp3, Alpha-endosulfine, Semaphorin-4A, Cyclic AMP-dependent transcription factor ATF-6 alpha, Hemicentin-1, Complement factor H, Protein crumbs homolog 1, Adiponectin receptor protein 1, Protein RD3, Serine/threonine-protein kinase Nek2, Feline leukemia virus subgroup C receptor-related protein 1, Usherin, Serologically defined colon cancer antigen 8, Olfactory receptor 2W3, NBAS subunit of NRZ tethering complex, Cytosolic carboxypeptidase-like protein 5, Zinc finger protein 513, Intraflagellar transport protein 172 homolog, Photoreceptor cilium actin regulator, EGF-containing fibulin-like extracellular matrix protein 1/TLE family member 5, Protein FAM161A, WD repeat-containing and planar cell polarity effector protein fritz homolog, Centrosome-associated protein ALMS1, U5 small nuclear ribonucleoprotein 200 kDa helicase, Metal transporter CNNM4, Cyclic nucleotide-gated cation channel alpha-3, Nephrocystin-1, Tyrosine-protein kinase Mer, Bardet-Biedl syndrome 5 protein, Ceramide kinase-like protein, Neurogenic differentiation factor 1, Transmembrane protein 237, Inward rectifier potassium channel 13, S-arrestin, Secreted phosphoprotein 24, CCA tRNA nucleotidyltransferase 1, mitochondrial, Sodium bicarbonate cotransporter 3, Leucine zipper transcription factor-like protein 1, Guanine nucleotide-binding protein G(t) subunit alpha-1, Three-prime repair exonuclease 1, MAP kinase-activated protein kinase 3, Ataxin-7, Vitamin K-dependent protein S, ADP-ribosylation factor-like protein 6, Interphotoreceptor matrix proteoglycan 2, IQ calmodulin-binding motif-containing protein 1, Rhodopsin, Nephrocystin-3, Clarin-1, Probable cationic amino acid transporter/Solute carrier family 7 member 14, Choline-phosphate cytidylyltransferase A, Centrosomal protein of 19 kDa, Rod cGMP-specific 3′,5′-cyclic phosphodiesterase subunit beta, Wolframin, Homeobox protein HMX1, Ras-related protein Rab-28, Coiled-coil and C2 domain-containing protein 2A, Prominin-1, Adhesion G protein-coupled receptor A3, Death domain-containing protein 1, WD repeat-containing protein 19, cGMP-gated cation channel alpha-1, CDGSH iron-sulfur domain-containing protein 2, Microsomal triglyceride transfer protein large subunit, Leucine-rich repeat, immunoglobulin-like domain and transmembrane domain-containing protein 3, Bardet-Biedl syndrome 7 protein, Bardet-Biedl syndrome 12 protein, Major facilitator superfamily domain-containing protein 8, Serine/threonine-protein kinase PLK4, Lecithin retinol acyltransferase, Toll-like receptor 3, Cytochrome P450 4V2, Spliceosome-associated protein CWC27 homolog, Centrosomal protein POC5, Versican core protein, Adhesion G-protein coupled receptor V1, COUP transcription factor 1, Mitochondrial outer membrane protein SLC25A46, Catenin alpha-1, Histidine-tRNA ligase, cytoplasmic, Rod cGMP-specific 3′,5′-cyclic phosphodiesterase subunit alpha, Metabotropic glutamate receptor 6, Serine/threonine-protein kinase MAK, Complement C2, Complement factor B, Tubby-related protein 1, Guanylyl cyclase-activating protein 1, Guanylyl cyclase-activating protein 2, Peripherin-2, Interphotoreceptor matrix proteoglycan 1, Protein eyes shut homolog, Collagen alpha-1 (IX) chain, Regulating synaptic membrane exocytosis protein 1, Lebercilin, Elongation of very long chain fatty acids protein 4, PR domain zinc finger protein 13, Reticulon-4-interacting protein 1, mitochondrial, Jouberin, Peroxisomal targeting signal 2 receptor, CCR4-NOT transcription complex subunit 9, Aryl hydrocarbon receptor, Kelch-like protein 7, Retinitis pigmentosa 9 protein, Protein PTHB1, Peroxisomal ATPase PEX1, Tetraspanin-12, Inosine-5′-monophosphate dehydrogenase 1, Short-wave-sensitive opsin 1, UPF0606 protein KIAA1549, Retinitis pigmentosa 1-like 1 protein, Disintegrin and metalloproteinase domain-containing protein 9, Heparan-alpha-glucosaminide N-acetyltransferase, Oxygen-regulated protein 1, Alpha-tocopherol transfer protein, Centrosome and spindle pole-associated protein 1, Dynamin-like 120 kDa protein, mitochondrial, Peroxisome biogenesis factor 2, Cyclic nucleotide-gated cation channel beta-3, Cilia- and flagella-associated protein 418, Growth/differentiation factor 6, Regulating synaptic membrane exocytosis protein 2, Potassium voltage-gated channel subfamily V member 2, E3 ubiquitin-protein ligase Topors, Centrosomal protein of 78 kDa, Inversin, U4/U6 small nuclear ribonucleoprotein Prp4, Whirlin, E3 ubiquitin-protein ligase TRIM32, Toll-like receptor 4, Cytoplasmic dynein 2 intermediate chain 2, Programmed cell death protein 2, Exosome complex component RRP4, Phosphatidylinositol polyphosphate 5-phosphatase type IV, Phytanoyl-CoA dioxygenase, peroxisomal, Acyl-CoA-binding domain-containing protein, Protocadherin-15, Retinol-binding protein 3, DNA excision repair protein ERCC-6, Hexokinase-1, Cadherin-23, Cadherin-related family member 1, RPE-retinal G protein-coupled receptor, Kinesin-like protein KIF11, Retinol-binding protein 4, Cone cGMP-specific 3′,5′-cyclic phosphodiesterase subunit alpha, Paired box protein Pax-2, PDZ domain-containing protein 7, ADP-ribosylation factor-like protein 3, BBSome-interacting protein 1, Age-related maculopathy susceptibility protein 2, Serine protease HTRA1, Ornithine aminotransferase, mitochondrial, Zinc finger protein 408, Tubby protein homolog, Transcriptional enhancer factor TEF-1, Harmonin, Transmembrane protein 216, Bestrophin-1, Isoaspartyl peptidase/L-asparaginase, Rod outer segment membrane protein 1, Bardet-Biedl syndrome 1 protein, Calcium-binding protein 4, Low-density lipoprotein receptor-related protein 5, Calpain-5, Unconventional myosin-VIIa, Transmembrane protein 126A, Frizzled-4, Cytoplasmic dynein 2 light intermediate chain 1, Centrosomal protein of 164 kDa, Complement C1q tumor necrosis factor-related protein 5, Membrane frizzled-related protein, Voltage-dependent calcium channel subunit alpha-2/delta-4, Guanine nucleotide-binding protein G(I)/G(S)/G(T) subunit beta-3, Retinal cone rhodopsin-sensitive cGMP 3′,5′-cyclic phosphodiesterase subunit gamma, Collagen alpha-1 (II) chain, Matrix metalloproteinase-19, Retinol dehydrogenase 5, T-complex protein 1 subunit beta, Bardet-Biedl syndrome 10 protein, Centrosomal protein of 290 kDa, POC1 centriolar protein homolog B, Mevalonate kinase, Intraflagellar transport protein 81 homolog, Mitochondrial translation release factor in rescue, Integral membrane protein 2B, Retinoblastoma-associated protein, RCC1 and BTB domain-containing protein 1, Rhodopsin kinase GRK1, X-linked retinitis pigmentosa GTPase regulator-interacting protein 1, Neural retina-specific leucine zipper protein, Homeobox protein OTX2, Retinol dehydrogenase 11, Retinol dehydrogenase 12, Tubulin polyglutamylase TTLL5, Spermatogenesis-associated protein 7, Tetratricopeptide repeat protein 8, Fibulin-5, Transient receptor potential cation channel subfamily M member 1, Gamma-tubulin complex component 4, Sodium/potassium/calcium exchanger 1, Photoreceptor-specific nuclear receptor, Bardet-Biedl syndrome 4 protein, Calcium and integrin-binding family member 2, Retinaldehyde-binding protein 1, N-acetylglucosamine-1-phosphotransferase subunit gamma, Intraflagellar transport protein 140 homolog, Clusterin-associated protein 1, ATP-binding cassette sub-family C member 6, Ketimine reductase mu-crystallin, Battenin, Zinc finger protein 423, Protein fantom, Bardet-Biedl syndrome 2 protein, ADP-ribosylation factor-like protein 2-binding protein, Cyclic nucleotide-gated cation channel beta-1, Cadherin-3, Pre-mRNA-splicing factor ATP-dependent RNA helicase PRP16, A disintegrin and metalloproteinase with thrombospondin motifs 18, Solute carrier family 38 member 8, Retinal guanylyl cyclase 1, Pre-mRNA-processing-splicing factor 8, Aryl-hydrocarbon-interacting protein-like 1, Membrane-associated phosphatidylinositol transfer protein 3, Protein unc-119 homolog A, Probable G-protein coupled receptor 179, Tectonic-like complex member MKS1, Carbonic anhydrase 4, Regulator of G-protein signaling 9, Arylsulfatase G, pre-mRNA splicing regulator USH1G, Photoreceptor disk component PRCD, Fascin-2, Retinal rod rhodopsin-sensitive cGMP 3′,5′-cyclic phosphodiesterase subunit gamma, Laminin subunit alpha-1, AFG3-like protein 2, Cone-rod homeobox protein, Receptor expression-enhancing protein 6, Retina and anterior neural fold homeobox protein 2, Complement C3, Rho guanine nucleotide exchange factor 18, Patatin-like phospholipase domain-containing protein 6, Regulator of G-protein signaling 9-binding protein, Optic atrophy 3 protein, U4/U6 small nuclear ribonucleoprotein Prp31, Isocitrate dehydrogenase [NAD] subunit beta, mitochondrial, Pantothenate kinase 2, mitochondrial, Protein jagged-1, Molecular chaperone MKKS, Centrosomal protein kizuna, Lysophosphatidylserine lipase ABHD12, Kinesin-like protein KIF3B, Centrosome-associated protein CEP250, Pre-mRNA-processing factor 6, Cilia- and flagella-associated protein 410, Dynamin-1-like protein, Metalloproteinase inhibitor 3, Intraflagellar transport protein 27 homolog, Fibulin-1, MIEF1 upstream open reading frame protein, Aconitate hydratase, mitochondrial, Gamma-tubulin complex component 6, Centriole and centriolar satellite protein, Retinoschisin, Protein XRP2, Dystrophin, X-linked retinitis pigmentosa GTPase regulator, Nyctalopin, Xaa-Pro dipeptidase, Norrin, Voltage-dependent L-type calcium channel subunit alpha-1F, Phosphoglycerate kinase 1, Rab proteins geranylgeranyltransferase component A 1, Mitochondrial import inner membrane translocase subunit Tim8 A, Ribose-phosphate pyrophosphokinase 1, Long-wave-sensitive opsin 1, Medium-wave-sensitive opsin 1, Short-wave-sensitive opsin 1, Transcription factor A, mitochondrial, NADH-ubiquinone oxidoreductase chain 1, NADH-ubiquinone oxidoreductase chain 2, NADH-ubiquinone oxidoreductase chain 3, NADH-ubiquinone oxidoreductase chain 4L, NADH-ubiquinone oxidoreductase chain 4, NADH-ubiquinone oxidoreductase chain 5, NADH-ubiquinone oxidoreductase chain 6, ATP synthase subunit a, ATP synthase protein 8, Cytochrome c oxidase subunit 1, Cytochrome c oxidase subunit 3, Cytochrome b, Leucine-tRNA ligase, mitochondrial, Nondiscriminating glutamyl-tRNA synthetase EARS2, mitochondrial, Lysine-tRNA ligase, Histidine-tRNA ligase, mitochondrial, Serine-tRNA ligase, mitochondrial, Probable proline-tRNA ligase, mitochondrial, Cyanocobalamin reductase/alkylcobalamin dealkylase, POU domain, class 3, transcription factor 4, Ribosomal protein S6 kinase alpha-6, Ciliogenesis and planar polarity effector 1, Meckelin, TRAF3-interacting protein 1, Intraflagellar transport protein 74 homolog, S phase cyclin A-associated protein in the endoplasmic reticulum, Sodium channel and clathrin linker 1, Protein TALPID3, Tectonic-2, ADP-ribosylation factor-like protein 13B, B9 domain-containing protein 1, B9 domain-containing protein 2, C2 domain-containing protein 3, Centrosomal protein of 41 kDa, Centrosomal protein of 104 kDa, Centrosomal protein of 120 kDa, Intraflagellar transport protein 172 homolog, Katanin-interacting protein, Kinesin-like protein KIF7, Retinal rod rhodopsin-sensitive cGMP 3′,5′-cyclic phosphodiesterase subunit delta, Tectonic-1, Tectonic-3, Transmembrane protein 107, Transmembrane protein 138, Transmembrane protein 231, Tetratricopeptide repeat protein 21B, Nuclear receptor ROR-alpha, Beta-nerve growth factor, Collagen alpha-2 (VIII) chain, Solute carrier family 4 member 11, Zinc finger E-box-binding homeobox 1, Keratin, type II cuticular Hb3, Keratin, type I cytoskeletal 12, Transforming growth factor-beta-induced protein ig-h3, Tumor-associated calcium signal transducer 2, Carbohydrate sulfotransferase 6, Gelsolin, UbiA prenyltransferase domain-containing protein 1, Decorin, 1-phosphatidylinositol 3-phosphate 5-kinase, Transcription factor Ovo-like 2, and Grainyhead-like protein 2 homolog.
Embodiment 30: the recombinant herpes virus genome of any of embodiments 1-29, wherein the polypeptide comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence selected from the group consisting of SEQ ID NOS: 21-377 and SEQ ID NOs: 739-740.
Embodiment 31: the recombinant herpes virus genome of any of embodiments 1-30, wherein the polypeptide is beta-nerve growth factor, retinal-specific phospholipid-transporting ATPase, or centrosomal protein of 290 kDa.
Embodiment 32: the recombinant herpes virus genome of any of embodiments 1-31, wherein the polypeptide is beta-nerve growth factor.
Embodiment 33: the recombinant herpes virus genome of any of embodiments 1-32, wherein the beta-nerve growth factor comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 363, SEQ ID NO: 739, or SEQ ID NO: 740.
Embodiment 34: the recombinant herpes virus genome of any of embodiments 1-33, wherein the polypeptide is retinal-specific phospholipid-transporting ATPase.
Embodiment 35: the recombinant herpes virus genome of any of embodiments 1-34, wherein the retinal-specific phospholipid-transporting ATPase comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33.
Embodiment 36: the recombinant herpes virus genome of any of embodiments 1-35, wherein the polypeptide is centrosomal protein of 290 kDa.
Embodiment 37: the recombinant herpes virus genome of any of embodiments 1-36, wherein the centrosomal protein of 290 kDa comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 219.
Embodiment 38: the recombinant herpes virus genome of any of embodiments 1-37, wherein the recombinant herpes virus genome has reduced cytotoxicity when introduced into a target cell as compared to a corresponding wild-type herpes virus genome.
Embodiment 39: the recombinant herpes virus genome of any of embodiments 1-38, wherein the target cell is a human cell.
Embodiment 40: the recombinant herpes virus genome of any of embodiments 1-39, wherein the target cell is a cell of the eye.
Embodiment 41: the recombinant herpes virus genome of any of embodiments 1-40, wherein the target cell is a rod cell, a cone cell, a retinal ganglion cell, a bipolar cell, a horizontal cell, a muller cell, or an amacrine cell.
Embodiment 42: a herpes virus comprising the recombinant herpes virus genome of any one of embodiment 1-41.
Embodiment 43: the herpes virus of embodiment 42, wherein the herpes virus is replication competent.
Embodiment 44: the herpes virus of embodiment 42 or 43, wherein the herpes virus is replication defective.
Embodiment 45: the herpes virus of any of embodiments 42-44, wherein the herpes virus has reduced cytotoxicity as compared to a corresponding wild-type herpes virus.
Embodiment 46: the herpes virus of any of embodiments 42-45, wherein the herpes virus is selected from the group consisting of a herpes simplex virus, a varicella zoster virus, a human cytomegalovirus, a herpesvirus 6A, a herpesvirus 6B, a herpesvirus 7, an Epstein-Barr virus, and a Kaposi's sarcoma-associated herpesvirus.
Embodiment 47: the herpes virus of any of embodiments 42-46, wherein the herpes virus is a herpes simplex virus.
Embodiment 48: the herpes virus of any of embodiments 42-47, wherein the herpes simplex virus is a herpes simplex virus type 1 (HSV-1), a herpes simplex virus type 2 (HSV-2), or any derivatives thereof.
Embodiment 49: the herpes virus of any of embodiments 42-48, wherein the herpes simplex virus is a herpes simplex virus type 1 (HSV-1).
Embodiment 50: the herpes virus of any of embodiments 42-49, wherein the herpes simplex virus is a herpes simplex virus type 2 (HSV-2).
Embodiment 51: a pharmaceutical composition comprising the recombinant herpes virus genome of any of embodiments 1-41 or the herpes virus of any of embodiments 42-50 and a pharmaceutically acceptable excipient.
Embodiment 52: the pharmaceutical composition of embodiment 51, wherein the pharmaceutical composition is suitable for ocular, subretinal, intraocular, intravitreal, topical, subcutaneous, subconjunctival, subtenon, subtenon capsule, intracameral, retrobulbar, systemic, parenteral, periocular, juxtascleral, anterior juxtascleral, posterior juxtascleral, oral, peribulbar, or suprachoroidal administration.
Embodiment 53: the pharmaceutical composition of embodiment 51 or 52, wherein the pharmaceutical composition is suitable for intraocular, subretinal, intravitreal, suprachoroidal, or topical administration.
Embodiment 54: the pharmaceutical composition of any of embodiments 51-53, wherein the pharmaceutical composition is suitable for topical administration.
Embodiment 55: the pharmaceutical composition of any of embodiments 51-54, wherein the pharmaceutical composition is suitable for suprachoroidal administration.
Embodiment 56: the pharmaceutical composition of any of embodiments 51-55, wherein the pharmaceutical composition is suitable for use in an eyedrop, an ointment, a paste, a cream, a suspension, an emulsion, a fatty ointment, a gel, a bioadhesive gel, a powder, a lotion, a solution, a spray or any combinations thereof.
Embodiment 57: the pharmaceutical composition of any of embodiments 51-56, wherein the herpes virus or pharmaceutical composition is formulated as an eye drop, an ointment, a paste, a cream, a suspension, an emulsion, a fatty ointment, a gel, a bioadhesive gel, a powder, a lotion, a solution, or a spray.
Embodiment 58: the herpes virus of any of embodiments 42-50 or the pharmaceutical composition of any of embodiments 51-57 for use as a medicament.
Embodiment 59: the herpes virus of any of embodiments 42-50 or the pharmaceutical composition of any of embodiments 51-57 for use in a therapy.
Embodiment 60: the herpes virus of any of embodiments 42-50 or the pharmaceutical composition of any of embodiments 51-57, wherein the herpes virus or the pharmaceutical composition is for use in the treatment of an eye condition or disease.
Embodiment 61: the herpes virus of any of embodiments 42-50 or the pharmaceutical composition of any of embodiments 51-57, wherein the eye condition or disease is selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital night blindness, congenital stationary night blindness, severe congenital stationary night blindness, recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, and dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bucklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, calcific band keratopathy.
Embodiment 62: the herpes virus or the pharmaceutical composition of embodiment 61, wherein the eye condition or disease is neurotrophic keratitis, Stargardt disease, or Leber congenital amaurosis.
Embodiment 63: the herpes virus or the pharmaceutical composition of embodiment 61 or 62, wherein the eye condition or disease is neurotrophic keratitis.
Embodiment 64: the herpes virus or the pharmaceutical composition of any of embodiments 61-63, wherein the eye condition or disease is Stargardt disease.
Embodiment 65: the herpes virus or the pharmaceutical composition of any of embodiments 61-64, wherein the eye condition or disease is Leber congenital amaurosis.
Embodiment 66: use of the herpes virus of any of embodiments 42-50 or the pharmaceutical composition of any of embodiments 51-57 in the manufacture of a medicament for an eye condition or disease.
Embodiment 67: the use of embodiment 66, wherein the eye condition or disease is selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital night blindness, congenital stationary night blindness, severe congenital stationary night blindness, recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis Ill gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and calcific band keratopathy.
Embodiment 68: the use of embodiment 66 or 67, wherein the eye condition or disease is neurotrophic keratitis, Stargardt disease, or Leber congenital amaurosis.
Embodiment 69: the use of any of embodiments 66-68, wherein the eye condition or disease is neurotrophic keratitis.
Embodiment 70: the use of any of embodiments 66-69, wherein the eye condition or disease is Stargardt disease.
Embodiment 71: the use of any of embodiments 66-70, wherein the eye condition or disease is Leber congenital amaurosis.
Embodiment 72: a method of expressing, enhancing, increasing, augmenting, and/or supplementing the levels of a polypeptide in one or more cells of an eye of a subject, the method comprising administering to the subject an effective amount of the herpes virus of any of embodiments 42-50 or the pharmaceutical composition of any of embodiments 51-57.
Embodiment 73: the method of embodiment 72, wherein the one or more cells are one or more rod cells, cone cells, retinal ganglion cells, bipolar cells, horizontal cells, muller cells, or amacrine cell.
Embodiment 74: a method of providing prophylactic, palliative, or therapeutic relief of one or more signs or symptoms of an eye condition or disease in a subject in need thereof, the method comprising administering to the subject an effective amount of the herpes virus of any of embodiments 42-50 or the pharmaceutical composition of any of embodiments 51-57.
Embodiment 75: a method of treating an eye condition or disease in a subject in need thereof, the method comprising administering to the subject an effective amount of the herpes virus of any of embodiments 42-50 or the pharmaceutical composition of any of embodiments 51-57.
Embodiment 76: the method of embodiment 74 or 75, wherein the eye condition or disease is selected from the group consisting of retinitis pigmentosa, recessive retinitis pigmentosa, severe recessive retinitis pigmentosa, dominant retinitis pigmentosa, dominant retinitis pigmentosa with choroidal involvement, recessive retinitis pigmentosa with para-arteriolar preservation of the RPE (PPRPE), recessive retinitis pigmentosa with posterior column ataxia (PCARP), recessive retinitis pigmentosa with erythrocytic microcytosis, syndromic recessive retinitis pigmentosa, recessive syndromic disease with retinitis pigmentosa, non-syndromic recessive retinitis pigmentosa, non-syndromic dominant retinitis pigmentosa, syndromic dominant retinitis pigmentosa, juvenile recessive retinitis pigmentosa, recessive retinitis pigmentosa and mental retardation, recessive retinitis pigmentosa with macular degeneration, severe early-onset recessive retinitis pigmentosa, recessive retinitis pigmentosa and skeletal anomalies, recessive retinitis pigmentosa and skeletal abnormalities, dominant retinitis pigmentosa with sensorineural deafness, recessive retinitis pigmentosa with hearing loss, recessive retinitis pigmentosa with hearing loss and additional disabilities, recessive retinitis pigmentosa with vitreal alterations, early onset with macular coloboma recessive retinitis pigmentosa, X-linked retinitis pigmentosa, recessive X-linked retinitis pigmentosa, dominant X-linked retinitis pigmentosa, severe X-linked retinitis pigmentosa, X-linked retinitis pigmentosa with mental retardation, X-linked retinitis pigmentosa with myopathy, Bardet-Biedl like retinitis pigmentosa, Bardet-Biedl like recessive retinitis pigmentosa, digenic retinitis pigmentosa, digenic retinitis pigmentosa with retinal outer segment membrane protein 1, digenic retinitis pigmentosa with PRPH2, recessive retinitis pigmentosa with early macular involvement, recessive deafness without retinitis pigmentosa, recessive congenital deafness without retinitis pigmentosa, recessive retinitis pigmentosa and recessive ataxia, recessive retinitis pigmentosa and dominant ataxia, mitochondrial retinitis pigmentosa, mitochondrial retinitis pigmentosa with developmental and neurological abnormalities, mitochondrial retinitis pigmentosa with progressive sensorineural hearing loss, mitochondrial retinitis pigmentosa with deafness and neurological abnormalities, Autosomal Dominant Retinitis Pigmentosa, Nonsyndromic Autosomal Dominant Retinitis Pigmentosa, Autosomal Recessive, Retinitis Pigmentosa, Nonsyndromic Autosomal Recessive Retinitis Pigmentosa, Retinal pigment epithelium (RPE), recessive RPE, dominant RPE, recessive RPE degeneration, dominant RPE degeneration, ataxia, recessive ataxia, dominant ataxia, Senior-Loken syndrome, recessive Senior-Loken syndrome, dominant Senior-Loken syndrome, nephronophthisis, recessive nephronophthisis, dominant nephronophthisis, adolescent recessive nephronophthisis, ciliopathy-related recessive nephronophthisis, juvenile nephronophthisis, juvenile recessive nephronophthisis, juvenile dominant nephronophthisis, Usher syndrome, Usher syndrome type I, Usher syndrome type II, Usher syndrome type III, recessive Usher syndrome, atypical recessive Usher syndrome, dominant Usher syndrome, type 2 recessive Usher syndrome, type 2a recessive Usher syndrome, type 3 recessive Usher syndrome, type 3-like recessive Usher syndrome, atypical recessive Usher syndrome, type 1 recessive Usher syndrome, type 1b recessive Usher syndrome, type 1d recessive Usher syndrome, type 1f recessive Usher syndrome, type 1J recessive Usher syndrome, type 1k recessive Usher syndrome, digenic Usher syndrome with CDH23, digenic Usher syndrome with PCDH15, Acadian recessive Usher syndrome, recessive atypical Usher syndrome (USH3-like), Leber congenital amaurosis, recessive Leber congenital amaurosis, dominant Leber congenital amaurosis, de novo Leber congenital amaurosis, dominant Leber congenital amaurosis and pituitary dysfunction, recessive Leber congenital amaurosis with myopathy, recessive Leber congenital amaurosis with severe childhood retinal dystrophy, mitochondrial Leber hereditary optic neuropathy, Leber hereditary optic neuropathy, optic atrophy, recessive optic atrophy, dominant optic atrophy, optic atrophy with ataxia, recessive optic atrophy with ataxia, dominant optic atrophy with ataxia, recessive optic atrophy with ataxia and 3-methylglutaconic aciduria, dominant optic atrophy with cataract, ataxia and areflexia, Kjer type dominant optic atrophy, dominant optic atrophy with sensorineural hearing loss, recessive cerebellar degeneration with optic atrophy, optic atrophy with deadness-dystonia syndrome, X-linked optic atrophy with deadness-dystonia syndrome, retinal dystrophy, recessive retinal dystrophy, dominant retinal dystrophy, inherited retinal dystrophy, early onset recessive retinal dystrophy, recessive syndromic retinal dystrophy, recessive non-syndromic retinal dystrophy, recessive syndromic and non-syndromic retinal dystrophy, recessive optic atrophy and retinal dystrophy, recessive retinal dystrophy and obesity, recessive retinal dystrophy and cerebellar dysplasia, syndromic recessive optic atrophy and retinal dystrophy, dominant retinal dystrophy with iris coloboma, spectrum of ciliopathies including retinal dystrophy, recessive spectrum of ciliopathies including retinal dystrophy, dominant spectrum of ciliopathies including retinal dystrophy, dominant optic atrophy with neuropathy and myopathy, dominant optic atrophy with intellectual disability and developmental delay, syndromic optic atrophy, non-syndromic optic atrophy, recessive non-syndromic optic atrophy, dominant non-syndromic optic atrophy, recessive syndromic optic atrophy, dominant syndromic optic atrophy, Charcot-Marie-Tooth disease, recessive Charcot-Marie-Tooth disease, dominant Charcot-Marie-Tooth disease, benign fleck retina, recessive benign fleck retina, dominant benign fleck retina, syndromic retinopathy, recessive syndromic retinopathy, dominant syndromic retinopathy, Batten disease, recessive Batten disease, dominant Batten disease, recessive Batten disease (ceroid-lipofuscinosis, neuronal 3), juvenile recessive Batten disease (ceroid-lipofuscinosis, neuronal 3), neuronal ceroid lipofuscinosis, recessive neuronal ceroid lipofuscinosis, recessive neuronal ceroid lipofuscinosis 1, dominant neuronal ceroid lipofuscinosis, Stargardt disease, recessive Stargardt disease, dominant Stargardt disease, juvenile Stargardt disease, late onset Stargardt disease, Stargardt-like macular dystrophy, recessive Stargardt-like macular dystrophy, dominant Stargardt-like macular dystrophy, macular dystrophy, recessive macular dystrophy, early onset macular dystrophy, adult onset macular dystrophy, early onset recessive macular dystrophy, adult onset recessive macular dystrophy, early adult onset recessive macular dystrophy, juvenile with hypotrichosis recessive macular dystrophy, dominant macular dystrophy, late onset dominant macular dystrophy, dominant macular dystrophy with lens zonules, bull's-eye dominant macular dystrophy, butterfly-shaped dominant macular dystrophy, age-related dominant macular dystrophy, benign concentric annular dominant macular dystrophy, vitelliform recessive macular dystrophy, vitelliform dominant macular dystrophy, atypical vitelliform dominant macular dystrophy, dominant adult vitelliform macular dystrophy, Stargardt-like dominant macular dystrophy, Stargardt type dominant macular dystrophy, North Carolina dominant macular dystrophy, North Carolina-like dominant macular dystrophy, North Carolina-like with progressive sensorineural hearing loss dominant macular dystrophy, cystoid dominant macular dystrophy, Best type dominant macular dystrophy, occult macular dystrophy, recessive occult macular dystrophy, dominant occult macular dystrophy, age-related familial macular dystrophy, X-linked atrophic macular dystrophy, recessive X-linked atrophic macular dystrophy, dominant X-linked atrophic macular dystrophy, mitochondrial macular pattern dystrophy with type II diabetes and deafness, fundus flavimaculatus, recessive fundus flavimaculatus, dominant fundus flavimaculatus, rod dystrophy, recessive rod dystrophy, dominant rod dystrophy, cone dystrophy, recessive cone dystrophy, dominant cone dystrophy, recessive cone dystrophy with supernormal rod electroretinogram, early onset recessive cone dystrophy, late onset recessive cone dystrophy, cone dystrophy 1, X-linked cone dystrophy 1, cone dystrophy 2, progressive cone dystrophy 2, X-linked progressive cone dystrophy 2, delayed cone adaptation, recessive delayed cone adaptation, dominant delayed cone adaptation, cone-rod dystrophy, recessive cone-rod dystrophy, dominant cone-rod dystrophy, isolated cone-rod dystrophy, progressive cone-rod dystrophy, X-linked cone-rod dystrophy, X-linked progressive cone-rod dystrophy, progressive dominant cone-rod dystrophy, cone-rod dystrophy and amelogenesis imperfecta syndrome, recessive cone-rod dystrophy and amelogenesis imperfecta syndrome, dominant cone-rod dystrophy and amelogenesis imperfecta syndrome, recessive cone-rod dystrophy with inner retinopathy, recessive cone-rod dystrophy with skeletal disease, congenital syndromic nonprogressive recessive cone-rod dystrophy, recessive cone-rod dystrophy with hearing loss, recessive cone-rod dystrophy with psychomotor delay, recessive cone and cone-rod dystrophy, cone-rod synaptic disease, congenital cone-rod synaptic disease, recessive congenital cone-rod synaptic disease, dominant congenital cone-rod synaptic disease, rod-cone dystrophy, recessive rod-cone dystrophy, dominant rod-cone dystrophy, early onset recessive rod-cone dystrophy, non-syndromic recessive rod-cone dystrophy, recessive Newfoundland rod-cone dystrophy, recessive progressive cone dystrophy, Stickler syndrome, dominant Stickler syndrome, recessive Stickler syndrome, dominant Stickler syndrome type I, dominant Stickler syndrome type II, Marshall syndrome, dominant Marshall syndrome, recessive Marshall syndrome, achromatopsia, recessive achromatopsia, dominant achromatopsia, recessive complete achromatopsia, recessive incomplete achromatopsia, macular degeneration, age-related macular degeneration, complex etiology age-related macular degeneration, isolated age-related macular degeneration, wet age-related macular degeneration, dry age-related macular degeneration, drusen, recessive drusen, dominant drusen, early-onset recessive drusen, early-onset dominant drusen, macular drusen, dominant radial macular drusen, pigmented paravenous chorioretinal atrophy, recessive pigmented paravenous chorioretinal atrophy, dominant pigmented paravenous chorioretinal atrophy, progressive Bifocal Chorioretinal Atrophy, Bardet-Biedl syndrome, recessive Bardet-Biedl syndrome, dominant Bardet-Biedl syndrome, recessive Bardet-Biedl syndrome with developmental anomalies, retinal degeneration, recessive retinal degeneration, dominant retinal degeneration, non-syndromic recessive retinal degeneration, Doyne honeycomb retinal degeneration, recessive Doyne honeycomb retinal degeneration, dominant Doyne honeycomb retinal degeneration (Malattia Leventinese), recessive nephronophthisis with retinal degeneration, Alström syndrome, recessive Alström syndrome, dominant Alström syndrome, Joubert syndrome, recessive Joubert syndrome, dominant Joubert syndrome, Jobert syndrome, recessive Jobert syndrome, dominant Jobert syndrome, X-linked Jobert syndrome, vitreoretinal degeneration, recessive vitreoretinal degeneration, dominant vitreoretinal degeneration, snowflake vitreoretinal degeneration, snowflake dominant vitreoretinal degeneration, Oguchi disease, recessive Oguchi disease, dominant Oguchi disease, night blindness, stationary night blindness, congenital recessive congenital stationary night blindness, recessive complete congenital stationary night blindness, dominant congenital stationary night blindness, Nougaret type dominant congenital stationary night blindness, Oguchi type recessive congenital stationary night blindness, Riggs type recessive congenital stationary night blindness, Schubert-Bornschein type recessive congenital stationary night blindness, fundus albipunctatus type recessive congenital stationary night blindness, complete recessive congenital stationary night blindness, X-linked congenital stationary night blindness, incomplete X-linked congenital stationary night blindness, retinal vasculopathy, cerebral leukodystrophy, retinal vasculopathy with cerebral leukodystrophy, recessive retinal vasculopathy with cerebral leukodystrophy, dominant retinal vasculopathy with cerebral leukodystrophy, Aicardi-Goutiere syndrome, Aicardi-Goutiere syndrome 1, recessive Aicardi-Goutiere syndrome 1, dominant Aicardi-utiere syndrome 1, chilblain lupus, recessive chilblain lupus, dominant chilblain lupus, Martinique retinal dystrophy and retinitis pigmentosa, recessive Martinique retinal dystrophy and retinitis pigmentosa, dominant Martinique retinal dystrophy and retinitis pigmentosa, spinocerebellar ataxia, recessive spinocerebellar ataxia, dominant spinocerebellar ataxia, spinocerebellar ataxia with macular dystrophy, spinocerebellar ataxia with retinal degeneration, spinocerebellar ataxia with macular dystrophy or retinal degeneration, recessive spinocerebellar ataxia with macular dystrophy or retinal degeneration, dominant spinocerebellar ataxia with macular dystrophy or retinal degeneration, Wolfram syndrome, recessive Wolfram syndrome, dominant Wolfram syndrome, low frequency sensorineural hearing loss, recessive low frequency sensorineural hearing loss, dominant low frequency sensorineural hearing loss, oculoauricular syndrome, recessive oculoauricular syndrome, dominant oculoauricular syndrome, recessive renal, skeletal and retinal anomalies, recessive abetalipoproteinemia, microcephaly, recessive microcephaly, dominant microcephaly, growth failure and retinopathy recessive microcephaly, Bietti crystalline corneoretinal dystrophy, recessive Bietti crystalline corneoretinal dystrophy, dominant Bietti crystalline corneoretinal dystrophy, Wagner disease, erosive vitreoretinopathy, Wagner disease and erosive vitreoretinopathy, recessive Wagner disease and erosive vitreoretinopathy, dominant Wagner disease and erosive vitreoretinopathy, febrile convulsions, recessive febrile convulsions, dominant febrile convulsions, dominant/recessive febrile convulsions, choroidal dystrophy, recessive choroidal dystrophy, dominant choroidal dystrophy, central areolar choroidal dystrophy, dominant central areolar choroidal dystrophy, recessive central areolar choroidal dystrophy, epiphyseal dysplasia, recessive epiphyseal dysplasia, dominant epiphyseal dysplasia, multiple epiphyseal dysplasia, recessive multiple epiphyseal dysplasia, dominant multiple epiphyseal dysplasia, ichthyosis, recessive ichthyosis, dominant ichthyosis, quadriplegia and retardation recessive ichthyosis, chorioretinal atrophy, bifocal chorioretinal atrophy, progressive bifocal chorioretinal atrophy, recessive progressive bifocal chorioretinal atrophy, dominant progressive bifocal chorioretinal atrophy, Refsum disease, recessive Refsum disease, dominant Refsum disease, adult form recessive Refsum disease, infantile form recessive Refsum disease, retinal-cone dystrophy, recessive retinal-cone dystrophy, dominant retinal-cone dystrophy, retinal-cone dystrophy 1, recessive retinal-cone dystrophy 1, dominant retinal-cone dystrophy 1, tritanopia, recessive tritanopia, dominant tritanopia, mucopolysaccharidosis, recessive mucopolysaccharidosis, dominant mucopolysaccharidosis, achromatopsia Pingelapese, recessive achromatopsia Pingelapese, dominant achromatopsia Pingelapese, Klippel-Feil syndrome, recessive Klippel-Feil syndrome, dominant Klippel-Feil syndrome, microphthalmia, recessive microphthalmia, dominant microphthalmia, limb-girdle muscular dystrophy, recessive limb-girdle muscular dystrophy, dominant limb-girdle muscular dystrophy, short-rib thoracic dysplasia, recessive short-rib thoracic dysplasia, dominant short-rib thoracic dysplasia, polydactyly recessive short-rib thoracic dysplasia, retinal dystrophy recessive short-rib thoracic dysplasia, mental retardation, truncal obesity, retinal dystrophy, and micropenis (MORM) syndrome, recessive MORM syndrome, dominant MORM syndrome, spasticity and retinal degeneration recessive retardation, Cockayne syndrome, recessive Cockayne syndrome, dominant Cockayne syndrome, congenital retinal nonattachment, recessive nonsyndromal congenital retinal nonattachment, dominant nonsyndromal congenital retinal nonattachment, hemolytic anemia, nonspherocytic hemolytic anemia, recessive nonspherocytic hemolytic anemia, dominant nonspherocytic hemolytic anemia, hereditary neuropathy, hereditary neuropathy (Russe type), recessive hereditary neuropathy (Russe type), dominant hereditary neuropathy (Russe type), choroidal sclerosis, recessive choroidal sclerosis, dominant choroidal sclerosis, retinopathy, recessive retinopathy, dominant retinopathy, combined dominant and recessive retinopathy, diffuse dominant retinopathy, variable dominant retinopathy, diffuse and variable dominant retinopathy, microcephaly, recessive microcephaly, dominant microcephaly, lymphedema dominant microcephaly, chorioretinopathy dominant microcephaly, lymphedema and chorioretinopathy dominant microcephaly, chorioretinopathy, recessive chorioretinopathy, dominant chorioretinopathy, chorioretinopathy and microcephaly, recessive chorioretinopathy and microcephaly, dominant chorioretinopathy and microcephaly, renal-coloboma syndrome, recessive renal-coloboma syndrome, dominant renal-coloboma syndrome, non-syndromic deafness, recessive non-syndromic deafness, dominant non-syndromic deafness, gyrate atrophy, recessive gyrate atrophy, dominant gyrate atrophy, gyrate atrophy of the choroid and retina, vitreoretinopathy, exudative vitreoretinopathy, familial exudative vitreoretinopathy, recessive familial exudative vitreoretinopathy, dominant familial exudative vitreoretinopathy, dominant familial exudative vitreoretinopathy and Coats disease, neovascular inflammatory vitreoretinopathy, recessive neovascular inflammatory vitreoretinopathy, dominant neovascular inflammatory vitreoretinopathy, atrophia areata, recessive atrophia areata, dominant atrophia areata, Meckel syndrome, recessive Meckel syndrome, dominant Meckel syndrome, vitreoretinochoroidopathy, recessive vitreoretinochoroidopathy, dominant vitreoretinochoroidopathy, bestrophinopathy, recessive bestrophinopathy, dominant bestrophinopathy, high bone mass trait, recessive high bone mass trait, dominant high bone mass trait, osteoporosis-pseudoglioma syndrome, recessive osteoporosis-pseudoglioma syndrome, dominant osteoporosis-pseudoglioma syndrome, microphthalmos, recessive microphthalmos, dominant microphthalmos, retinal disease syndrome, recessive retinal disease syndrome, dominant retinal disease syndrome, microphthalmos and retinal disease syndrome, recessive microphthalmos and retinal disease syndrome, dominant microphthalmos and retinal disease syndrome, nanophthalmos, recessive nanophthalmos, dominant nanophthalmos, bone dysplasias, recessive bone dysplasias, dominant bone dysplasias, developmental disorders, osteoarthritic diseases and syndromic disorders, cavitary optic disc anomalies, recessive cavitary optic disc anomalies, dominant cavitary optic disc anomalies, fundus albipunctatus, recessive fundus albipunctatus, dominant fundus albipunctatus, mevalonic aciduria, recessive mevalonic aciduria, dominant mevalonic aciduria, hyper-IgD syndrome, recessive hyper-IgD syndrome, dominant hyper-IgD syndrome, spastic paraplegia, recessive spastic paraplegia, dominant spastic paraplegia, neuropathy recessive spastic paraplegia, optic atrophy recessive spastic paraplegia, neuropathy and optic atrophy recessive spastic paraplegia (dementia), recessive dementia, dominant dementia, familial dominant dementia, retinoblastoma, recessive retinoblastoma, dominant retinoblastoma, germline retinoblastoma, somatic retinoblastoma, dominant germline or somatic retinoblastoma, retinoma, benign retinoma, pinealoma, osteogenic sarcoma, rod monochromacy, recessive rod monochromacy, dominant rod monochromacy, achromatopsia, recessive achromatopsia, dominant achromatopsia, rod achromatopsia, rod recessive achromatopsia, rod dominant achromatopsia, recessive rod monochromacy or achromatopsia, pattern dystrophy, recessive pattern dystrophy, dominant pattern dystrophy, S-cone syndrome, enhanced S-cone syndrome (ESC), recessive ESC, dominant ESC, Goldmann-Favre syndrome, recessive Goldmann-Favre syndrome, dominant Goldmann-Favre syndrome, Bothnia dystrophy, recessive Bothnia dystrophy, dominant Bothnia dystrophy, retinitis punctata albescens, recessive retinitis punctata albescens, dominant retinitis punctata albescens, mucolipidosis III gamma, recessive mucolipidosis III gamma, dominant mucolipidosis III gamma, Mainzer-Saldino syndrome, recessive Mainzer-Saldino syndrome, dominant Mainzer-Saldino syndrome, pseudoxanthoma elasticum, recessive pseudoxanthoma elasticum, dominant pseudoxanthoma elasticum, Knobloch syndrome, recessive Knobloch syndrome, dominant Knobloch syndrome, foveal hypoplasia, recessive foveal hypoplasia, dominant foveal hypoplasia, anterior segment dysgenesis, recessive anterior segment dysgenesis, dominant anterior segment dysgenesis, foveal hypoplasia and anterior segment dysgenesis, recessive foveal hypoplasia and anterior segment dysgenesis, dominant foveal hypoplasia and anterior segment dysgenesis, spastic ataxia, recessive spastic ataxia, dominant spastic ataxia, de Grouchy syndrome, Boucher-Neuhauser syndrome, recessive Boucher-Neuhauser syndrome, dominant Boucher-Neuhauser syndrome, Boucher-Neuhauser syndrome with chorioretinal dystrophy, recessive Boucher-Neuhauser syndrome with chorioretinal dystrophy, dominant Boucher-Neuhauser syndrome with chorioretinal dystrophy, hypoprebetalipoproteinemia, acanthocytosis, retinitis pigmentosa, and palladial (HARP), degeneration, recessive HARP degeneration, dominant HARP degeneration, Hallervorden-Spatz syndrome, recessive Hallervorden-Spatz syndrome, dominant Hallervorden-Spatz syndrome, Alagille syndrome, recessive Alagille syndrome, dominant Alagille syndrome, polyneuropathy, hearing loss, ataxia, retinitis pigmentosa and early-onset cataract (PHARC), recessive PHARC, dominant PHARC, recessive syndromic PHARC, Sorsby's fundus dystrophy, recessive Sorsby's fundus dystrophy, dominant Sorsby's fundus dystrophy, vitreoretinal dystrophy, recessive vitreoretinal dystrophy, dominant vitreoretinal dystrophy, optic neuropathy, recessive optic neuropathy, dominant optic neuropathy, late onset dominant optic neuropathy, orofaciodigital syndrome 1, Simpson-Golabi-Behmel syndrome 2, X-linked retinoschisis, X-linked Oregon eye disease, X-linked optic atrophy, retinal dysplasia, recessive retinal dysplasia, dominant retinal dysplasia, X-linked retinal dysplasia, primary X-linked retinal dysplasia, X-linked Norrie disease, Coats disease, X-linked Åland Island eye disease, Autoimmune Inner Ear Disease (AIED), AIED-like disease, choroideremia, X-linked choroideremia, neuropathy, optic atrophy, deafness and retinitis pigmentosa, X-linked neuropathy, optic atrophy, deafness and retinitis pigmentosa, X-linked blue cone monochromacy, X-linked protanomaly and rare macular dystrophy in blue cone monochromacy (BCM), X-linked deuteranomaly and rare macular dystrophy in blue cone monochromacy (BCM), mitochondrial Kearns-Sayre syndrome including retinal pigmentary degeneration, Leigh syndrome, retinopathy, mitochondrial pigmentary retinopathy and sensorineural hearing loss, ocular albinism, oculocutaneous albinism, Neuronal ceroid lipofuscinoses, Zellweger spectrum disorder, Cobalamin C deficiency, blue-cone monochromatism, hereditary red-green color vision defects, tritan and yellow-blue defects, bradyopsia, delayed cone adaptation, Uveitis, Diabetic Retinopathy, Diabetic Macular Edema, Persistent Corneal Epithelial Defect, Neurotrophic Keratitis, Herpes Stromal Keratitis, Chronic Dry Eye, Glaucoma, ocular abrasion, ocular blistering, ocular scarring, vision loss, blindness, corneal blindness, dry eye disease, aqueous deficiency dry eye disease, Sjögren's syndrome-related aqueous deficiency dry eye disease, non-Sjögren's syndrome-related dry eye disease, evaporative dry eye disease, meibomian gland dysfunction, blepharitis, lid margin inflammation, ocular rosacea and atopy, aqueous deficiency and evaporative dry eye disease, Fuchs dystrophy, Fuchs endothelial corneal dystrophy (FECD), Fuchs endothelial dystrophy (FED), panuveitis, diffuse uveitis, total uveitis, cataract, partial cataracts, complete cataracts, stationary cataracts, progressive cataracts, hard cataracts, soft cataracts, nuclear cataracts, nuclear sclerosis cataracts, cortical cataracts, posterior subcapsular cataracts, congenital cataracts, corneal dystrophy, Epithelial and subepithelial dystrophies, Epithelial basement membrane dystrophy, Epithelial recurrent erosion dystrophies (Franceschetti corneal dystrophy, Dystrophia Smolandiensis, and/or Dystrophia Helsinglandica), Subepithelial mucinous corneal dystrophy, Meesmann corneal dystrophy, Lisch epithelial corneal dystrophy, Gelatinous drop-like corneal dystrophy, Bowman Layer dystrophies, Reis-Bücklers corneal dystrophy, Thiel-Behnke corneal dystrophy, Stromal dystrophies-TGFB1 corneal dystrophies, Lattice corneal dystrophy, type 1 variants (III, IIIA, I/IIIA, IV) of lattice corneal dystrophy, Granular corneal dystrophy, type 1, Granular corneal dystrophy, type 2, Stromal dystrophies, Macular corneal dystrophy, Schnyder crystalline corneal dystrophy, Congenital stromal corneal dystrophy, Fleck corneal dystrophy, Posterior amorphous corneal dystrophy, Central cloudy dystrophy of François, Pre-Descemet corneal dystrophy, Endothelial dystrophies, Fuchs' dystrophy, Posterior polymorphous corneal dystrophy, Congenital hereditary endothelial dystrophy, X-linked endothelial corneal dystrophy, Superficial dystrophies, Epithelial basement membrane dystrophy, Meesmann juvenile epithelial corneal dystrophy, Gelatinous drop-like corneal dystrophy, Lisch epithelial corneal dystrophy, Subepithelial mucinous corneal dystrophy, Reis-Bucklers corneal dystrophy, Thiel-Behnke dystrophy, Stromal dystrophies, Lattice corneal dystrophy, Granular corneal dystrophy, Macular corneal dystrophy, Schnyder crystalline corneal dystrophy, Congenital stromal corneal dystrophy, Fleck corneal dystrophy, Posterior dystrophies, Fuchs' dystrophy, Posterior polymorphous corneal dystrophy, Congenital hereditary endothelial dystrophy, keratopathy, band keratopathy, corneal band keratopathy, and calcific band keratopathy.
Embodiment 77: the method of any of embodiments 74-76, wherein the eye condition or disease is neurotrophic keratitis, Stargardt disease, or Leber congenital amaurosis.
Embodiment 78: the method of any of embodiments 74-77, wherein the eye condition or disease is neurotrophic keratitis.
Embodiment 79: the method of any of embodiments 74-78, wherein the eye condition or disease is Stargardt disease.
Embodiment 80: the method of any of embodiments 74-79, wherein the eye condition or disease is Leber congenital amaurosis.
Embodiment 81: the method of any of embodiments 72-80, wherein the subject is a human.
Embodiment 82: the method of any of embodiments 72-81, wherein the herpes virus or pharmaceutical composition is administered via injection, via injection to the eye, via subretinal injection, via intraocular injection, via intravitreal injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally to the subject.
Embodiment 83: the method of any of embodiments 72-82, wherein the herpes virus or pharmaceutical composition is administered via injection to the eye, via subretinal injection, via intravitreal injection, suprachoroidally, or topically to the subject.
Embodiment 84: the method of any of embodiments 72-83, wherein the herpes virus or pharmaceutical composition is administered topically to the subject.
Embodiment 85: the method of any of embodiments 72-84, wherein the herpes virus or pharmaceutical composition is administered via suprachoroidally to the subject.

The specification is considered to be sufficient to enable one skilled in the art to practice the present disclosure. Various modifications of the present disclosure in addition to those shown and described herein will become apparent to those skilled in the art from the foregoing description and fall within the scope of the appended claims.

### EXAMPLES

The present disclosure will be more fully understood by reference to the following examples. It should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Modified Herpes Simplex Virus Vectors Encoding a Polypeptide

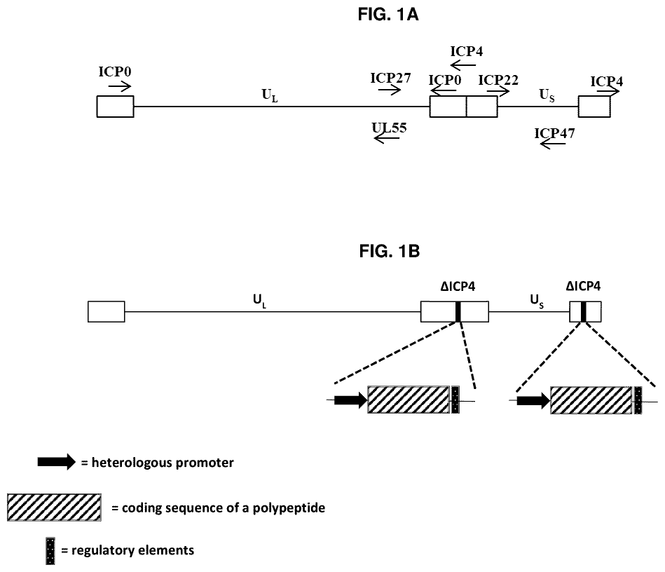

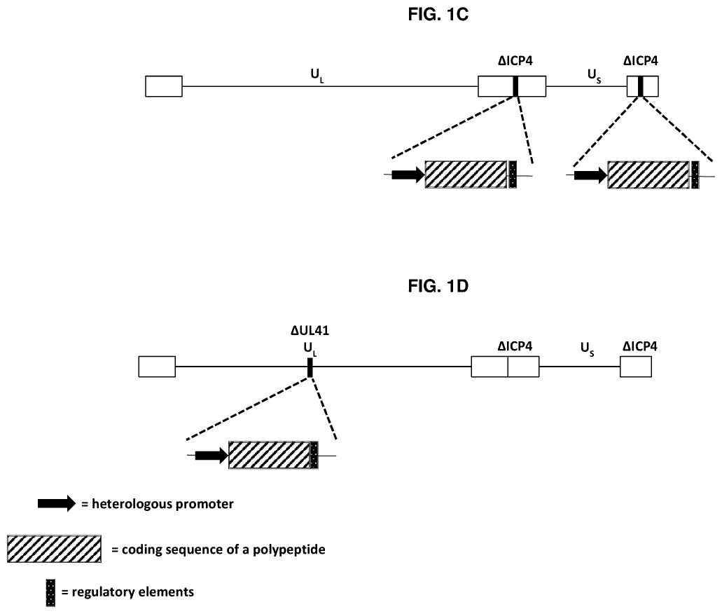

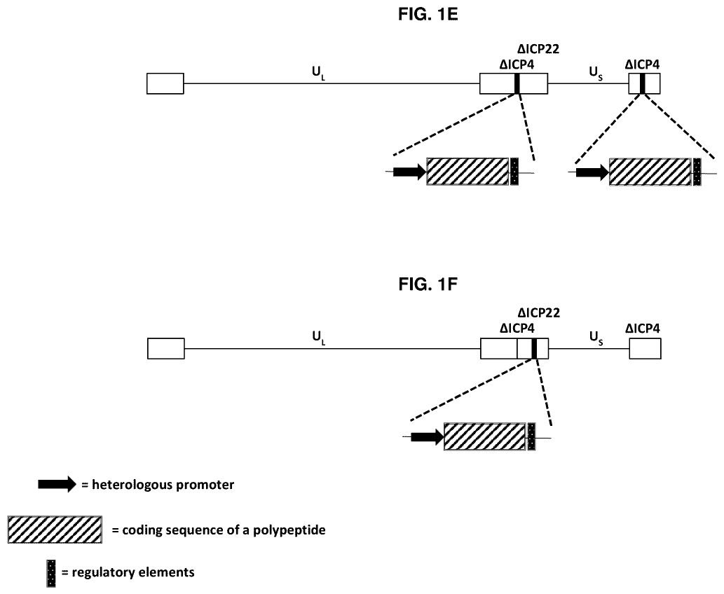

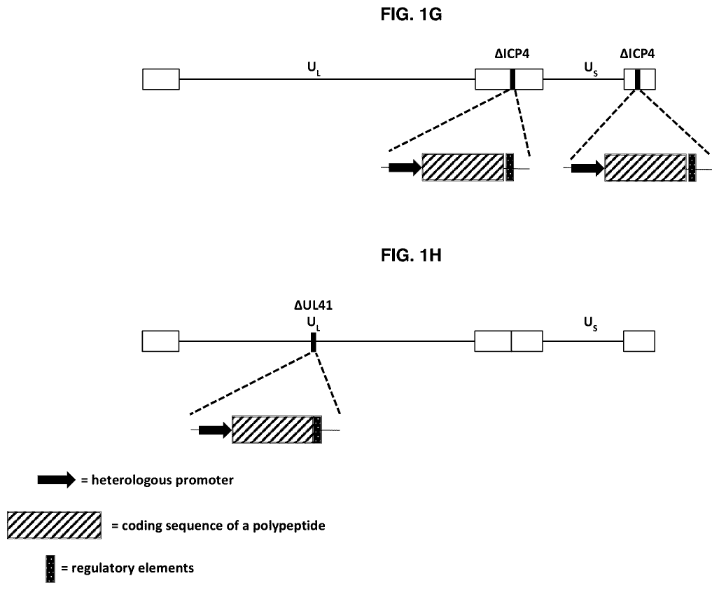

To make modified herpes simplex virus genome vectors capable of expressing polypeptides in a target mammalian cell (such as cells of the eye), a herpes simplex virus genome (FIG. 1A) is first modified to inactivate one or more herpes simplex virus genes. Such modifications may decrease the toxicity of the genome in mammalian cells. Next, variants of these modified/attenuated recombinant viral constructs are generated such that they carry one or more polynucleotides encoding the desired polypeptide. These variants include: a recombinant ΔICP4-modified HSV-1 genome comprising expression cassettes containing the coding sequence of a polypeptide under the control of a heterologous promoter integrated at each ICP4 locus (FIG. 1B); a recombinant ΔICP4/ΔUL41-modified HSV-1 genome comprising expression cassettes containing the coding sequence of a polypeptide under the control of a heterologous promoter integrated at each ICP4 locus (FIG. 1C); a recombinant ΔICP4/ΔUL41-modified HSV-1 genome comprising an expression cassette containing the coding sequence of a polypeptide under the control of a heterologous promoter integrated at the UL41 locus (FIG. 1D); a recombinant ΔICP4/ΔICP22-modified HSV-1 genome comprising expression cassettes containing the coding sequence of a polypeptide under the control of a heterologous promoter integrated at each ICP4 locus (FIG. 1E); a recombinant ΔICP4/ΔICP22-modified HSV-1 genome comprising an expression cassette containing the coding sequence of a polypeptide under the control of a heterologous promoter integrated at the ICP22 locus (FIG. 1F); a recombinant ΔICP4/ΔUL41/ΔICP22-modified HSV-1 genome comprising expression cassettes containing the coding sequence of a polypeptide under the control of a heterologous promoter integrated at each ICP4 locus (FIG. 1G); a recombinant ΔICP4/ΔUL41/ΔICP22-modified HSV-1 genome comprising an expression cassette containing the coding sequence of a polypeptide (e.g., SEQ ID NO: 3) under the control of a heterologous promoter integrated at the UL41 locus (FIG. 1H); and a recombinant ΔICP4/ΔUL41/ΔICP22-modified HSV-1 genome comprising an expression cassette containing the coding sequence of a polypeptide under the control of a heterologous promoter integrated at the ICP22 locus (FIG. 1I).

These modified herpes simplex virus genome vectors are transfected into engineered cells that are modified to express one or more herpes simplex virus genes. These engineered cells secrete into the supernatant of the cell culture a replication-defective herpes simplex virus with the modified genomes packaged therein. The supernatant is then collected, concentrated, and sterile filtered.

Modified herpes simplex virus genome vectors described herein can express any one or more of the exemplary polypeptides in Table 1 below, in any suitable combination.

TABLE 1
Representative Polypeptides and Polynucleotides

| | | UniProt | | | NCBI |
|---|---|---|---|---|---|
| Amino acid | | Accession | Nucleic acid | | Gene |
| SEQ ID NO. | Protein Name | No. | SEQ ID NO. | Gene Name | ID No. |
| 21 | Sterile alpha motif domain-containing protein 11 | Q96NU1 | 378 | SAMD11 | 148398 |
| 22 | Nephrocystin-4 | O75161 | 379 | NPHP4 | 261734 |
| 23 | Espin | B1AK53 | 380 | ESPN | 83715 |
| 24 | Nicotinamide/nicotinic acid mononucleotide | Q9HAN9 | 381 | NMNAT1 | 64802 |
| | adenylyltransferase 1 | | | | |
| 25 | Mitofusin-2 | O95140 | 382 | MFN2 | 9927 |
| 26 | ER membrane protein complex subunit 1 | Q8N766 | 383 | EMC1 | 23065 |
| 27 | Phospholipase A2 group V | P39877 | 384 | PLA2G5 | 5322 |
| 28 | Dehydrodolichyl diphosphate synthase complex | Q86SQ9 | 385 | DHDDS | 79947 |
| | subunit | | | | |
| 29 | Palmitoyl-protein thioesterase 1 | P50897 | 386 | PPT1 | 5538 |
| 30 | Elongation of very long chain fatty acids protein 1 | Q9BW60 | 387 | ELOVL1 | 64834 |
| 31 | Protein O-linked-mannose beta-1,2-N- | Q8WZA1 | 388 | POMGNT1 | 55624 |
| | acetylglucosaminyltransferase 1 | | | | |
| 32 | Retinoid isomerohydrolase | Q16518 | 389 | RPE65 | 6121 |
| 33 | Retinal-specific phospholipid-transporting ATPase | P78363 | 390 | ABCA4 | 24 |
| 34 | Collagen alpha-1(XI) | P12107 | 391 | COL11A1 | 1301 |
| 35 | Guanine nucleotide-binding protein G(t) subunit | P19087 | 392 | GNAT2 | 2780 |
| | alpha-2 | | | | |
| 36 | Chloride channel CLIC-like protein 1 | Q96S66 | 393 | CLCC1 | 23155 |
| 37 | DNA damage-regulated autophagy modulator | Q6UX65 | 394 | DRAM2 | 128338 |
| | protein 2 | | | | |
| 38 | U4/U6 small nuclear ribonucleoprotein Prp3 | O43395 | 395 | PRPF3 | 9129 |
| 39 | Alpha-endosulfine | O43768 | 396 | ENSA | 2029 |
| 40 | Semaphorin-4A | Q9H3S1 | 397 | SEMA4A | 64218 |
| 41 | Cyclic AMP-dependent transcription factor ATF-6 | P18850 | 398 | ATF6 | 22926 |
| | alpha | | | | |
| 42 | Hemicentin-1 | Q96RW7 | 399 | HMCN1 | 83872 |
| 43 | Complement factor H | P08603 | 400 | CFH | 3075 |
| 44 | Protein crumbs homolog 1 | P82279 | 401 | CRB1 | 23418 |
| 45 | Adiponectin receptor protein 1 | Q96A54 | 402 | ADIPOR1 | 51094 |
| 46 | Protein RD3 | Q7Z3Z2 | 403 | RD3 | 343035 |
| 47 | Serine/threonine-protein kinase Nek2 | P51955 | 404 | NEK2 | 4751 |
| 48 | Feline leukemia virus subgroup C receptor-related | Q9YSY0 | 405 | FLVCR1 | 28982 |
| | protein 1 | | | | |
| 49 | Usherin | O75445 | 406 | USH2A | 7399 |
| 50 | Serologically defined colon cancer antigen 8 | Q86SQ7 | 407 | SDCCAG8 | 10806 |
| 51 | Olfactory receptor 2W3 | Q7Z3T1 | 408 | OR2W3 | 343171 |
| 52 | NBAS subunit of NRZ tethering complex | A2RRP1 | 409 | NBAS | 51594 |
| 53 | Cytosolic carboxypeptidase-like protein 5 | Q8NDL9 | 410 | AGBL5 | 60509 |
| 54 | Zinc finger protein 513 | Q8N8E2 | 411 | ZNF513 | 130557 |
| 55 | Intraflagellar transport protein 172 homolog | Q9UG01 | 412 | IFT172 | 26160 |
| 56 | Photoreceptor cilium actin regulator | A6NGG8 | 413 | PCARE | 388939 |
| 57 | EGF-containing fibulin-like extracellular matrix | Q12808 | 414 | EFEMP1/ | 2202 |
| | protein 1/TLE family member 5 | | | TLES | |
| 58 | Protein FAM161A | Q3B820 | 415 | FAM161A | 84140 |
| 59 | WD repeat-containing and planar cell polarity | O95876 | 416 | WDPCP | 51057 |
| | effector protein fritz homolog | | | | |
| 60 | Centrosome-associated protein ALMS1 | Q8TCU4 | 417 | ALMS1 | 7840 |
| 61 | US small nuclear ribonucleoprotein 200 kDa | O75643 | 418 | SNRNP200 | 23020 |
| | helicase | | | | |
| 62 | Metal transporter CNNM4 | Q6P4Q7 | 419 | CNNM4 | 26504 |
| 63 | Cyclic nucleotide-gated cation channel alpha-3 | Q16281 | 420 | CNGA3 | 1261 |
| 64 | Nephrocystin-1 | O15259 | 421 | NPHP1 | 4867 |
| 65 | Tyrosine-protein kinase Mer | Q12866 | 422 | MERTK | 10461 |
| 66 | Bardet-Biedl syndrome 5 protein | Q8N3I7 | 423 | BBS5 | 129880 |
| 67 | Ceramide kinase-like protein | Q49MI3 | 424 | CERKL | 375298 |
| 68 | Neurogenic differentiation factor 1 | Q13562 | 425 | NEUROD1 | 4760 |
| 69 | Transmembrane protein 237 | Q96Q45 | 426 | TMEM237 | 65062 |
| 70 | Inward rectifier potassium channel 13 | O60928 | 427 | KCNJ13 | 3769 |
| 71 | S-arrestin | P10523 | 428 | SAG | 6295 |
| 72 | Secreted phosphoprotein 24 | Q13103 | 429 | SPP2 | 6694 |
| 73 | CCA IRNA nucleotidyltransferase 1, mitochondrial | Q96Q11 | 430 | TRNT1 | 51095 |
| 74 | Sodium bicarbonate cotransporter 3 | Q9Y6M7 | 431 | SLC4A7 | 9497 |
| 75 | Leucine zipper transcription factor-like protein 1 | Q9NQ48 | 432 | LZTFL1 | 54585 |
| 76 | Guanine nucleotide-binding protein G(t) subunit | P11488 | 433 | GNAT1 | 2779 |
| | alpha-1 | | | | |
| 77 | Three-prime repair exonuclease 1 | Q9NSU2 | 434 | TREX1 | 11277 |
| 78 | MAP kinase-activated protein kinase 3 | Q16644 | 435 | MAPKAPK3 | 7867 |
| 79 | Ataxin-7 | O15265 | 436 | ATXN7 | 6314 |
| 80 | Vitamin K-dependent protein S | P07225 | 437 | PROS1 | 5627 |
| 81 | ADP-ribosylation factor-like protein 6 | Q9HDF7 | 438 | ARL6 | 84100 |
| 82 | Interphotoreceptor matrix proteoglycan 2 | Q9BZV3 | 439 | IMPG2 | 50939 |
| 83 | IQ calmodulin-binding motif-containing protein 1 | Q15051 | 440 | IQCB1 | 9657 |
| 84 | Rhodopsin | P08100 | 441 | RHO | 6010 |
| 85 | Nephrocystin-3 | Q7Z494 | 442 | NPHP3 | 27031 |
| 86 | Clarin-1 | P58418 | 443 | CLRN1 | 7401 |
| 87 | Probable cationic amino acid transporter/Solute | Q8TBB6 | 444 | SLC7A14 | 57709 |
| | carrier family 7 member 14 | | | | |
| 88 | Choline-phosphate cytidylyltransferase A | P49585 | 445 | PCYT1A | 5130 |
| 89 | Centrosomal protein of 19 kDa | Q96LK0 | 446 | CEP19 | 84984 |
| 90 | Rod cGMP-specific 3′,5′-cyclic phosphodiesterase | P35913 | 447 | PDE6B | 5158 |
| | subunit beta | | | | |
| 91 | Wolframin | O76024 | 448 | WFS1 | 7466 |
| 92 | Homeobox protein HMX1 | Q9NP08 | 449 | HMX1 | 3166 |
| 93 | Ras-related protein Rab-28 | P51157 | 450 | RAB28 | 9364 |
| 94 | Coiled-coil and C2 domain-containing protein 2A | Q9P2K1 | 451 | CC2D2A | 57545 |
| 95 | Prominin-1 | O43490 | 452 | PROM1 | 8842 |
| 96 | Adhesion G protein-coupled receptor A3 | Q8IWK6 | 453 | ADGRA3 | 166647 |
| 97 | Death domain-containing protein 1 | Q6ZMT9 | 454 | DTHD1 | 401124 |
| 98 | WD repeat-containing protein 19 | Q8NEZ3 | 455 | WDR19 | 57728 |
| 99 | CGMP-gated cation channel alpha-1 | P29973 | 456 | CNGA1 | 1259 |
| 100 | CDGSH iron-sulfur domain-containing protein 2 | Q8NSK1 | 457 | CISD2 | 493856 |
| 101 | Microsomal triglyceride transfer protein large | P55157 | 458 | MTTP | 4547 |
| | subunit | | | | |
| 102 | Leucine-rich repeat, immunoglobulin-like domain | Q3SXY7 | 459 | LRIT3 | 345193 |
| | and transmembrane domain-containing protein 3 | | | | |
| 103 | Bardet-Biedl syndrome 7 protein | Q8IWZ6 | 460 | BBS7 | 55212 |
| 104 | Bardet-Biedl syndrome 12 protein | Q6ZW61 | 461 | BBS12 | 166379 |
| 105 | Major facilitator superfamily domain-containing | Q8NHS3 | 462 | MFSD8 | 256471 |
| | protein 8 | | | | |
| 106 | Serine/threonine-protein kinase PLK4 | O00444 | 463 | PLK4 | 10733 |
| 107 | Lecithin retinol acyltransferase | O95237 | 464 | LRAT | 9227 |
| 108 | Toll-like receptor 3 | O15455 | 465 | TLR3 | 7098 |
| 109 | Cytochrome P450 4V2 | Q6ZWL3 | 466 | CYP4V2 | 285440 |
| 110 | Spliceosome-associated protein CWC27 homolog | Q6UX04 | 467 | CWC27 | 10283 |
| 111 | Centrosomal protein POC5 | Q8NA72 | 468 | POC5 | 134359 |
| 112 | Versican core protein | P13611 | 469 | VCAN | 1462 |
| 113 | Adhesion G-protein coupled receptor V1 | Q8WXG9 | 470 | ADGRV1 | 84059 |
| 114 | COUP transcription factor 1 | P10589 | 471 | NR2F1 | 7025 |
| 115 | Mitochondrial outer membrane protein SLC25A46 | Q96AG3 | 472 | SLC25A46 | 91137 |
| 116 | Catenin alpha-1 | P35221 | 473 | CTNNA1 | 1495 |
| 117 | Histidine--tRNA ligase, cytoplasmic | P12081 | 474 | HARS1 | 3035 |
| 118 | Rod cGMP-specific 3′,5′-cyclic phosphodiesterase | P16499 | 475 | PDE6A | 5145 |
| | subunit alpha | | | | |
| 119 | Metabotropic glutamate receptor 6 | O15303 | 476 | GRM6 | 2916 |
| 120 | Serine/threonine-protein kinase MAK | P20794 | 477 | MAK | 4117 |
| 121 | Complement C2 | P06681 | 478 | C2 | 717 |
| 122 | Complement factor B | P00751 | 479 | CFB | 629 |
| 123 | Tubby-related protein 1 | O00294 | 480 | TULP1 | 7287 |
| 124 | Guanylyl cyclase-activating protein 1 | P43080 | 481 | GUCA1A | 2978 |
| 125 | Guanylyl cyclase-activating protein 2 | Q9UMX6 | 482 | GUCA1B | 2979 |
| 126 | Peripherin-2 | P23942 | 483 | PRPH2 | 5961 |
| 127 | Interphotoreceptor matrix proteoglycan 1 | Q17R60 | 484 | IMPG1 | 3617 |
| 128 | Protein eyes shut homolog | QST1H1 | 485 | EYS | 346007 |
| 129 | Collagen alpha-1(IX) chain | P20849 | 486 | COL9A1 | 1297 |
| 130 | Regulating synaptic membrane exocytosis protein | Q86UR5 | 487 | RIMS1 | 22999 |
| | 1 | | | | |
| 131 | Lebercilin | Q86VQ0 | 488 | LCA5 | 167691 |
| 132 | Elongation of very long chain fatty acids protein 4 | Q9GZR5 | 489 | ELOVL4 | 6785 |
| 133 | PR domain zinc finger protein 13 | Q9H4Q3 | 490 | PRDM13 | 59336 |
| 134 | Reticulon-4-interacting protein 1, mitochondrial | Q8WWV3 | 491 | RTN4IP1 | 84816 |
| 135 | Jouberin | Q8N157 | 492 | AHI1 | 54806 |
| 136 | Peroxisomal targeting signal 2 receptor | O00628 | 493 | PEX7 | 5191 |
| 137 | CCR4-NOT transcription complex subunit 9 | Q92600 | 494 | CNOT9 | 9125 |
| 138 | Aryl hydrocarbon receptor | P35869 | 495 | AHR | 196 |
| 139 | Kelch-like protein 7 | Q8IXQ5 | 496 | KLHL7 | 55975 |
| 140 | Retinitis pigmentosa 9 protein | Q8TA86 | 497 | RP9 | 6100 |
| 141 | Protein PTHB1 | Q3SYG4 | 498 | BBS9 | 27241 |
| 142 | Peroxisomal ATPase PEX1 | O43933 | 499 | PEX1 | 5189 |
| 143 | Tetraspanin-12 | O95859 | 500 | TSPAN12 | 23554 |
| 144 | Inosine-5′-monophosphate dehydrogenase 1 | P20839 | 501 | IMPDH1 | 3614 |
| 145 | Short-wave-sensitive opsin 1 | P03999 | 502 | OPN1SW | 611 |
| 146 | UPF0606 protein KIAA1549 | Q9HCM3 | 503 | KIAA1549 | 57670 |
| 147 | Retinitis pigmentosa 1-like 1 protein | Q8IWN7 | 504 | RP1L1 | 94137 |
| 148 | Disintegrin and metalloproteinase domain- | Q13443 | 505 | ADAM9 | 8754 |
| | containing protein 9 | | | | |
| 149 | Heparan-alpha-glucosaminide N-acetyltransferase | Q68CP4 | 506 | HGSNAT | 138050 |
| 150 | Oxygen-regulated protein 1 | P56715 | 507 | RP1 | 6101 |
| 151 | Alpha-tocopherol transfer protein | P49638 | 508 | TTPA | 7274 |
| 152 | Centrosome and spindle pole-associated protein 1 | Q1MSJ5 | 509 | CSPP1 | 79848 |
| 153 | Dynamin-like 120 kDa protein, mitochondrial | O60313 | 510 | OPA1 | 4976 |
| 154 | Peroxisome biogenesis factor 2 | P28328 | 511 | PEX2 | 5828 |
| 155 | Cyclic nucleotide-gated cation channel beta-3 | Q9NQW8 | 512 | CNGB3 | 54714 |
| 156 | Cilia- and flagella-associated protein 418 | Q96NL8 | 513 | CFAP418 | 157657 |
| 157 | Growth/differentiation factor 6 | Q6KF10 | 514 | GDF6 | 392255 |
| 158 | Regulating synaptic membrane exocytosis protein | Q9UQ26 | 515 | RIMS2 | 9699 |
| | 2 | | | | |
| 159 | Potassium voltage-gated channel subfamily V | Q8TDN2 | 516 | KCNV2 | 169522 |
| | member 2 | | | | |
| 160 | E3 ubiquitin-protein ligase Topors | Q9NS56 | 517 | TOPORS | 10210 |
| 161 | Centrosomal protein of 78 kDa | Q5JTW2 | 518 | CEP78 | 84131 |
| 162 | Inversin | Q9Y283 | 519 | INVS | 27130 |
| 163 | U4/U6 small nuclear ribonucleoprotein Prp4 | O43172 | 520 | PRPF4 | 9128 |
| 164 | Whirlin | Q9P202 | 521 | WHRN | 25861 |
| 165 | E3 ubiquitin-protein ligase TRIM32 | Q13049 | 522 | TRIM32 | 22954 |
| 166 | Toll-like receptor 4 | O00206 | 523 | TLR4 | 7099 |
| 167 | Cytoplasmic dynein 2 intermediate chain 2 | Q96EX3 | 524 | DYNC2I2 | 89891 |
| 168 | Programmed cell death protein 2 | Q16342 | 525 | PDCD2 | 5134 |
| 169 | Exosome complex component RRP4 | Q13868 | 526 | EXOSC2 | 23404 |
| 170 | Phosphatidylinositol polyphosphate 5-phosphatase | Q9NRR6 | 527 | INPP5E | 56623 |
| | type IV | | | | |
| 171 | Phytanoyl-CoA dioxygenese, peroxisomal | O14832 | 528 | PHYH | 5264 |
| 172 | Acyl-CoA-binding domain-containing protein | Q5T8D3 | 529 | ACBD5 | 91452 |
| 173 | Protocadherin-15 | Q96QU1 | 530 | PCDH15 | 65217 |
| 174 | Retinol-binding protein 3 | P10745 | 531 | RBP3 | 5949 |
| 175 | DNA excision repair protein ERCC-6 | Q03468 | 532 | ERCC6 | 2074 |
| 176 | Hexokinase-1 | P19367 | 533 | HK1 | 3098 |
| 177 | Cadherin-23 | Q9H251 | 534 | CDH23 | 64072 |
| 178 | Cadherin-related family member 1 | Q96JP9 | 535 | CDHR1 | 92211 |
| 179 | RPE-retinal G protein-coupled receptor | P47804 | 536 | RGR | 5995 |
| 180 | Kinesin-like protein KIF11 | P52732 | 537 | KIF11 | 3832 |
| 181 | Retinol-binding protein 4 | P02753 | 538 | RBP4 | 5950 |
| 182 | Cone cGMP-specific 3′,5′-cyclic phosphodiesterase | P51160 | 539 | PDE6C | 5146 |
| | subunit alpha | | | | |
| 183 | Paired box protein Pax-2 | Q02962 | 540 | PAX2 | 5076 |
| 184 | PDZ domain-containing protein 7 | Q9H5P4 | 541 | PDZD7 | 79955 |
| 185 | ADP-ribosylation factor-like protein 3 | P36405 | 542 | ARL3 | 403 |
| 186 | BBSome-interacting protein 1 | A8MTZ0 | 543 | BBIP1 | 92482 |
| 187 | Age-related maculopathy susceptibility protein 2 | P0C7Q2 | 544 | ARMS2 | 387715 |
| 188 | Serine protease HTRA1 | Q92743 | 545 | HTRA1 | 5654 |
| 189 | Ornithine aminotransferase, mitochondrial | P04181 | 546 | OAT | 4942 |
| 190 | Zinc finger protein 408 | Q9H9D4 | 547 | ZNF408 | 79797 |
| 191 | Tubby protein homolog | P50607 | 548 | TUB | 7275 |
| 192 | Transcriptional enhancer factor TEF-1 | P28347 | 549 | TEAD1 | 7003 |
| 193 | Harmonin | Q9Y6N9 | 550 | USHIC | 10083 |
| 194 | Transmembrane protein 216 | Q9P0N5 | 551 | TMEM216 | 51259 |
| 195 | Bestrophin-1 | O76090 | 552 | BEST1 | 7439 |
| 196 | Isoaspartyl peptidase/L-asparaginase | Q71266 | 553 | ASRGL1 | 80150 |
| 197 | Rod outer segment membrane protein 1 | Q03395 | 554 | ROM1 | 6094 |
| 198 | Bardet-Biedl syndrome 1 protein | Q8NFJ9 | 555 | BBS1 | 582 |
| 199 | Calcium-binding protein 4 | P57796 | 556 | CABP4 | 57010 |
| 200 | Low-density lipoprotein receptor-related protein 5 | O75197 | 557 | LRPS | 4041 |
| 201 | Calpain-5 | O15484 | 558 | CAPN5 | 726 |
| 202 | Unconventional myosin-VIIa | Q13402 | 559 | MYO7A | 4647 |
| 203 | Transmembrane protein 126A | Q9H061 | 560 | TMEM126A | 84233 |
| 204 | Frizzled-4 | Q9ULV1 | 561 | FZD4 | 8322 |
| 205 | Cytoplasmic dynein 2 light intermediate chain 1 | Q8TCX1 | 562 | DYNC2LI1 | 51626 |
| 206 | Centrosomal protein of 164 kDa | Q9UPV0 | 563 | CEP164 | 22897 |
| 207 | Complement C1q tumor necrosis factor-related | Q9BXJ0 | 564 | C1QTNF5 | 114902 |
| | protein 5 | | | | |
| 208 | Membrane frizzled-related protein | Q9BY79 | 565 | MFRP | 83552 |
| 209 | Voltage-dependent calcium channel subunit alpha- | Q7Z357 | 566 | CACNA2D4 | 93589 |
| | 2/delta-4 | | | | |
| 210 | Guanine nucleotide-binding protein G(I)/G(S)/G(T) | P16520 | 567 | GNB3 | 2784 |
| | subunit beta-3 | | | | |
| 211 | Retinal cone rhodopsin-sensitive cGMP 3′,5′-cyclic | Q13956 | 568 | PDE6H | 5149 |
| | phosphodiesterase subunit gamma | | | | |
| 212 | Collagen alpha-1(II) chain | P02458 | 569 | COL2A1 | 1280 |
| 213 | Matrix metalloproteinase-19 | Q99542 | 570 | MMP19 | 4327 |
| 214 | Retinol dehydrogenase 5 | Q92781 | 571 | RDH5 | 5959 |
| 215 | T-complex protein 1 subunit beta | P78371 | 572 | CCT2 | 10576 |
| 216 | Bardet-Biedl syndrome 10 protein | Q8TAM1 | 573 | BBS10 | 79738 |
| 217 | Centrosomal protein of 290 kDa | O15078 | 574 | CEP290 | 80184 |
| 218 | POC1 centriolar protein homolog B | Q8TC44 | 575 | POC1B | 282809 |
| 219 | Mevalonate kinase | Q03426 | 576 | MVK | 4598 |
| 220 | Intraflagellar transport protein 81 homolog | Q8WYA0 | 577 | IFT81 | 28981 |
| 221 | Mitochondrial translation release factor in rescue | Q9H3J6 | 578 | MTRFR | 91574 |
| 222 | Integral membrane protein 2B | Q9Y287 | 579 | ITM2B | 9445 |
| 223 | Retinoblastoma-associated protein | P06400 | 580 | RB1 | 5925 |
| 224 | RCC1 and BTB domain-containing protein 1 | Q8NDN9 | 581 | RCBT81 | 55213 |
| 225 | Rhodopsin kinase GRK1 | Q15835 | 582 | GRK1 | 6011 |
| 226 | X-linked retinitis pigmentosa GTPase regulator- | Q96KN7 | 583 | RPGRIP1 | 57096 |
| | interacting protein 1 | | | | |
| 227 | Neural retina-specific leucine zipper protein | P54845 | 584 | NRL | 4901 |
| 228 | Homeobox protein OTX2 | P32243 | 585 | OTX2 | 5015 |
| 229 | Retinol dehydrogenase 11 | Q8TC12 | 586 | RDH11 | 51109 |
| 230 | Retinol dehydrogenase 12 | Q96NR8 | 587 | RDH12 | 145226 |
| 231 | Tubulin polyglutamylase TTLL5 | Q6EMB2 | 588 | TTLL5 | 23093 |
| 232 | Spermatogenesis-associated protein 7 | Q9P0W8 | 589 | SPATA7 | 55812 |
| 233 | Tetratricopeptide repeat protein 8 | Q8TAM2 | 590 | TTC8 | 123016 |
| 234 | Fibulin-5 | Q9UBXS | 591 | FBLNS | 10516 |
| 235 | Transient receptor potential cation channel | Q7Z4N2 | 592 | TRPM1 | 4308 |
| | subfamily M member 1 | | | | |
| 236 | Gamma-tubulin complex component 4 | Q9UGJ1 | 593 | TUBGCP4 | 27229 |
| 237 | Sodium/potassium/calcium exchanger 1 | O60721 | 594 | SLC24A1 | 9187 |
| 238 | Photoreceptor-specific nuclear receptor | Q9Y5X4 | 595 | NR2E3 | 10002 |
| 239 | Bardet-Biedl syndrome 4 protein | Q96RK4 | 596 | BBS4 | 585 |
| 240 | Calcium and integrin-binding family member 2 | O75838 | 597 | CIB2 | 10518 |
| 241 | Retinaldehyde-binding protein 1 | P12271 | 598 | RLBP1 | 6017 |
| 242 | N-acetylglucosamine-1-phosphotransferase | Q9UJJ9 | 599 | GNPTG | 84572 |
| | subunit gamma | | | | |
| 243 | Intraflagellar transport protein 140 homolog | Q96RY7 | 600 | IFT140 | 9742 |
| 244 | Clusterin-associated protein 1 | Q96AJ1 | 601 | CLUAP1 | 23059 |
| 245 | ATP-binding cassette sub-family C member 6 | O95255 | 602 | ABCC6 | 368 |
| 246 | Ketimine reductase mu-crystallin | Q14894 | 603 | CRYM | 1428 |
| 247 | Battenin | Q13286 | 604 | CLN3 | 1201 |
| 248 | Zinc finger protein 423 | Q2M1K9 | 605 | ZNF423 | 23090 |
| 249 | Protein fantom | Q68CZ1 | 606 | RPGRIP1L | 23322 |
| 250 | Bardet-Biedl syndrome 2 protein | Q9BXC9 | 607 | BBS2 | 583 |
| 251 | ADP-ribosylation factor-like protein 2-binding | Q9Y2Y0 | 608 | ARL2BP | 23568 |
| | protein | | | | |
| 252 | Cyclic nucleotide-gated cation channel beta-1 | Q14028 | 609 | CNGB1 | 1258 |
| 253 | Cadherin-3 | P22223 | 610 | CDH3 | 1001 |
| 254 | Pre-mRNA-splicing factor ATP-dependent RNA | Q92620 | 611 | DHX38 | 9785 |
| | helicase PRP16 | | | | |
| 255 | A disintegrin and metalloproteinase with | Q8TE60 | 612 | ADAMTS18 | 170692 |
| | thrombospondin motifs 18 | | | | |
| 256 | Solute carrier family 38 member 8 | A6NNN8 | 613 | SLC38A8 | 146167 |
| 257 | Retinal guanylyl cyclase 1 | Q02846 | 614 | GUCY2D | 3000 |
| 258 | Pre-mRNA-processing-splicing factor 8 | Q6P209 | 615 | PRPF8 | 10594 |
| 259 | Aryl-hydrocarbon-interacting protein-like 1 | Q9NZN9 | 616 | AIPL1 | 23746 |
| 260 | Membrane-associated phosphatidylinositol | Q9BZ71 | 617 | PITPNM3 | 83394 |
| | transfer protein 3 | | | | |
| 261 | Protein unc-119 homolog A | Q13432 | 618 | UNC119 | 9094 |
| 262 | Probable G-protein coupled receptor 179 | Q6PRD1 | 619 | GPR179 | 440435 |
| 263 | Tectonic-like complex member MKS1 | Q9NXB0 | 620 | MKS1 | 54903 |
| 264 | Carbonic anhydrase 4 | P22748 | 621 | CA4 | 762 |
| 265 | Regulator of G-protein signaling 9 | O75916 | 622 | RGS9 | 8787 |
| 266 | Arylsulfatase G | Q96EG1 | 623 | ARSG | 22901 |
| 267 | pre-mRNA splicing regulator USH1G | Q495M9 | 624 | USH1G | 124590 |
| 268 | Photoreceptor disk component PRCD | Q00LT1 | 625 | PRCD | 768206 |
| 269 | Fascin-2 | O14926 | 626 | FSCN2 | 25794 |
| 270 | Retinal rod rhodopsin-sensitive cGMP 3′,5′-cyclic | P18545 | 627 | PDE6G | 5148 |
| | phosphodiesterase subunit gamma | | | | |
| 271 | Laminin subunit alpha-1 | P25391 | 628 | LAMA1 | 284217 |
| 272 | AFG3-like protein 2 | Q9Y4W6 | 629 | AFG3L2 | 10939 |
| 273 | Cone-rod homeobox protein | O43186 | 630 | CRX | 1406 |
| 274 | Receptor expression-enhancing protein 6 | Q96HR9 | 631 | REEP6 | 92840 |
| 275 | Retina and anterior neural fold homeobox protein | Q96IS3 | 632 | RAX2 | 84839 |
| | 2 | | | | |
| 276 | Complement C3 | P01024 | 633 | C3 | 718 |
| 277 | Rho guanine nucleotide exchange factor 18 | Q6ZSZ5 | 634 | ARHGEF18 | 23370 |
| 278 | Patatin-like phospholipase domain-containing | Q8IY17 | 635 | PNPLA6 | 10908 |
| | protein 6 | | | | |
| 279 | Regulator of G-protein signaling 9-binding protein | Q6ZS82 | 636 | RGS9BP | 388531 |
| 280 | Optic atrophy 3 protein | Q9H6K4 | 637 | OPA3 | 80207 |
| 281 | U4/U6 small nuclear ribonucleoprotein Prp31 | Q8WWY3 | 638 | PRPF31 | 26121 |
| 282 | Isocitrate dehydrogenase [NAD] subunit beta, | O43837 | 639 | IDH3B | 3420 |
| | mitochondrial | | | | |
| 283 | Pantothenate kinase 2, mitochondrial | Q9BZ23 | 640 | PANK2 | 80025 |
| 284 | Protein jagged-1 | P78504 | 641 | JAG1 | 182 |
| 285 | Molecular chaperone MKKS | Q9NPJ1 | 642 | MKKS | 8195 |
| 286 | Centrosomal protein kizuna | Q2M2Z5 | 643 | KIZ | 55857 |
| 287 | Lysophosphatidylserine lipase ABHD12 | Q8N2K0 | 644 | ABHD12 | 26090 |
| 288 | Kinesin-like protein KIF3B | O15066 | 645 | KIF3B | 9371 |
| 289 | Centrosome-associated protein CEP250 | Q9BV73 | 646 | CEP250 | 11190 |
| 290 | Pre-mRNA-processing factor 6 | O94906 | 647 | PRPF6 | 24148 |
| 291 | Cilia- and flagella-associated protein 410 | O43822 | 648 | CFAP410 | 755 |
| 292 | Dynamin-1-like protein | O00429 | 649 | DNM1L | 10059 |
| 293 | Metalloproteinase inhibitor 3 | P35625 | 650 | TIMP3 | 7078 |
| 294 | Intraflagellar transport protein 27 homolog | Q9BW83 | 651 | IFT27 | 11020 |
| 295 | Fibulin-1 | P23142 | 652 | FBLN1 | 2192 |
| 296 | MIEF1 upstream open reading frame protein | L0R8F8 | 653 | MIEF1 | |
| 297 | Aconitate hydratase, mitochondrial | Q99798 | 654 | ACO2 | 50 |
| 298 | Gamma-tubulin complex component 6 | Q96RT7 | 655 | TUBGCP6 | 85378 |
| 299 | Centriole and centriolar satellite protein | O75665 | 656 | OFD1 | 8481 |
| 300 | Retinoschisin | O15537 | 657 | RS1 | 6247 |
| 301 | Protein XRP2 | O75695 | 658 | RP2 | 6102 |
| 302 | Dystrophin | P11532 | 659 | DMD | 1756 |
| 303 | X-linked retinitis pigmentosa GTPase regulator | Q92834 | 660 | RPGR | 6103 |
| 304 | Nyctalopin | Q9GZU5 | 661 | NYX | 60506 |
| 305 | Xaa-Pro dipeptidase | P12955 | 662 | PEPC | 5184 |
| 306 | Norrin | Q00604 | 663 | NDP | 4693 |
| 307 | Voltage-dependent L-type calcium channel subunit | O60840 | 664 | CACNA1F | 778 |
| | alpha-1F | | | | |
| 308 | Phosphoglycerate kinase 1 | P00558 | 665 | PGK1 | 5230 |
| 309 | Rab proteins geranylgeranyltransferase | P24386 | 666 | CHM | 1121 |
| | component A 1 | | | | |
| 310 | Mitochondrial import inner membrane translocase | O60220 | 667 | TIMM8A | 1678 |
| | subunit Tim8 A | | | | |
| 311 | Ribose-phosphate pyrophosphokinase 1 | P60891 | 668 | PRPS1 | 5631 |
| 312 | Long-wave-sensitive opsin 1 | P04000 | 669 | OPN1LW | 5956 |
| 313 | Medium-wave-sensitive opsin 1 | P04001 | 670 | OPN1MW | 2652 |
| 314 | Short-wave-sensitive opsin 1 | P03999 | 671 | OPN1SW | 611 |
| 315 | Transcription factor A, mitochondrial | Q00059 | 672 | TFAM | 7019 |
| 316 | NADH-ubiquinone oxidoreductase chain 1 | P03886 | 673 | MT-ND1 | 4535 |
| 317 | NADH-ubiquinone oxidoreductase chain 2 | P03891 | 674 | MT-ND2 | 4536 |
| 318 | NADH-ubiquinone oxidoreductase chain 3 | P03897 | 675 | MT-ND3 | 4537 |
| 319 | NADH-ubiquinone oxidoreductase chain 4L | P03901 | 676 | MT-ND4L | 4539 |
| 320 | NADH-ubiquinone oxidoreductase chain 4 | P03905 | 677 | MT-ND4 | 4538 |
| 321 | NADH-ubiquinone oxidoreductase chain 5 | P03915 | 678 | MT-ND5 | 4540 |
| 322 | NADH-ubiquinone oxidoreductase chain 6 | P03923 | 679 | MT-ND6 | 4541 |
| 323 | ATP synthase subunit a | P00846 | 680 | MT-ATP6 | 4508 |
| 324 | ATP synthase protein 8 | P03928 | 681 | MT-ATP8 | 4509 |
| 325 | Cytochrome c oxidase subunit 1 | P00395 | 682 | MT-CO1 | 4512 |
| 326 | Cytochrome c oxidase subunit 3 | P00414 | 683 | MT-CO3 | 4514 |
| 327 | Cytochrome b | P00156 | 684 | MT-CYB | 4519 |
| 328 | Leucine--tRNA ligase, mitochondrial | Q15031 | 685 | LARS2 | 23395 |
| 329 | Nondiscriminating glutamyl-tRNA synthetase | Q5JPH6 | 686 | EARS2 | 124454 |
| | EARS2, mitochondrial | | | | |
| 330 | Lysine--tRNA ligase | Q15046 | 687 | KARS1 | 3735 |
| 331 | Histidine--tRNA ligase, mitochondrial | P49590 | 688 | HARS2 | 23438 |
| 332 | Serine--tRNA ligase, mitochondrial | Q9NP81 | 689 | SARS2 | 54938 |
| 333 | Probable proline--tRNA ligase, mitochondrial | Q7L3T8 | 690 | PARS2 | 25973 |
| 334 | Cyanocobalamin reductase/alkylcobalamin | Q9Y4U1 | 691 | MMACHC | 25974 |
| | dealkylase | | | | |
| 335 | POU domain, class 3, transcription factor 4 | P49335 | 692 | POU3F4 | 5456 |
| 336 | Ribosomal protein S6 kinase alpha-6 | Q9UK32 | 693 | RPSGKA6 | 27330 |
| 337 | Ciliogenesis and planar polarity effector 1 | Q9H799 | 694 | CPLANE1 | 65250 |
| 338 | Meckelin | Q5HYA8 | 695 | TMEM67 | 91147 |
| 339 | TRAF3-interacting protein 1 | Q8TDR0 | 696 | TRAF3IP1 | 26146 |
| 340 | Intraflagellar transport protein 74 homolog | Q96LB3 | 697 | IFT74 | 80173 |
| 341 | S phase cyclin A-associated protein in the | Q9BY12 | 698 | SCAPER | 49855 |
| | endoplasmic reticulum | | | | |
| 342 | Sodium channel and clathrin linker 1 | Q96NL6 | 699 | SCLT1 | 132320 |
| 343 | Protein TALPID3 | Q98VV6 | 700 | KIAA0586 | 9786 |
| 344 | Tectonic-2 | Q96GX1 | 701 | TCTN2 | 79867 |
| 345 | ADP-ribosylation factor-like protein 13B | Q3SXY8 | 702 | ARL13B | 200894 |
| 346 | B9 domain-containing protein 1 | Q9UPM9 | 703 | B9D1 | 27077 |
| 347 | B9 domain-containing protein 2 | Q9BPU9 | 704 | B9D2 | 80776 |
| 348 | C2 domain-containing protein 3 | Q4AC94 | 705 | C2CD3 | 26005 |
| 349 | Centrosomal protein of 41 kDa | Q9BYV8 | 706 | CEP41 | 95681 |
| 350 | Centrosomal protein of 104 kDa | O60308 | 707 | CEP104 | 9731 |
| 351 | Centrosomal protein of 120 kDa | Q8N960 | 708 | CEP120 | 153241 |
| 352 | Intraflagellar transport protein 172 homolog | Q9UG01 | 709 | IFT172 | 26160 |
| 353 | Katanin-interacting protein | O60303 | 710 | KATNIP | 23247 |
| 354 | Kinesin-like protein KIF7 | Q2M1P5 | 711 | KIF7 | 374654 |
| 355 | Retinal rod rhodopsin-sensitive cGMP 3′,5′-cyclic | O43924 | 712 | PDE6D | 5147 |
| | phosphodiesterase subunit delta | | | | |
| 356 | Tectonic-1 | Q2MV58 | 713 | TCTN1 | 79600 |
| 357 | Tectonic-3 | Q6NUS6 | 714 | TCTN3 | 26123 |
| 358 | Transmembrane protein 107 | Q6UX40 | 715 | TMEM107 | 84314 |
| 359 | Transmembrane protein 138 | Q9NPI0 | 716 | TMEM138 | 51524 |
| 360 | Transmembrane protein 231 | Q9H6L2 | 717 | TMEM231 | 79583 |
| 361 | Tetratricopeptide repeat protein 21B | Q7Z4L5 | 718 | TTC21B | 79809 |
| 362 | Nuclear receptor ROR-alpha | P35398 | 719 | RORA | 6095 |
| 363 | Beta-nerve growth factor | P01138 | 720 | NGF | 4803 |
| 364 | Collagen alpha-2(VIII) chain | P25067 | 721 | COL8A2 | 1296 |
| 365 | Solute carrier family 4 member 11 | Q8NBS3 | 722 | SLC4A11 | 83959 |
| 366 | Zinc finger E-box-binding homeobox 1 | P37275 | 723 | ZEB1 | 6935 |
| 367 | Keratin, type II cuticular Hb3 | P78385 | 724 | KRT83 | 3889 |
| 368 | Keratin, type I cytoskeletal 12 | Q99456 | 725 | KRT12 | 3859 |
| 369 | Transforming growth factor-beta-induced protein | Q15582 | 726 | TGFBI | 7045 |
| | ig-h3 | | | | |
| 370 | Tumor-associated calcium signal transducer 2 | P09758 | 727 | TACSTD2 | 4070 |
| 371 | Carbohydrate sulfotransferase 6 | Q9GZX3 | 728 | CHST6 | 4166 |
| 372 | Gelsolin | P06396 | 729 | GSN | 2934 |
| 373 | UbiA prenyltransferase domain-containing protein | Q9Y5Z9 | 730 | UBIAD1 | 29914 |
| | 1 | | | | |
| 374 | Decorin | P07585 | 731 | DCN | 1634 |
| 375 | 1-phosphatidylinositol 3-phosphate 5-kinase | Q9Y2I7 | 732 | PIKFYVE | 200576 |
| 376 | Transcription factor Ovo-like 2 | Q9BRP0 | 733 | OVOL2 | 58495 |
| 377 | Grainyhead-like protein 2 homolog | Q6ISB3 | 734 | GRHL2 | 79977 |

### Example 2: Evaluation of Expression of an HSV Reporter by Various Routes of Administration In Vivo

The present study evaluated the expression of an HSV reporter construct (termed HSV-mCherry) in the eye following various routes of administration to the eye.

All procedures conducted were in compliance with applicable animal welfare acts and were approved by the local Institutional Animal Care and Use Committee (IACUC). 18 healthy, 8-week-old female C57BL/6 mice were used in this study.

Mice were anesthesized with Ketamine and Xylazine via intraperitoneal injection at 85 mg/kg and 14 mg/kg, respectively.

Table 2 provides a synopsis of the experimental design. At 24-hours after the indicated route of administration of test article, plasma samples were collected then animals were sacrificed. Eyeballs were embedded in paraffin for downstream analysis.

TABLE 2
Study Design and Test Article (TA) Administration

| | | | Treatment | |
|---|---|---|---|---|
| Arm | Test System | Treatment | Details | Assessment |
| 1 | C57BL/6 | Test Article | Intracameral | Terminal plasma collection at |
| | mice (n = 3) | 1 | (IC), bilateral | Day 2 (24 hours ± 30 min post- |
| | | | 1 μl/eye | administration); |
| 2 | C57BL/6 | Test Article | Suprachoroidal | Terminal enucleation at Day 2 |
| | mice (n = 3) | 1 | (SCS), bilateral | with enucleation and fixation |
| | | | 1 μl/eye | of whole eyes (n = 3 mice/arms |
| 3 | C57BL/6 | Test Article | Intravitreal | 1-4), |
| | mice (n = 3) | 1 | (IVT), bilateral | or enucleation of eyes and |
| | | | 1 μl/eye | eyelids, separately with |
| 4 | C57BL/6 | Test Article | Subretinal, | fixation (n = 3 right eye/Arm 5 |
| | mice (n = 3) | 1 | bilateral | only), |
| | | | 1 μl/eye | or enucleation of eyes and |
| 5 | C57BL/6 | None | None | eyelids together with fixation |
| | mice (n = 3) | | | |

For intracameral administration, animals were anesthetized and the eyes were dilated. Following sedation and dilation, a pilot hole was made about 1.5 mm anterior to the limbus using a 31-gauge needle by inserting the needle to bevel depth into the aqueous humor. A 1 μl air bubble was first injected into the anterior chamber through the pilot hole using a syringe and a 33-gauge beveled needle to prevent reflux. Next, a total volume of 1 μl of test article (1.16×10¹⁰ PFU/mL) per eye was injected over a period of 5-10 minutes. Care was taken to avoid damaging the iris and lens. Following injection, saline was used to rinse and hydrate the eye. Antibiotic ointment was then applied to the injection site and the animals were monitored until fully awake.

For suprachoroidal administration, eyes were dilated with 1% cyclopentolate hydrochloride ophthalmic solution and 10% phenylephrine prior to injection. Following administration of Ketamine and Xylazine, the animals were placed on a regulated heating pad, and the temporal region of sclera was visualized under magnification. An 8 mm 31-gauge insulin syringe was used to puncture the sclera about 1-1.5 mm below the corneal limbus, avoiding tears through the retinal pigment epithelial (RPE)/Choroid, retina, or vitreous body. The suprachoroidal injections were performed using a 10 μl syringe with a 33-gauge 45 degree beveled needle inserted through the scleral puncture, with the bevel aimed upward toward the cornea to complete the scleral tear. A total volume of 1 μL of test article (1.16×10¹⁰ PFU/mL) was delivered per eye. Successful injections were assessed through a dissection microscope and lacked any blood in the vitreous or light reflection from test agent flowing into the vitreous space or scratches of lens.

For intravitreal administration, animals were anesthetized and eyes were dilated. Following sedation and dilation, a total volume of 5 μL of test article (1.16×10¹⁰ PFU/mL) per eye was injected into the vitreous at the pars plana using a syringe and a 33-gauge needle.

For subretinal injection, mouse eyes were dilated and the animals were anesthetized. The mice were placed on a regulated heating pad, and the posterior pole was visualized under magnification. A 12.7 mm 30-gauge insulin syringe was used to puncture the cornea just above the corneal limbus, avoiding any contact with the sclera and lens. The transvitreal subretinal injections were performed using a 10 μL syringe with a 33-gauge blunt needle inserted through the corneal puncture across the vitreous, with the shaft aimed at the back of the eyecup, avoiding any trauma to the lens or iris. A total volume of 1 μL of test article (1.16×10¹⁰ PFU/mL) was delivered.

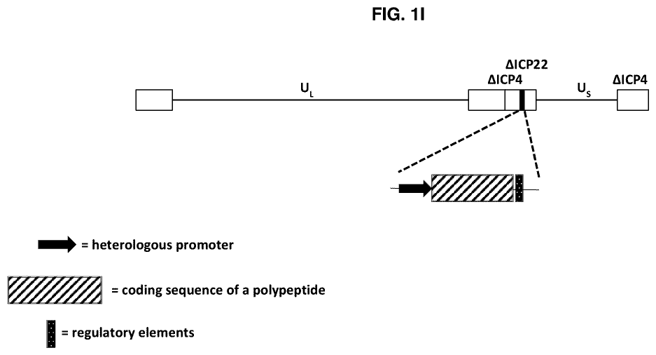

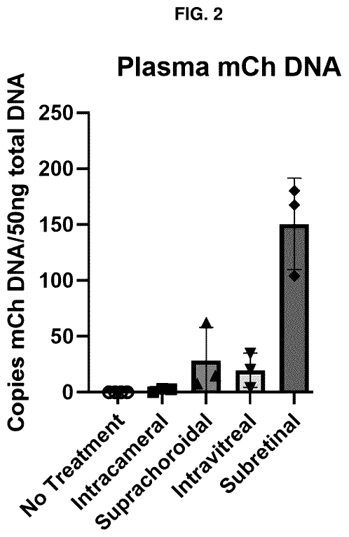

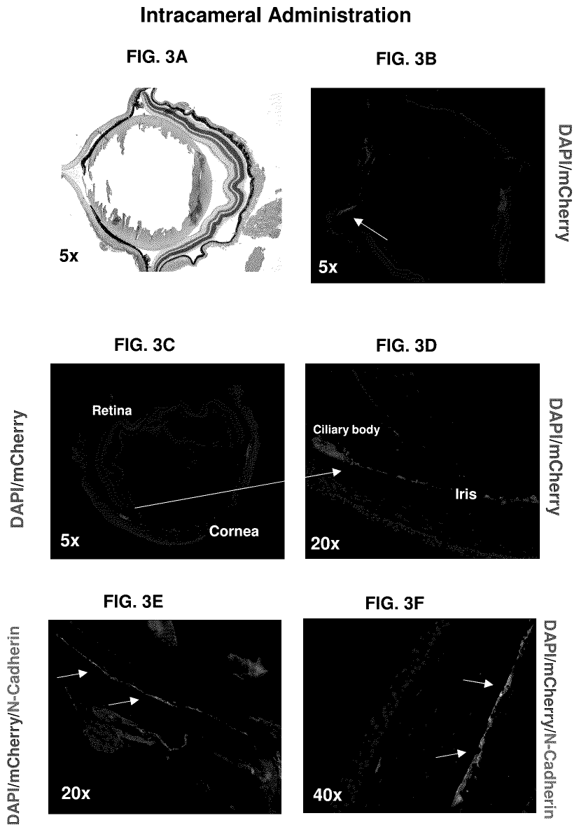

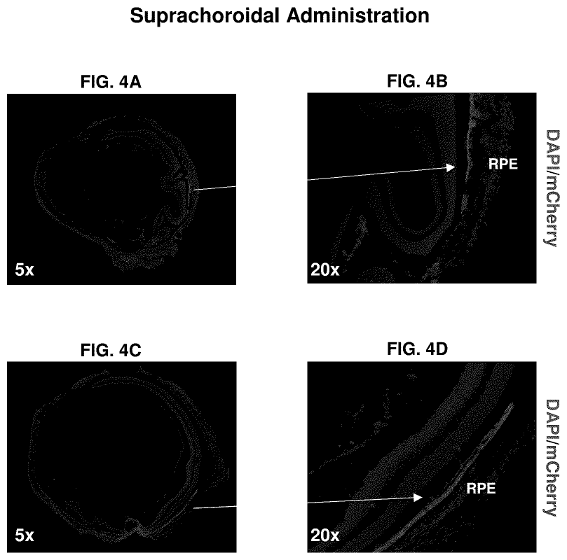

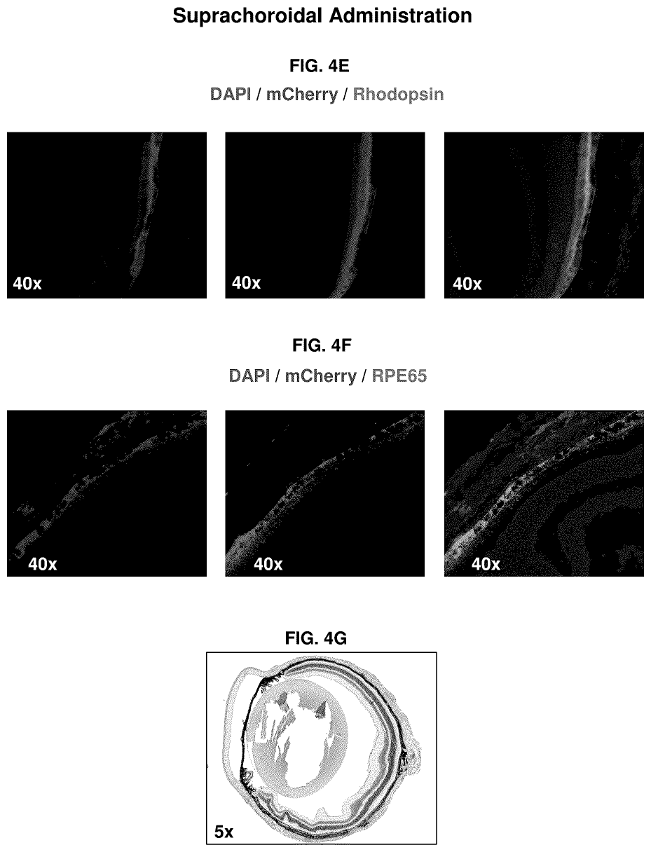

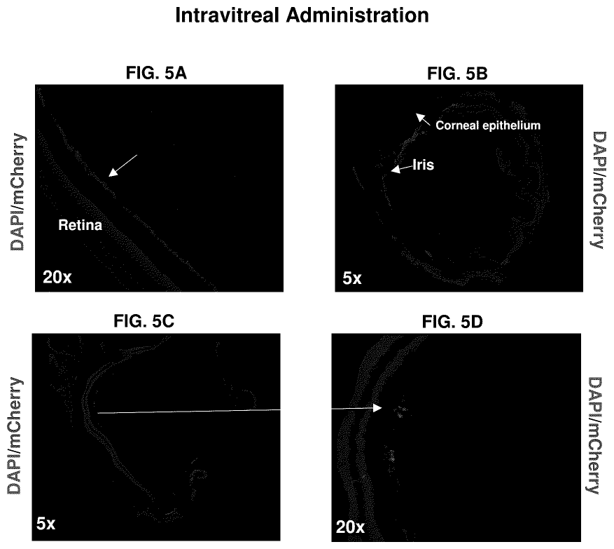

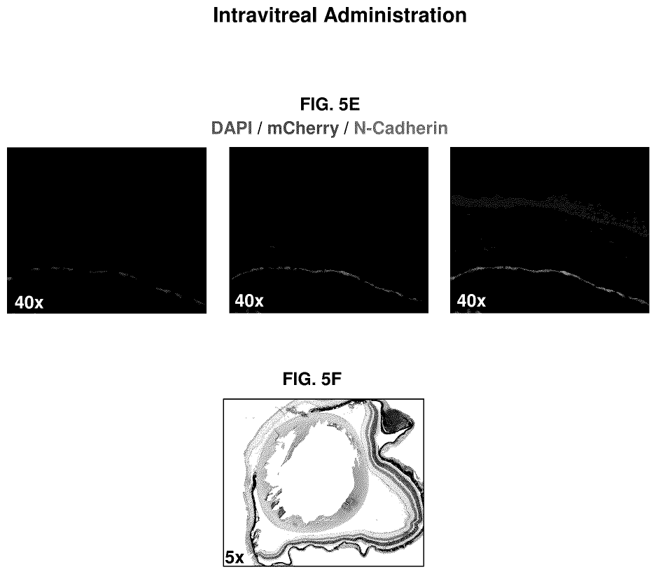

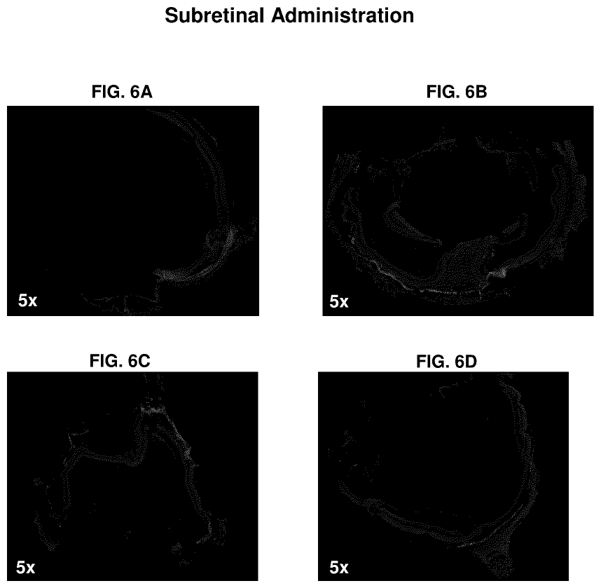

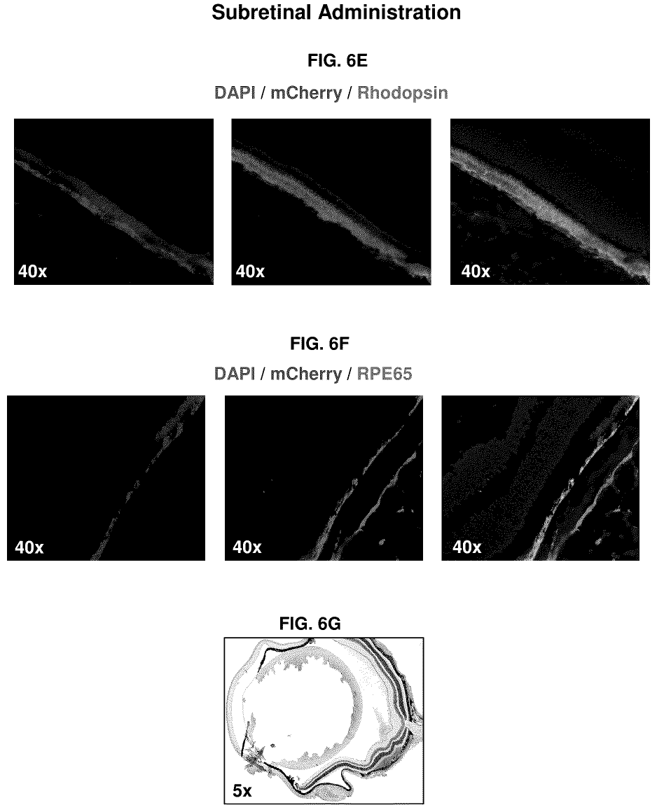

Systemic vector (HSV-mCherry) exposure was assessed in plasma samples collected 24-hours after test article administration (FIG. 2). As shown in FIG. 2, minimal systemic vector DNA was detected (e.g. at or below the limit of detection of about 100 copies of mCherry DNA/50 ng total DNA). Further, and as determined by hematoxylin and eosin (H&E) staining, very few inflammatory cells were observed in the suprachoroidal (FIG. 4G) and subretinal (FIG. 6G) groups, while the intracameral (FIG. 3A) and intravitreal (FIG. 5F) groups showed mild inflammatory cell infiltration.

Next, HSV-mCherry expression was assessed 24-hours post intracameral, suprachoroidal, intravitreal, or subretinal administration. As shown in FIGS. 3A-3F, intracameral administration resulted in reporter expression in at least the ciliary body, iris, and/or corneal endothelial cells. Reporter expression was observed across the retina including in retinal pigment epithelial (RPE) cells and/or the photoreceptor retinal area following suprachoroidal administration (FIGS. 4A-4F). FIGS. 5A-5E show reporter expression in the ciliary body, iris, corneal epithelium, corneal endothelium, and/or the outside layer of retina (potentially retinal ganglion cells) following intravitreal administration. Finally, subretinal administration resulted in reporter expression in the retinal area across the retina (not just localized to the injection site) including in at least the photoreceptor retinal area and RPE cells (FIGS. 6A-6F). Taken together, these data indicate reporter expression via various routes of administration to the eye. Importantly, no fibrosis, necrosis, or other gross physiologic defects (as indicated by histology) were noted in the eye 24 hours after any route of vector administration, indicating the vector is safe in the eye.

Taken together, these data suggest the HSV-based vector described herein was capable of transducing multiple clinically-relevant cell types in the eye, including both photoreceptors and retinal pigment epithelial cells in the retina, with little-to-no inflammation.

### Example 3: In Vitro Analysis of a Modified Herpes Simplex Virus Human Encoding Beta-Nerve Growth Factor (bNGF)

The following example describes the use of a recombinant herpes simplex virus type 1 (HSV-1) that successfully encoded human beta-nerve growth factor (bNGF) (see e.g. SEQ ID NOs: 720, 735, 736, 741, 742, and 743) and expressed full-length human bNGF (see SEQ ID NOs: 363, 739, and 740) protein.

A recombinant HSV-1 was engineered to incorporate a human pro-NGF (see SEQ ID NO: 735) expression cassette, and a recombinant HSV-1 was engineered to incorporate a human NGF (see SEQ ID NO: 736) expression cassette. Both recombinant HSV-1 vectors contained a heterologous promoter and polyA sequence (see Example 1). Viral plaques putatively containing the human NGF cassettes were picked and screened by infection in a complementing cell line. High expressing clones, termed HSV-proNGF and HSV-sNGF, were subsequently selected for additional in vitro analysis.

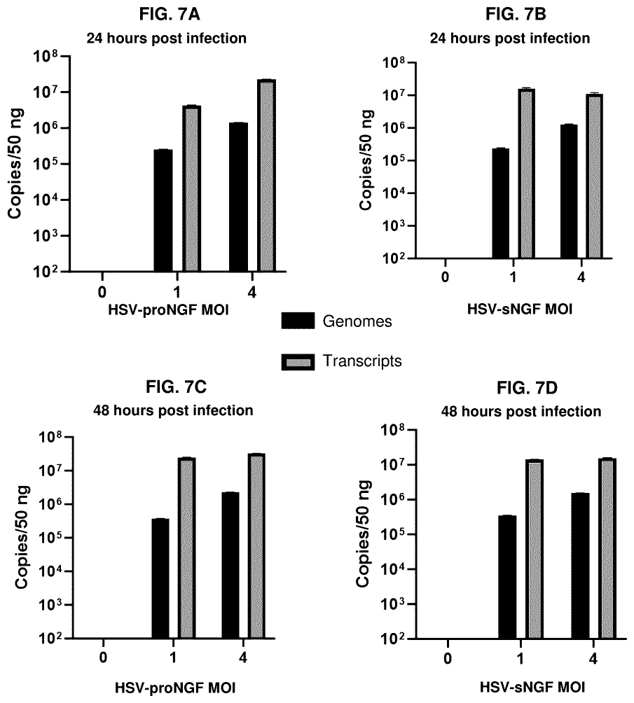

Initially, vector genomes and NGF transcripts derived from HSV-proNGF and HSV-sNGF were measured in primary corneal epithelial cells. Briefly, sequenced titered material was used to infect primary corneal epithelial cells in 6-well plates at a multiplicity of infection (MOI) of 0, 1, or 4. DNA and RNA were isolated 24 or 48 hours post infection (hpi) from cellular pellets and vector genomes (DNA) and NGF transcripts (RNA) was measured by qPCR and qRT-PCR, respectively. As shown in FIGS. 7A-7D, a direct relationship was observed in vector genome and NGF transcript quantification with both HSV-proNGF (FIGS. 7A & 7C) and HSV-sNGF (FIGS. 7B & 7D) infection of primary corneal epithelial cells dose at 24 hpi (FIGS. 7A-7B) and 48 hpi (FIGS. 7C-7D).

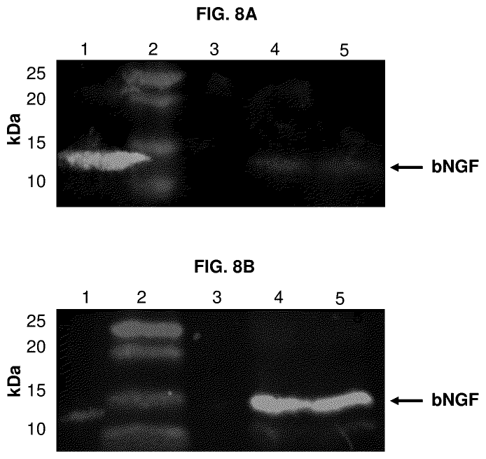

Next, western blot analysis was conducted to measure expression of correctly processed bNGF protein from the engineered HSV-proNGF vector. Primary corneal epithelial cells were mock infected with vehicle control or were infected with HSV-proNGF at an MOI of 1 or 4. 48 hours post-infection, media was collected and normalized for protein content by ELISA (FIG. 8A: 8-10 ng of total protein was loaded; FIG. 8B: 115-140 ng of total protein was loaded). Samples were run on a 4-20% acrylamide gel. Recombinant human bNGF was loaded on the gel as a positive control. As shown in FIGS. 8A-8B, while no human bNGF was detected in the uninfected control cells, robust expression of human bNGF was observed after infection with HSV-proNGF.

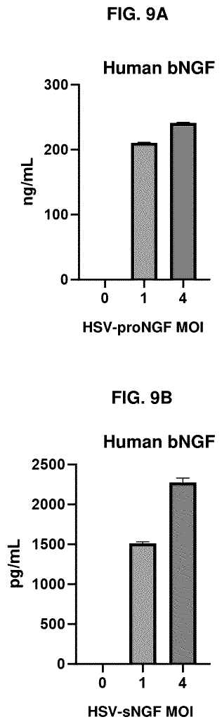

Because NGF is a naturally secreted protein, primary corneal epithelial cell culture supernatants were also harvested and tested for the presence of the HSV derived human bNGF by ELISA. In line with the western blot data, HSV derived human bNGF protein was detected in the supernatants of primary corneal epithelial cells infected with HSV-proNGF and HSV-sNGF at the MOIs tested (FIGS. 9A-9B), suggesting that the full length human protein was being properly processed/secreted after expression from the recombinant vector.

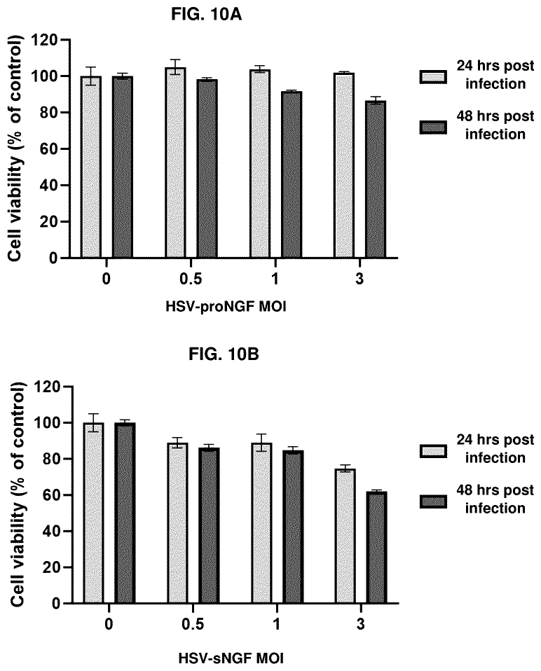

Next, an MTT assay was performed to measure cell toxicity following infection of primary corneal epithelial cells with HSV-proNGF or HSV-sNGF. Briefly, primary corneal epithelial cells were plated at 2×10⁴ cells/well in a 96-well plate. 24-hours after plating, cells were infected with a control virus, HSV-proNGF, or HSV-sNGF at an MOI of 0, 0.5, 1, or 3. An MTT assay was performed 24- and 48-hours post-infection. As shown in FIGS. 10A-10B, cell viability remained intact at the time points and MOIs tested, suggesting minimal cellular toxicity after infection with HSV-proNGF or HSV-sNGF.

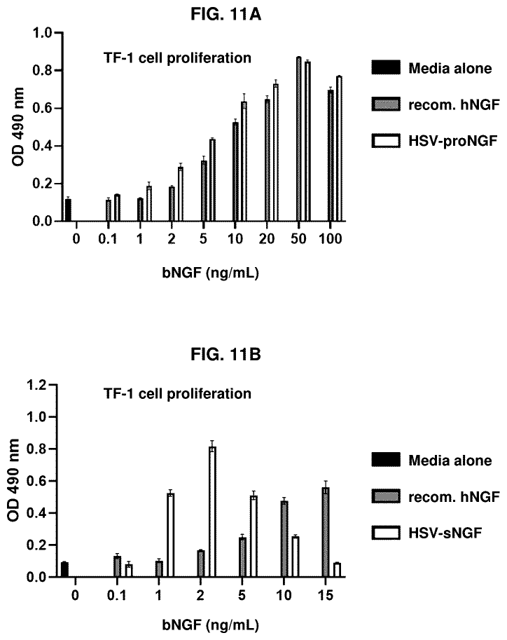

Finally, vector derived bNGF function was determined based on a TF-1 cell proliferation activity assay. Briefly, TF-1 cells were plated at 1.4×10⁴ cells/well in a 96-well plate. The TF-1 cells were then placed in varying concentrations of recombinant human bNGF protein, or in media from HEK-293T cells which had been infected with HSV-proNGF or HSV-sNGF (NGF levels in media from infected HEK-293T cells was determined by ELISA). TF-1 cells were grown for 72 hours following bNGF addition, and a Cell Titer Assay was performed to measure TF-1 cell proliferation as a function of bNGF activity. As shown in FIGS. 11A-11B, bNGF derived from HSV-proNGF (FIG. 11A) and HSV-sNGF (FIG. 11B) promoted the proliferation of TF-1 cells in a comparable or greater fashion to that of recombinant human bNGF.

Take together, the data presented in this example indicated that recombinant HSV-1 vectors HSV-proNGF and HSV-sNGF efficiently infected primary cells of the eye and were capable of expressing the human NGF transgenes encoded therein. Further, the data indicated that the exogenous human protein was subsequently (properly) secreted from infected cells. Further, the recombinant human bNGF protein made from the recombinant herpes simplex virus was at least as, if not more, bioactive as commercially available recombinant bNGF protein without inducing cellular toxicity.

Without wishing to be bound by theory, it is believed that these data further support the use of engineered herpes simplex viruses as novel, targeted, broadly applicable gene therapy vectors for the treatment of conditions involving the eye.

### Example 4: Evaluation of Expression of an HSV Reporter Topically Applied In Vivo Following Corneal Injury

The present study evaluated the expression of a topically applied HSV reporter construct (termed HSV-mCHerry) in corneal wounded eyes.

All procedures conducted were in compliance with applicable animal welfare acts and were approved by the local Institutional Animal Care and Use Committee (IACUC). 15 healthy, 8-week-old female C57BL/6 mice were used in this study.

Mice were anesthetized with Ketamine and Xylazine via intraperitoneal injection at 85 mg/kg and 14 mg/kg, respectively.

Table 3 provides a synopsis of the experimental design. At 24-hours after topical administration of test article, the animals were sacrificed. Test article administration occurred immediately following the indicated corneal wounding procedure and while the animals were still anesthetized. Eyeballs were either embedded in paraffin or snap frozen for downstream analysis.

TABLE 3
Study Design and Test Article (TA) Administration

| | Test | | | Treatment | |
|---|---|---|---|---|---|
| Arm | System | Induction | Test Article | Details | Assessment |
| 1 | C57BL/6 | Bilateral 1 | HSV-mCherry | Topical, | Terminal blood collection at |
| | mice (n = 4; | mm | | bilateral | Day 2 (24 hours ± 30 min |
| | n = 8 eyes) | mechanical | | 3 μl/eye | post-administration); |
| | | wounding | | | Terminal enucleation of |
| 2 | C57BL/6 | Bilateral 1 | HSV-mCherry | Topical, | whole eyes with fixation and |
| | mice (n = 4; | mm | | bilateral | paraffin embedding (n = 5 |
| | n = 8 eyes) | (deeper) | | 3 μl/eye | eyes/arms 1-3), |
| | | mechanical | | | Terminal collection of |
| | | wounding | | | corneal epithelium and eyes |
| 3 | C57BL/6 | Bilateral | HSV-mCherry | Topical, | without epithelium collected |
| | mice (n = 4; | crosshatch | | bilateral | in RNAlater (n = 3 eye/arms 1- |
| | n = 8 eyes) | wound to | | 3 μl/eye | 3; n = 6 eyes, Arm 4) |
| | | both eyes | | | |
| 4 | C57BL/6 | None | None | None | |
| | mice (n = 3) | | | | |

Baseline corneal fluorescein staining was conducted in order to exclude animals with preexisting corneal injury. On day 1, bilateral 1 mm mechanical debridement wounds were created by an Alger Brush II, or a 5×5 cornea-wide crosshatch wound was created. Also at day 1, and immediately after corneal wounding and prior to test article administration, fluorescein staining of one eye in each arm was conducted to confirm corneal wounding. Topical administration of test article (3 μl of HSV-mCherry at a concentration of 1.16×10¹⁰ PFU/mL) was applied to the wounded eyes, and 24-hours post test article administration, terminal collection of whole eyes with fixation and paraffin embedding occurred. Alternatively, terminal enucleation of whole eyes with dissection of corneal epithelium and processing of whole eyes and epithelium, separately, in RNAlater was conducted.

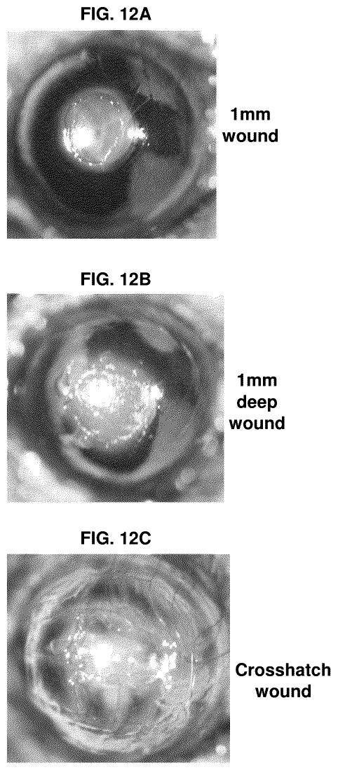

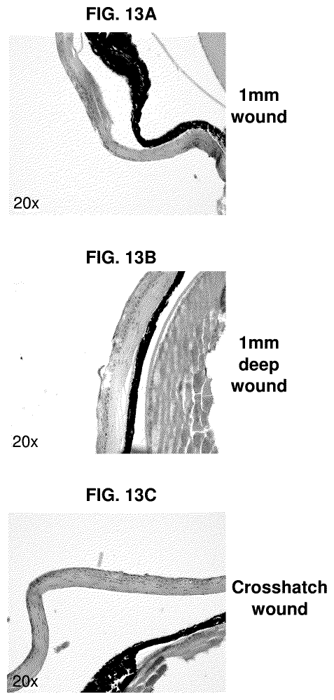

As shown in FIGS. 12A-12C, fluorescein staining showed robust corneal wounding in all three models tested (FIG. 12A, 1 mm wound; FIG. 12B, 1 mm deep wound; and FIG. 12C, crosshatch wound). Corneal wounding was further confirmed by H&E staining (FIGS. 13A-13C) which showed disruption of the outer corneal epithelial layer in all wound models tested. In addition, the 1 mm deep wound group also showed disruption of the lower stromal layer.

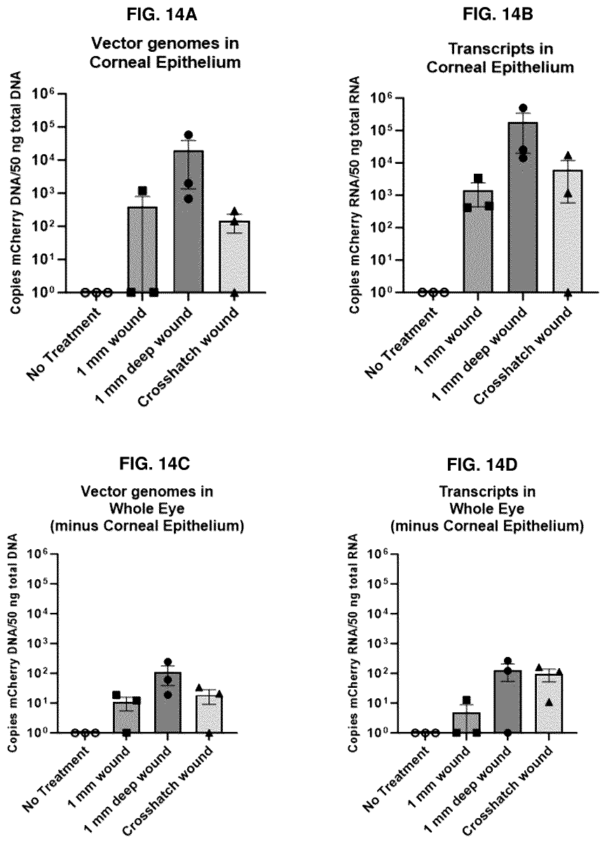

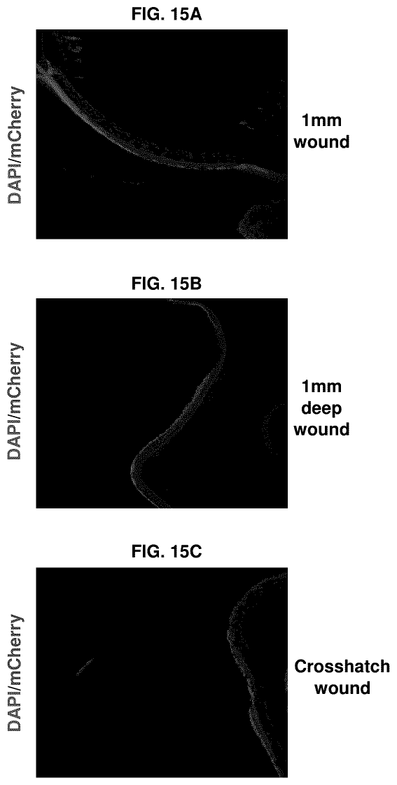

Next, vector genomes (FIGS. 14A and 14C) and mCherry transcripts (FIGS. 14B and 14D) were measured by qPCR and qRT-PCR, respectively, 24-hours post topical administration to corneal wounded eyes. As shown in FIGS. 14A-14B, topical application to corneal wounded eyes resulted in reporter expression in the corneal epithelium in all wound types tested. Further, vector genomes (FIG. 14C) and mCherry transcripts (FIG. 14D) were detected in the whole eye (minus the corneal epithelium), albeit at lower levels compared to the corneal epithelium. Importantly, mCherry transcripts were not detected in whole blood, suggesting no vector escape into the blood at 24 hours post topical application of test article to wounded eyes (data not shown). In addition, and as shown in FIGS. 15A-15C, topical administration to corneal wounded eyes resulted in reporter expression spanning the outer corneal epithelial layer with some dissemination of reporter signal deeper into the epithelial cell layers.

Taken together, these data suggest the HSV-based vector described herein was capable of transducing corneal wounded eyes following topical application. Without wishing to be bound by theory, it is believed that these data further support the use of engineered herpes simplex viruses as novel, targeted, broadly applicable gene therapy vectors for the treatment of conditions involving corneal disease (e.g. neurotrophic keratitis).

### Example 5: In Vitro and In Vivo Analysis of a Modified Herpes Simplex Virus Encoding Human Retinal-Specific Phospholipid-Transporting ATPase (ABCA4)

ABCA4 is a membrane-bound translocase located in photoreceptor cells on the edge of rod and cone outer segment discs and to a lesser extent in retinal pigment epithelial (RPE) cells. The major substrate for ABCA4 translocase activity is N-retinylidene-phosphatidylethanolamine (N-ret-PE), a byproduct that forms in photoreceptors during light perception and whose conversion to 11-cis-retinal is essential for continuation of the visual cycle and maintenance of visual acuity. ABCA4 is therefore critical for preventing accumulation of retinal and toxic bisretinoid compounds that may form through reactions with other substrates in photoreceptors. This also avoids buildup of these toxic compounds in the retinal pigment epithelium (RPE) during the natural process of continuous phagocytosis of outer segment discs by RPE cells, where additional reactions with N-ret-PE can occur to form bisretinoids. One toxic biproduct is the metabolite pyridium bisretinoid (A2E), a precursor of lipofuscin in RPE cells that can accumulate and lead to RPE dysfunction and loss with subsequent photoreceptor death. A2E can damage cells through photooxidative processes and disruption of membranes, and thus its accumulation through loss or dysfunction of ABCA4 can have detrimental effects on retinal structure and function.

Genetic variants in the ABCA4 gene cause Stargardt disease, an autosomal recessive retinal disease with an estimated incidence of between 1 in 8,000-10,000. Stargardt is the most prevalent inherited macular dystrophy in both children and adults; onset is most common during childhood, but it can also occur into adulthood with variable prognosis. Some key diagnostic features include macular atrophy, the presence of yellow-white lipofuscin flecks at the level of the RPE, and bilateral central vision loss, which may worsen over time with disease progression as retinal function and structural integrity are lost. The age at clinical presentation, rate of progression, and prognosis are all highly variable for Stargardt disease, making it challenging to treat once diagnosed. Patients may be advised to avoid exposure to ultraviolet light and vitamin A supplementation based on studies in Abca4−/− mice suggesting that these factors accelerate retinal degeneration. Currently, there are no FDA-approved therapeutic options for Stargardt disease. Several different pharmacological interventions, including visual cycle modulators (VCMs) that prevent accumulation of toxic vitamin A dimers, are currently undergoing Phase 2 and 3 clinical trials. Because Stargardt disease is inherited in an autosomal recessive manner, clinical phenotypes are caused by ABCA4 protein insufficiencies linked to one of up to 900 genetic variants identified in this patient population; introduction of functional gene copies could therefore overcome these deficiencies and restore vision for affected individuals. Gene therapy is a promising option because retinal degeneration and subsequent vision loss is gradual in Stargardt patients, allowing time for therapeutic action. Viral vectors, such as adeno-associated viruses (AAV) and lentiviruses (LV), are limited by the genome size they can carry or the risk of carcinogenicity associated with insertional mutagenesis, respectively. Although both AAV and LV vectors have been attempted in the clinic, they have seen little success. The use of an HSV-1-based vector overcomes the limitations of genome size and presents no risk of insertional mutagenesis, making it a novel approach compared to current viral gene therapies. The use of an HSV-1-based vector platform technology capable of packaging full-length human ABCA4 and subsequently stimulating its robust expression in both photoreceptors and RPE cells upon administration to the eye would therefore represent an innovative molecular correction strategy for the treatment of Stargardt disease.

This study coupled the beneficial properties inherent to HSV-1, including its episomal lifecycle, high payload capacity, transduction efficiency, tropism for myriad human cell types, and natural immune evasiveness, with a genetic modification to render a vector platform that is both replication-defective and less cytotoxic. Accordingly, described herein is the use of a replication-defective, non-integrating HSV-1-based vector that has been engineered to deliver functional, full-length human ABCA4 (termed HSV-ABCA4) directly to relevant cell types of the patient's retina via suprachoroidal injection.

### In Vitro

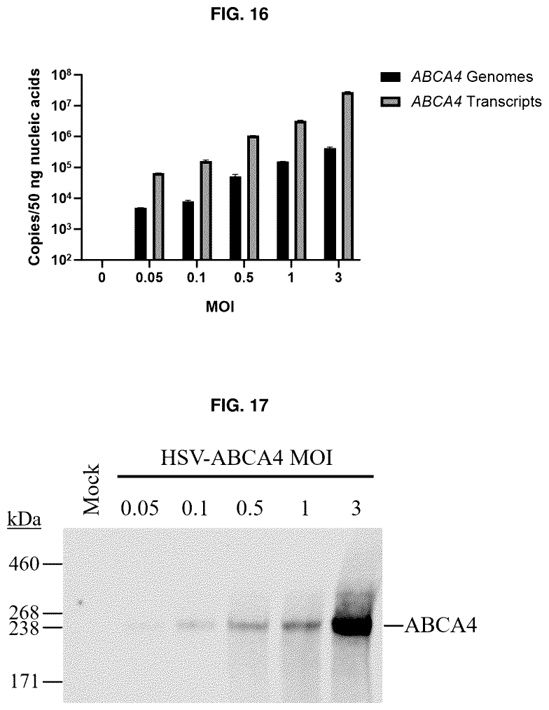

The HSV-ABCA4 vector was first tested for its ability to transduce and deliver its transgene to clinically relevant primary human RPE cells (ARPE-19). ARPE-19 cells are a spontaneously arising RPE cell line derived from the normal eyes of a 19-year-old male donor (.ARPE-19 cells were transduced with HSV-ABCA4 at multiplicity of infection (MOI) of 0.05, 0.1, 0.5, 1, and 3. Cell pellets were collected 24 hours post-transduction for nucleic acid isolation. Vector genome copy number and ABCA4 transcript expression were quantified by qPCR and qRT-PCR analyses, respectively. As shown in FIG. 16, ARPE-19 cells were found to be efficiently transduced by the vector, and robust expression of the human transgene was observed.

Next, HSV-ABCA4 was tested to confirm its ability to express full-length human ABCA4 protein in ARPE-19 cells. Again, cells were transduced at an MOI of 0.05, 0.1, 0.5, 1, and 3, and cell lysates were assessed for human ABCA4 protein expression via western blot 48 hours post-transduction. Mock transduced ARPE-19 cells, used as a negative control for basal ABCA4 expression in this cell line, were below the limit of detection. Transduction of HSV-ABCA4 in ARPE-19 cells resulted in dose-dependent expression of full-length ABCA4 protein (FIG. 17).

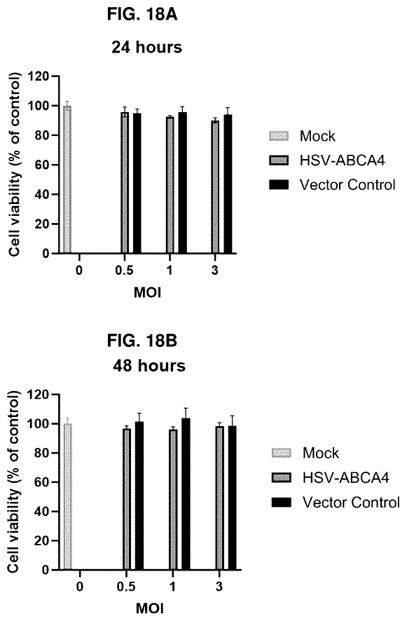

To investigate the potential cytotoxic effects of the recombinant virus, dose-dependent toxicity of HSV-ABCA4 was evaluated by Mosmann's Tetrazolium Toxicity (MTT) assay on cultured human ARPE-19 cells 24 hours (FIG. 18A) and 48 hours (FIG. 18B) post-transduction. No significant dose-dependent effects on cell metabolism, physiology, or toxicity were observed in these clinically relevant cells, as compared to both vector control and mock treated (MOI 0) cells (set to 100% viability within each assay).

### In Vivo

Mice lacking ABCA4 (Abca4^{-/-} mice) have significantly increased levels of A2E and other lipofuscin fluorophores in the retina and RPE. Although ABCA4 is largely localized to photoreceptors, it is also present in RPE cells; mice that have been modified to only express ABCA4 in RPE cells but not in photoreceptors showed a partial rescue in photoreceptor degeneration and decreased lipofuscin buildup compared to mice with complete ABCA4 knockout. This suggests that both RPE and photoreceptor cells should be targets of molecular correction in Abca4-mediated retinal dystrophies.

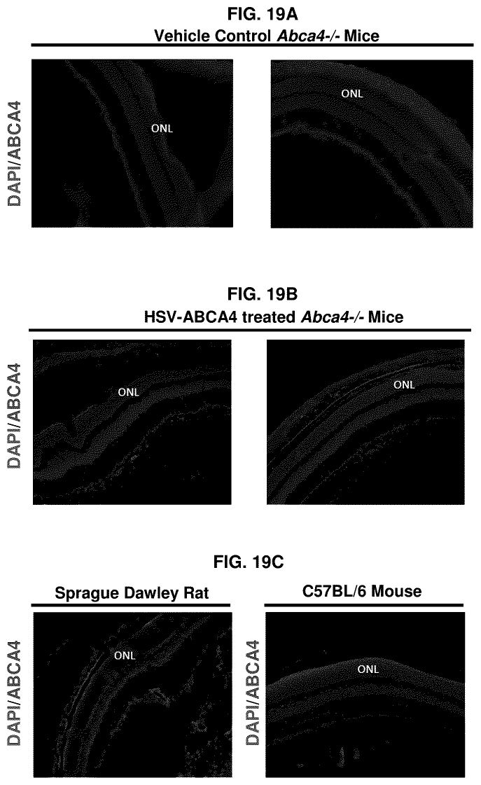

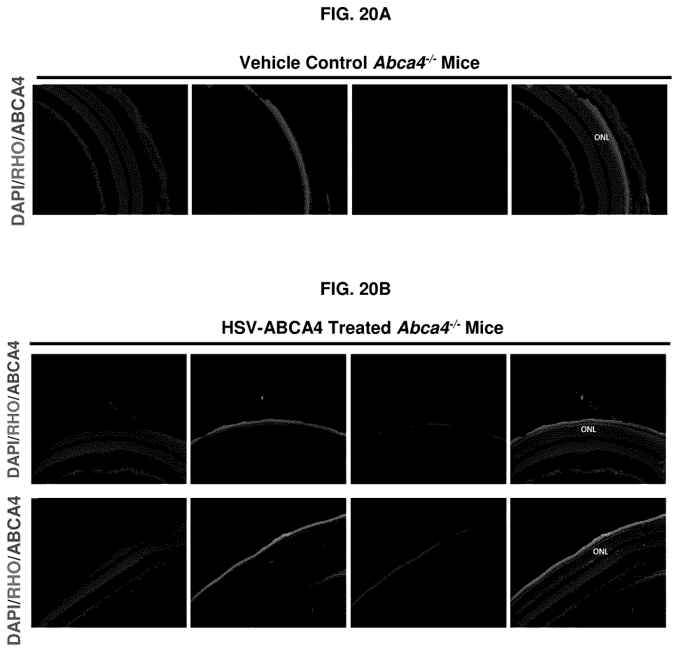

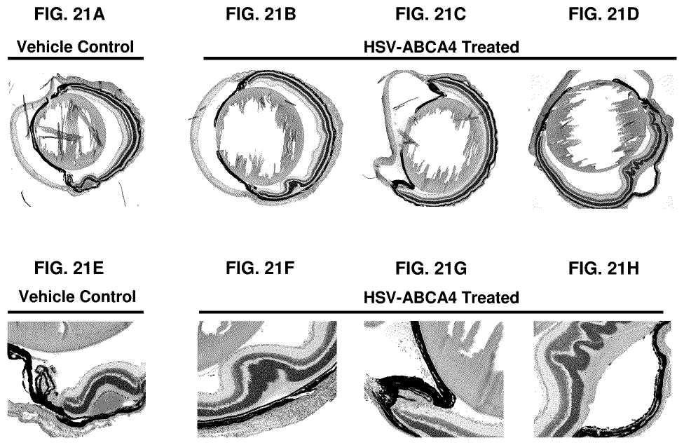

The ability of HSV-ABCA4 to deliver ABCA4 protein to relevant cells in the retina was explored through vector administration to Abca4^{-/-} mice. Four-week-old Abca4−/− mice were treated via a single suprachoroidal injection to each eye with either vehicle control or HSV-ABCA4. All eyes were harvested at 24 hours post-injection to investigate ABCA4 protein levels by immunofluorescence (IF) staining. As expected, all vehicle control treated eyes were free of ABCA4 IF staining (FIG. 19A). Molecular replacement of ABCA4 was seen in the retinas of Abc4^{-/-} mice treated with HSV-ABCA4 (FIG. 19B). The ABCA4 staining pattern in HSV-ABCA4 treated Abca4^{-/-} mouse eyes mirrored that seen in wild-type rodent eyes (FIG. 19C). Colocalization of human ABCA4 and murine rhodopsin confirmed transgene delivery to clinically relevant photoreceptor cells in the retina upon suprachoroidal injection of HSV-ABCA4 (FIGS. 20A-20B).

To discern the level of vector escape from the eye after suprachoroidal administration of HSV-ABCA4, blood was collected from all animals at 24 hours post-treatment. qPCR of blood samples revealed no detectable vector in four out of five HSV-ABCA4 treated mice; one sample resulted in low-but-detectable vector genomes near the limit of quantitation of the assay (data not shown).

These in vitro and in vivo data indicate that HSV-ABCA4 capably transduced clinically relevant human retinal cells and induced deposition of full-length human ABCA4 in a dose-dependent manner without toxicity. Moreover, the vector could be effectively delivered to relevant cells in the retina via suprachoroidal administration, leading to full-length human ABCA4 expression without significant adverse effects. Taken together, these observations support the application of HSV-ABCA4 as a promising and innovative HSV-1-based therapy to fundamentally treat the molecular defect underlying Stargardt disease.

### Example 6: A Modified Herpes Simplex Virus Encoding Human Type VII Collagen for the Treatment of Recurrent Cicatrizing Conjunctivitis in a Patient with Dystrophic Epidermolysis Bullosa

Dystrophic epidermolysis bullosa (DEB) is a rare genetic blistering skin disease caused by mutations in the COL7A1 gene. It can be inherited in an autosomal dominant (DDEB) or recessive (RDEB) manner. A subset of individuals with DEB may develop ocular surface involvement, including abrasions, blistering and scarring, that can lead to impaired vision and eventual blindness. Current therapies are limited to removal of scar tissue and ophthalmic lubrication. The present study evaluated the topical use of a replication deficient herpes simplex virus type 1 vector engineered to deliver functional human type VII collagen (HSV-COL7).

A 13-year-old boy presented with DEB manifestations of ocular surface involvement and was treated on a compassionate use basis. The patient had severe ankyloblepharon reaching the central cornea in both eyes. The visual acuity was hand motion (log MAR, 3.00) in the right eye and 20/400 (log MAR, 1.30) in the left eye. Surgical symblepharon lysis with pannus removal was performed on the patient's right eye, and ophthalmic HSV-COL7 was administered three times per week for the first 2 weeks and then once weekly. After the epithelium was completely closed, HSV-COL7 applications were reduced to monthly administration.

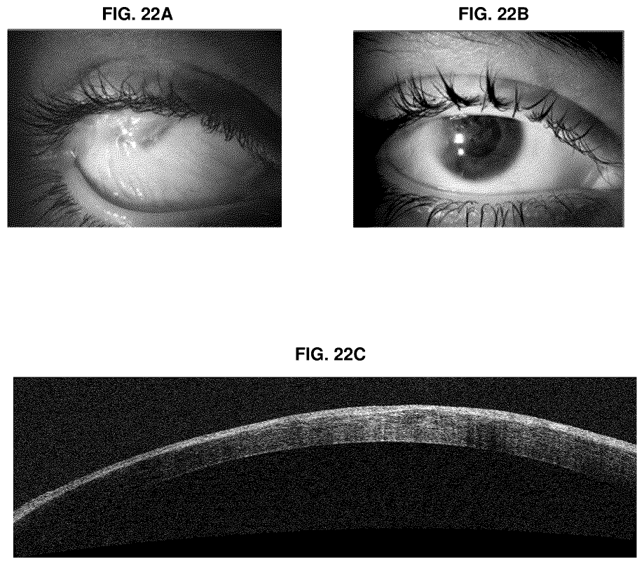

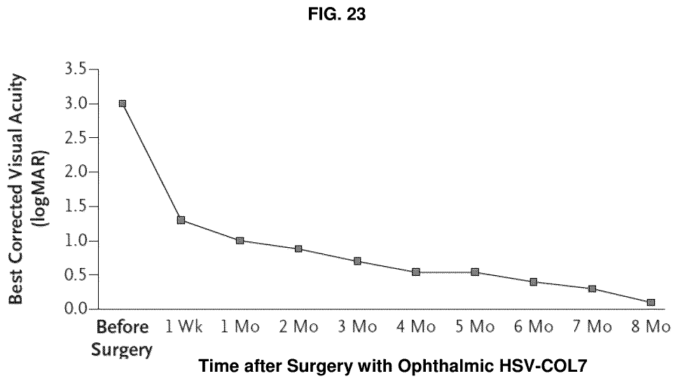

Full healing of the corneal epithelium was observed 3 months after the surgery and regular use of topical HSV-COL7 (total of 19 cumulative doses). Eight months after surgery, slit-lamp examination showed complete epithelial healing (FIGS. 22A-22B); images obtained by optical coherence tomography (OCT) showed no evidence of corneal scarring or infiltrates, and there was no evidence of corneal abnormalities or recurrence of symblepharon (FIG. 22C). Visual acuity in the patient's right eye improved from hand motion (log MAR, 3.00) before surgery to 20/25 (log MAR, 0.10) without correction at 8 months after the surgery (FIG. 23). Ocular application of HSV-COL7 was found to be well-tolerated in this patient with no signs of ocular HSV-1-like disease, establishing preliminary safety of the vector technology when repeatedly administered to the eye.

### Example 7: A Modified Herpes Simplex Virus Encoding Human Centrosomal Protein of 290 kDa for the Treatment of Leber Congenital Amaurosis (LCA)

The following example describes the use of a recombinant herpes simplex virus type 1 (HSV-1) that successfully encodes human Centrosomal protein of 290 kDa (see e.g. SEQ ID NOs: 574 and 738) and expressed full-length human CEP290 (see e.g. SEQ ID NO: 217) protein.

A recombinant HSV-1 is engineered to incorporate a human CEP290 (see SEQ ID NO: 738) expression cassette. The recombinant HSV-1 vector contains a heterologous promoter and polyA sequence (see Example 1). Viral plaques putatively containing the human CEP290 cassette are picked and screened by infection in a complementing cell line. High expressing clones, termed HSV-CEP290, are subsequently selected for additional in vitro and/or in vivo analysis, as well as its efficacy as a treatment of one or more conditions involving the eye as described herein. These results are not available at the time of filing.

1.-81. (canceled)

82.-101. (canceled)

102. A pharmaceutical composition comprising:
(a) a replication defective herpes simplex virus comprising a recombinant herpes simplex virus genome, wherein the recombinant herpes simplex virus genome comprises a polynucleotide encoding a beta-nerve growth factor (bNGF) polypeptide; and
(b) a pharmaceutically acceptable excipient.

103. The pharmaceutical composition of claim 102, wherein the recombinant herpes simplex virus genome is a recombinant herpes simplex virus type 1 (HSV-1) genome or a recombinant herpes simplex virus type 2 (HSV-2) genome.

104. The pharmaceutical composition of claim 102, wherein the recombinant herpes simplex virus genome comprises an inactivating mutation in a herpes simplex virus gene selected from the group consisting of Infected Cell Protein (ICP) 0, ICP4, ICP22, ICP27, ICP47, thymidine kinase (tk), Long Unique Region (UL) 41, and UL55.

105. The pharmaceutical composition of claim 102, wherein the recombinant herpes simplex virus genome comprises an inactivating mutation in one or both copies of the ICP4 gene.

106. The pharmaceutical composition of claim 102, wherein the bNGF comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 363, SEQ ID NO: 739, or SEQ ID NO: 740.

107. The pharmaceutical composition of claim 102, wherein the pharmaceutical composition is suitable for ocular, subretinal, intraocular, intravitreal, intracameral, topical, subcutaneous, subconjunctival, subtenon, subtenon capsule, intracameral, retrobulbar, systemic, parenteral, periocular, juxtascleral, anterior juxtascleral, posterior juxtascleral, oral, peribulbar, or suprachoroidal administration.

108. A method of providing prophylactic, palliative, or therapeutic relief of one or more signs or symptoms of an eye condition or disease in a subject in need thereof, the method comprising administering to the subject an effective amount of a pharmaceutical composition comprising:
(a) a replication defective herpes simplex virus comprising a recombinant herpes simplex virus genome, wherein the recombinant herpes simplex virus genome comprises a polynucleotide encoding a bNGF polypeptide; and
(b) a pharmaceutically acceptable excipient.

109. The method of claim 108, wherein the pharmaceutical composition is administered via injection, via injection to the eye, via subretinal injection, via intraocular injection, via intravitreal injection, via suprachoroidal injection, via intracameral injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally to the subject.

110. The method of claim 108, wherein the pharmaceutical composition is administered topically to the subject.

111. The method of claim 108, wherein the eye condition or disease is neurotrophic keratitis, diabetic retinopathy, dry eye disease, or retinitis pigmentosum.

112. The method of claim 108, wherein the eye condition or disease is neurotrophic keratitis.

113. The method of claim 108, wherein the recombinant herpes simplex virus genome is a recombinant HSV-1 genome or a recombinant HSV-2 genome.

114. The method of claim 108, wherein the recombinant herpes simplex virus genome comprises an inactivating mutation in a herpes simplex virus gene selected from the group consisting of ICP0, ICP4, ICP22, ICP27, ICP47, tk, UL41, and UL55.

115. The method of claim 108, wherein the recombinant herpes simplex virus genome comprises an inactivating mutation in one or both copies of the ICP4 gene.

116. The method of claim 108, wherein the bNGF comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 363, SEQ ID NO: 739, or SEQ ID NO: 740.

117. A pharmaceutical composition comprising:
(a) a replication defective herpes simplex virus comprising a recombinant herpes simplex virus genome, wherein the recombinant herpes simplex virus genome comprises a polynucleotide encoding a retinal-specific phospholipid-transporting ATPase polypeptide; and
(b) a pharmaceutically acceptable excipient.

118. The pharmaceutical composition of claim 117, wherein the recombinant herpes simplex virus genome is a recombinant HSV-1 genome or a recombinant HSV-2 genome.

119. The pharmaceutical composition of claim 117, wherein the recombinant herpes simplex virus genome comprises an inactivating mutation in a herpes simplex virus gene selected from the group consisting of ICP0, ICP4, ICP22, ICP27, ICP47, tk, UL41, and UL55.

120. The pharmaceutical composition of claim 117, wherein the recombinant herpes simplex virus genome comprises an inactivating mutation in one or both copies of the ICP4 gene.

121. The pharmaceutical composition of claim 117, wherein the retinal-specific phospholipid-transporting ATPase comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33.

122. The pharmaceutical composition of claim 117, wherein the pharmaceutical composition is suitable for ocular, subretinal, intraocular, intravitreal, intracameral, topical, subcutaneous, subconjunctival, subtenon, subtenon capsule, intracameral, retrobulbar, systemic, parenteral, periocular, juxtascleral, anterior juxtascleral, posterior juxtascleral, oral, peribulbar, or suprachoroidal administration.

123. A method of providing prophylactic, palliative, or therapeutic relief of one or more signs or symptoms of an eye condition or disease in a subject in need thereof, the method comprising administering to the subject an effective amount of a pharmaceutical composition comprising:
(a) a replication defective herpes simplex virus comprising a recombinant herpes simplex virus genome, wherein the recombinant herpes simplex virus genome comprises a polynucleotide encoding a retinal-specific phospholipid-transporting ATPase polypeptide; and
(b) a pharmaceutically acceptable excipient.

124. The method of claim 123, wherein the pharmaceutical composition is administered via injection, via injection to the eye, via subretinal injection, via intraocular injection, via intravitreal injection, via suprachoroidal injection, via intracameral injection, intraocularly, intravitreally, topically, subcutaneously, subconjunctivally, subtenonly, intracamerally, retrobulbarly, systemically, parenterally, periocularly, juxtasclerally, anterior juxtasclerally, posterior juxtasclerally, orally, peribulbarly, or suprachoroidally to the subject.

125. The method of claim 123, wherein the pharmaceutical composition is administered suprachoroidally to the subject.

126. The method of claim 123, wherein the eye condition or disease is Stargardt disease.

127. The method of claim 123, wherein the recombinant herpes simplex virus genome is a recombinant HSV-1 genome or a recombinant HSV-2 genome.

128. The method of claim 123, wherein the recombinant herpes simplex virus genome comprises an inactivating mutation in a herpes simplex virus gene selected from the group consisting of ICP0, ICP4, ICP22, ICP27, ICP47, tk, UL41, and UL55.

129. The method of claim 123, wherein the recombinant herpes simplex virus genome comprises an inactivating mutation in one or both copies of the ICP4 gene.

130. The method of claim 123, wherein the retinal-specific phospholipid-transporting ATPase comprises a sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 33.
